(19) United States
(12) Patent Application Publication
(10) Pub. No.: US 2014/0356414 A1
(43) Pub. Date: Dec. 4, 2014

(54) Targeted Crosslinked Multilamellar Liposomes

(71) Applicant: University of Southern California, Los Angeles, CA (US)

(72) Inventors: Pin WANG, Los Angeles, CA (US); Michael KK WONG, La Canada, CA (US); Andrew GRAY, Los Angeles, CA (US); Kye-Il JOO, Hermosa Beach, CA (US); Yarong LIU, Los Angeles, CA (US)

(73) Assignee: University of Southern California, Los Angeles, CA (US)

(21) Appl. No.: 14/295,014

(22) Filed: Jun. 3, 2014

Related U.S. Application Data
(60) Provisional application No. 61/830,636, filed on Jun. 3, 2013.
(60) Provisional application No. 61/830,608, filed on Jun. 3, 2013.

(51) Int. Cl. A61K 9/127; A61K 47/48; A61K 31/337; A61K 45/06; A61K 31/713; A61K 31/704

(52) U.S. Cl. CPC A61K 9/127; A61K 31/713; A61K 31/704; A61K 31/337; A61K 45/06; A61K 47/48815; A61K 47/48346
USPC 424/450; 514/44 A; 514/34

(57) ABSTRACT

A liposome composition that is useful for treating a subject in need of cancer treatment includes a cross-linked multilamellar liposome having an exterior surface and an interior surface. The interior surface defines a central liposomal cavity. The multilamellar liposome includes at least a first lipid bilayer and a second lipid bilayer. The first lipid bilayer is covalently bonded to the second lipid bilayer. At least one anticancer compound is disposed within the multilamellar liposome. Methods for treating subjects are also provided.

### CROSS-REFERENCE TO RELATED APPLICATIONS

This application claims the benefit of U.S. provisional application Ser. No. 61/830,636 filed Jun. 3, 2013 and U.S. provisional application Ser. No. 61/830,608 filed Jun. 3, 2013, the disclosures of which are hereby incorporated in their entirety by reference herein.

### STATEMENT REGARDING FEDERALLY SPONSORED RESEARCH OR DEVELOPMENT

The invention was made with Government support under Contract Nos. R01AI068978, R01CA170820, and P01CA132681 awarded by the Department of Health and Human Services. The Government has certain rights to the invention.

### SEQUENCE LISTING

The text file is Sequence_Listing.txt, created Jun. 3, 2014, and of size 4 KB, filed herewith, is hereby incorporated by reference.

### TECHNICAL FIELD

In at least one aspect, the present invention is related to liposome compositions for delivering therapeutic compounds such as anticancer compounds.

### BACKGROUND

For optimal anticancer treatment with cytotoxic drugs, it is necessary to sustain antitumor effects over a prolonged period at an efficacious drug concentration without inducing severe systemic toxicity. Therefore, as an alternative to conventional medicine for cancer therapeutics, nanoparticle-based drug delivery systems have been widely evaluated and utilized to modulate the toxicity profile of anticancer drugs and improve drug circulation time. Long-circulating liposomes, such as polyethylene glycol (PEG)-coated liposomes, have become one of the most popular nanocarriers for delivering therapeutics and have shown the ability to passively accumulate in tumors as a result of enhanced permeability and retention (EPR) effect. Ultimately, however, active targeting to tumor cells via the inclusion of a tumor-targeting molecule on the nanocarriers is expected to provide more effective cancer therapy. Some targeting molecules facilitate the binding of nanoparticles to the tumor endothelium, from where the nanoparticles can extravasate into the tumor environment. Once extravasated in the tumor environment, other targeting molecules will likely foster the active attachment of nanoparticles to tumor cells expressing the specific receptors for elevated antitumor activity.

Scientific investigations have identified diverse tumor-targeting molecules that can be exploited by nanoparticles to actively target cancer cell-specific markers with unique phenotypes in tumors. For example, it has been reported that drug carriers conjugated with targeting ligands, such as anti-Her2 antibody, folate, or transferrin (Tf) have achieved therapeutic benefit by successfully targeting human epidermal receptors (HER), folate receptors and transferrin receptor (TfR), respectively, all of which are overexpressed on tumor cells. The cell- or tissue-specific ligand-receptor interaction contributes to the increased efficacy as a result of enhanced uptake of the complex into tumor cells by receptor-mediated endocytosis. However, a major obstacle against the clinical application of this targeting strategy has been the poor penetration of the targeted payload through the vascular wall and into the tumor parenchyma, especially in solid tumors, which have a high interstitial pressure.

Accordingly, there is a need for improved liposome compositions

### SUMMARY

The present invention solves one or more problems of the prior art by providing in at least one embodiment, a liposome composition that is useful for treating a subject in need of cancer treatment. The composition includes a cross-linked multilamellar liposome having an exterior surface and an interior surface. The interior surface defines a central liposomal cavity. The multilamellar liposome includes at least a first lipid bilayer and a second lipid bilayer. The first lipid bilayer is covalently bonded to the second lipid bilayer. At least one anticancer compound is disposed within the multilamellar liposome.

In another embodiment, a liposome composition that is useful for treating a subject in need of cancer treatment is provided. The composition includes a crosslinked multilamellar liposome having an exterior surface and an interior surface. The interior surface defines a central liposomal cavity. The multilamellar liposome includes at least a first lipid bilayer and a second lipid bilayer. The first lipid bilayer is characteristically covalently bonded to the second lipid bilayer. A targeting peptide is covalently bonded to the exterior surface of the multilamellar liposome. At least one anticancer compounds disposed is disposed within the multilamellar liposome.

In another embodiment, a method for treating a subject in need of cancer treatment is provided. The method includes a step of identifying a subject having cancer. A therapeutically effective amount of a composition is administered to a subject. The composition includes a crosslinked multilamellar liposome having an exterior surface and an interior surface. The interior surface defines a central liposomal cavity. The multilamellar liposome includes at least a first lipid bilayer and a second lipid bilayer. The first lipid bilayer is covalently bonded to the second lipid bilayer. At least one anticancer compound is disposed within the liposome.

In still another embodiment, a method for treating subject in need of cancer treatment is provided. The method includes a step of identifying a subject having drug resistant cancer, and in particular multidrug resistant cancer. A therapeutically effective amount of a composition is administered to a subject. The composition includes a crosslinked multilamellar liposome having an exterior surface and an interior surface. The interior surface defines a central liposomal cavity. The multilamellar liposome includes at least a first lipid bilayer and a second lipid bilayer. The first lipid bilayer is covalently bonded to the second lipid bilayer. At least one anticancer compound is disposed within the liposome.

### BRIEF DESCRIPTION OF THE DRAWINGS

FIG. 1A. Schematic illustration of a crosslinked multilamellar liposome;

FIG. 1B. Schematic flowchart illustrating the preparation of crosslinked multilamellar liposomes of FIG. 1A;

FIG. 2A. Schematic illustration of a crosslinked multilamellar liposome having targeting peptides;

FIG. 2B. Schematic flowchart illustrating the preparation of crosslinked multilamellar liposomes of FIG. 2A;

FIG. 3. Schematic flowchart illustrating targeting of multiple sites of molecules involved in castration resistant prostate cancer;

FIG. 4. Schematic flowchart showing targeting multiple pathways involved in castration resistant prostate cancer;

FIG. 5. Schematic showing examples of androgen receptor siRNA targets;

FIGS. 6A-G. (A-C) The hydrodynamic size distribution of PEGylated ULs (A), DLLs (B), and CMLs (C), as measured by dynamic light scattering. (D) The mean diameter and polydispersity of ULs, DLLs and CMLs. (E, F) Visualization of unilamellar and multilamellar structure of vesicles. Cryo-electron microscopy images of ULs (E) and CMLs (F). (G) Cryo-electron microscopy images of CML showing the stacked lipid bilayers. The boxed region is enlarged in the right panel.

FIGS. 7A-E. Enhanced vesicle stability and sustainable release kinetics of CMLs. (A) Encapsulation efficiency of doxorubicin (Dox) into the UL, DLL, or CML. (B) Total Dox loading per phospholipids (μg/mg). (C) In vitro release kinetics of doxorubicin from ULs, DLLS, and CMLs. (D) In vitro cytotoxicity of free Dox, UL-Dox, DLL-Dox, and CML-Dox in B16 melanoma tumor. The cytotoxicity was measured by a standard XTT assay. Error bars represent the standard deviation of the mean from triplicate experiments. (E) In vitro cytotoxicity of free Dox, UL-Dox, and CML-Dox in HeLa cells. The cytotoxicity was measured by a standard XTT assay. Error bars represent the standard deviation of the mean from triplicate experiments.

FIGS. 8A-B. Caveolin-mediated internalization of CMLs and subsequent intracellular processing. HeLa cells were incubated with DiD-labeled CML particles for 30 min at 4° C. to synchronize internalization. The cells were then shifted to 37° C. for 15 min, fixed, permeabilized, and immunostained with anti-caveolin-1 or anti-clathrin antibody. The boxed regions are enlarged in the bottom panels. (A) Quantification of CML particles colocalized with caveolin-1 or clathrin signals after 15 min of incubation. Overlap coefficients were calculated with Manders' overlap coefficients (MOC) by viewing more than 50 cells of each sample using the Nikon NIS-Elements software. Error bars represent the standard deviation of the mean from analysis of multiple images. (B) Inhibition of energy-dependent internalization by incubation at 4° C., clathrin-dependent internalization by chlorpromazine (CPZ, 25 μg/ml), and caveolin-dependent internalization by nystatin (50 μg/ml) and methyl-β-cyclodextrin (MβCD, 15 mM). The uptake of DiD-labeled CML particles was determined by measuring DiD fluorescence.

FIGS. 9A-B. In vivo toxicity and tolerability. C57/BL6 mice were administered a single intravenous injection with CML-Dox or free Dox. (A) Average mouse weight loss over time. Data are presented as the percentage of the initial weights for mice treated with no injection (black line), CML-Dox (20 mg/kg Dox equivalent, red line; 40 mg/kg Dox equivalent, blue line), or free Dox (20 mg/kg Dox equivalent, green line; 40 mg/kg Dox equivalent, orange line). Error bars represent the standard deviation of the mean; n=2 for each treatment group (*p<0.05, ***p<0.005). (B) Histologic appearance of cardiac tissues obtained from C57/BL6 mice with no drug treatment or administered a single intravenous injection with free Dox or CML-Dox at 20 mg/kg Dox equivalent.

FIGS. 10A-C. Antitumor effect of Dox-loaded CMLs, ULs, and DLLs in the B16 melanoma tumor model. (A) Tumor growth was measured after treatment with no injection (black line), CML-Dox (1 mg/kg DOX equivalent, blue dotted line; 4 mg/kg DOX equivalent, blue solid line), UL-Dox (1 mg/kg DOX equivalent, green dotted line; 4 mg/kg DOX equivalent, green solid line), or DLL-Dox (1 mg/kg DOX equivalent, red dotted line; 4 mg/kg DOX equivalent, red solid line). Error bars represent the standard error of the mean; n=6 for each treatment group (*p<0.05, **p<0.01). (B) Excised tumors from each treatment group at 16 days after tumor inoculation. (C) Average mouse weight loss over the duration of the experiment.

FIGS. 11A-D Biodistribution of drug carriers and accumulation of Dox in tumors. (A) Preparation of ⁶⁴Cu-AmBaSar-labeled liposomes. (B) Biodistribution of liposomes in different tissues at 24 h after injection with ⁶⁴Cu-AmBaSar-labeled UL, DLL, or CML shown as percentage of injection dose per g of tissues (% ID/g). (C) The pharmacokinetics of Dox in plasma of C57/BL6 mice bearing B16 tumors injected with free Dox, UL-Dox, DLL-Dox, or CML-Dox at a dose of 10 mg/kg Dox equivalent. At given time intervals, blood was collected by retro-orbital bleeding. (D) Accumulation of Dox in tumors. C57/BL6 mice bearing B16 tumors were intravenously injected with free Dox, UL-Dox, DLL-Dox, or CML-Dox at a dose of 10 mg/kg Dox equivalent. The mean Dox concentrations in tumors were shown. Error bars represent the standard deviation of the mean; n=3 for each treatment group.

FIG. 12A-B. (A) The hydrodynamic size distribution of iRGD-cMLVs measured by dynamic light scattering (DLS). Data represented the mean±SD of at least three experiments with n=3. (B) In vitro release kinetics of doxorubicin (Dox) from iRGD-cMLVs. Error bars represent standard error of the mean; n=3 for each formulation.

FIG. 13A-D. In vitro cytotoxicity, binding and internalization of iRGD-cMLVs and cMLVs in tumor cells. (A, B) In vitro cytotoxicity of cMLV(Dox) and iRGD-cMLV(Dox) in 4T1 tumor (A) and multidrug-resistant JC cells (B). The cytotoxicity was measured by a standard XTT assay. Error bars represent the standard deviation of the mean from triplicate experiments. (C, D) Binding and internalization of cMLV(Dox) and iRGD-cMLV(Dox) to 4T1 cells. 4T1 cells were incubated with cMLV(Dox) and iRGD-cMLV(Dox) for 30 min at 4° C. (C) or 2 h at 37° C. (D). Both binding and cellular uptake of nanoparticles were determined by measuring doxorubicin fluorescence using flow cytometry. Statistical analysis was performed with Student's t-test. Error bars represent the standard deviation of the mean from triplicate experiments.

FIG. 14A-C. (A, B) Quantification of cMLV and iRGD-cMLV particles colocalized with clathrin (A) or caveolin-1 signals (B) after 15 min of incubation. Overlap coefficients were calculated using Manders' overlap coefficients by viewing more than 30 cells of each sample using the Nikon NIS-Elements software. Error bars represent the standard deviation of the mean from analysis of multiple images (***: P<0.005). (C) Inhibition of clathrin-dependent endocytosis by chlorpromazine (CPZ, 25 μg/ml) and caveolin-dependent internalization by Filipin (10 μg/ml). The uptake of DiD-labeled cMLV and DiD-labeled iRGD-cMLV nanoparticles was determined by measuring DiD fluorescence via flow cytometry. Error bars represent the standard deviation of the mean from triplicate experiments (*P<0.05, **P<0.01).

FIG. 15A-D. Antitumor effect of iRGD-cMLVs and cMLVs in the 4T1 breast tumor model. (A) Schematic diagram of the experimental protocol for the in vivo tumor study. (B) Tumor growth was measured after treatment without injection (control), cMLV(Dox) and iRGD-cMLV(Dox) (2 mg/kg Dox equivalents). Error bars represent standard error of the mean; n=5 for each treatment group (*P<0.05). (C) Average mouse weight loss over the duration of the experiment. (D) Tumor weight of excised tumors from each treatment group at 25 days after tumor inoculation. Error bars represent standard error of the mean; n=5 for each treatment group.

FIG. 16A-H. Characteristics of cMLV(Dox+PTX). (A-C). The hydrodynamic size distribution of cMLV(Dox), cMLV(PTX), and cMLV(Dox+PTX) measured by dynamic light scattering. The mean hydrodynamic diameter (HD) and polydispersity index (PI) of cMLV(Dox), cMLV(PTX), and cMLV(Dox+PTX) are indicated on the graph. (D, E) Effects of co-encapsulation of Dox and PTX on loading capability and drug release kinetics profiles of cMLVs. The encapsulation efficiency (D) and loading efficacy (E) of drugs in cMLV(combined drugs) and cMLV (single drug). (F-H) In vitro release kinetics of doxorubicin and paclitaxel from dual-drug loaded cMLVs and single-drug loaded cMLVs. Error bars represent the standard deviation of the mean from triplicate experiments.

FIG. 17A-H. Determination of the ratio of drug combinations to induce synergy. (A, B) In vitro cytotoxicity of three weight ratios (5:1, 3:3 and 1:5) of Dox and PTX in cMLV formulations (A) or solution (B) in B16 melanoma tumor or 4T1 breast tumor cell lines. The cytotoxicity was measured by a standard XTT assay. (C) Combination Index (CI) histogram for cMLV (different drug combinations) exposed to cultured B16 and 4T1 tumor cells. (D) Combination Index histogram for different ratios of drug combination in solution exposed to culture B16 and 4T1 tumor cells. The surviving cell fraction from three replicates was averaged and analyzed by nonlinear regression. The histogram presents the CI values obtained at a fraction of 0.5. Error bars represent the standard deviation of the mean from triplicate experiments. (E) Immunoblot analysis of phosphorylated ERK in B16 cells treated by cMLV(Dox+PTX) with three dose ratios: 5:1, 3:3 and 1:5. β-actin was used as a control. (F) Quantification of phosphorylated ERK shown in (E). Protein amounts were estimated by densitometry of immunoblots. Error bars represent SD. (G, H) The IC₅₀ values of individual drugs either in cMLVs or in solution for B16 melanoma cells and 4T1 breast cancer cells are shown in FIGS. 17 G and H, respectively.

FIG. 18A-C. Drug ratio-dependent efficacy of cMLV(Dox+PTX) in tumor treatment. (A) Tumor growth was measured after treatment with PBS, 3.33 mg/kg Dox+0.67 mg/kg PTX, 2 mg/kg Dox+2 mg/kg PTX, 0.67 mg/kg Dox+3.33 mg/kg PTX, either in cMLVs or in solution every three days. Tumor growth and body weights were monitored until the end of the experiment. Error bars represent standard error of the mean, n=6 for each treatment group (*p<0.05, **p<0.01). (B) Average mouse weight loss over the duration of the experiment. (C) Survival curves for 4T1 bearing mice treated with PBS, 3.33 mg/kg Dox+0.67 mg/kg PTX, 2 mg/kg Dox+2 mg/kg PTX, 0.67 mg/kg Dox+3.33 mg/kg PTX either in cMLVs or in solution every three days. The survival rates are presented as Kaplan-Meier curves. The survival curves of individual groups were compared by a log-rank test.

FIG. 19. Drug ratio-dependent efficacy of co-encapsulated Dox:PTX on tumor cell apoptosis. 4T1 Tumor bearing mice were treated with PBS, 8.333 mg/kg Dox+1.667 mg/kg PTX, 5 mg/kg Dox+5 mg/kg PTX, 1.667 mg/kg Dox+8.33 mg/kg PTX, either in cMLVs or in solution. Three days after injection, tumors were excised. Apoptotic cells were detected by TUNEL assay (green), and co-stained by nuclear staining DAPI (blue). Scale bar represents 50 μm. (A) Quantification of apoptotic positive cells in 4T1 tumor. To quantify TUNEL positive cells, 4 regions of interest (ROI) were randomly chosen per image at ×2 magnification. Within one region, area of TUNEL positive nuclei, and area of nuclear staining were counted by software. The data is expressed as % total nuclear area stained by TUNEL in the region. Data represented as mean±SD (n=3).

FIG. 20. In vivo toxicity. Histologic appearance of cardiac tissues obtained from C57/BL6 mice with no drug treatment or administered a single intravenous injection with three dose ratios of Dox and PTX (5:1, 3:3 and 1:5) in solutions or cMLV formulations at 10 mg/kg total drug equivalent. Scale bar represents 100 μm.

FIG. 21A-C. (A) In vivo maintenance of Dox:PTX ratios in cMLV formulations. (B,C) Tumor bearing mice were treated with PBS, 8.333 mg/kg Dox+1.667 mg/kg PTX, 5 mg/kg Dox+5 mg/kg PTX, 1.667 mg/kg Dox+8.33 mg/kg PTX, either in cMLVs (B) or in solution (C). 24 h after injection, tumors were excised and drug concentrations of Dox and PTX were measured by HPLC. All data are shown as the means of triplicate experiments.

FIG. 22A-E. Overcoming drug resistance by codelivery of Dox and PTX via cMLVs (D: Dox; T: PTX). (A, B) In vitro cytotoxicity of cMLV(single drug) and cMLV(drug combinations) in B16 melanoma tumor cells (A) and 4T1 breast tumor cells (B). (C, D, E) In vitro cytotoxicity of cMLV(single drug) and cMLV(drug combinations) in drug-resistant JC cells (C), B16-R cells (D) and 4T1-R cells (E). IIP_{Cmax} was determined by incorporating three parameters (IC₅₀, D and m) in the median effect model into the following equation: IIP_{Cmax}=log (1+(Cmax/IC₅₀)^{m}). Data are represented as mean±SD (n=3). Asterisks indicate statistical significance between two groups (*P<0.05, **P<0.01).

FIG. 23A-D. Cellular uptake of Dox and PTX (D: Dox; T: PTX). (A, B) Total cellular uptake of Dox (A) and PTX (B) into 4T1 cells. 4T1 cells were exposed to cMLV(D), cMLV(T), cMLV(D+T), and D+T in solution. The final concentrations of Dox and PTX were 1 μg/ml for each group. (C, D) Total cellular uptake of Dox (C) and PTX (D) in JC cells. JC cells were exposed to cMLV(D), cMLV(T), cMLV(D+T), and D+T. The final concentrations of Dox and PTX were 5 μg/ml for each group. The uptake of Dox and PTX was normalized to protein content measured with the BCA assay. All data are shown as the means of triplicate experiments from three different nanoparticle preparations. Asterisks indicate statistical significance between two groups (*P<0.05, **P<0.01).

FIG. 24A-B. Effect of codelivered nanoparticles on P-gp expression (D: Dox; T: PTX). (A) 4T1 cells were exposed to cMLV(D), cMLV(T), cMLV(D+T), and D+T with the same concentration of Dox and PTX (1 μg/ml). (B) JC cells were exposed to cMLV(D), cMLV(T), cMLV(D+T), and D+T with the same concentration of Dox and PTX (5 μg/ml). P-gp expression was detected by P-gp-specific antibody via flow cytometry. Data are represented as mean±SD (n=3). Asterisks indicate statistical significance between two groups (*P<0.05, **P<0.01).

FIG. 25A-D. In vivo efficacy of drug combinations via cMLVs in a 4T1 tumor model. (A) Schematic diagram of the experimental protocol for in vivo 4T1 tumor study in BALB/c mice. (B) Tumor growth was measured after treatment with PBScMLV (2 mg/kg Dox), cMLV (2 mg/kg PTX) or cMLV (2 mg/kg Dox+2 mg/kg PTX) Error bars represent standard error of the mean, n=6 for each treatment group (**p<0.01). (C) Average mouse weight loss over the duration of the experiment. (D) Survival curves for 4T1-bearing mice treated with PBS, cMLV 2 mg/kg Dox), cMLV (2 mg/kg PTX), or cMLV (2 mg/kg Dox+2 mg/kg PTX). Survival end point was set when the tumor volume reached 1000 mm³. The survival rates were presented as Kaplan-Meier curves. The survival curves of individual groups were compared by a log-rank test.

FIG. 26A-B. Effect of codelivered cMLVs on tumor apoptosis (D: Dox; T: PTX). (A, B) Mice bearing either 4T1 tumor or multidrug-resistant JC tumor were injected intravenously through the tail vein with cMLV (5 mg/kg Dox), cMLV (5 mg/kg PTX), 5 mg/kg Dox+5 mg/kg PTX, or cMLV (5 mg/kg Dox+5 mg/kg PTX). Three days after injection, tumors were excised. Apoptotic cells were detected by a TUNEL assay, followed by nuclear costaining with DAPI. (A, B) Quantification of apoptotic cells in 4T1 (A) and JC (B) tumors. To quantify TUNEL-positive cells, 4 regions of interest (ROI) were randomly chosen per image at ×2 magnification. Within one region, area of TUNEL-positive nuclei and area of nuclear staining were counted. The data are expressed as % total nuclear area stained by TUNEL in the region. Data are represented as mean±SD (n=3).

FIG. 27A-B. Effect of codelivered cMLVs on P-gp expression in tumors. Mice bearing 4T1 tumor and multidrug-resistant tumor JC were injected intravenously through the tail vein with cMLV (5 mg/kg Dox), cMLV (5 mg/kg PTX), 5 mg/kg Dox+5 mg/kg PTX, or cMLV (5 mg/kg Dox+5 mg/kg PTX). Three days after injection, tumors were excised, and stained by P-gp-specific antibody, followed by nuclear costaining with DAPI. (A, B) Quantification of P-gp-positive cells in 4T1 (A) and JC (B) tumors. To quantify P-gp-positive cells, 4 regions of interest (ROI) were randomly chosen per image at ×2 magnification. Within one region, area of P-gp-positive nuclei and area of nuclear staining were counted. The data are expressed as % total nuclear area that is P-gp-positive in the region. Data are represented as mean±SD (n=3).

FIG. 28. Histologic appearance (hematoxylin and eosin staining) of heart tissues by light microscopy isolated on day 3 after a single intravenous injection of PBS (left), 5 mg/kg Dox+5 mg/kg PTX in solution (middle) and cMLV(5 mg/kg Dox+5 mg/kg PTX) (right).

FIG. 29. RRL-CML[siRNA] nanoparticles encapsulating siRNAs targeting the androgen receptor can knock down its expression in human prostate cancer cells. LNCaP cells were cultured for 48 hours in standard media containing RRL-CML[siRNA] nanoparticles that encapsulated either a universal negative control siRNA (RRL-CML[NC1]), one of four different siRNAs directed against the AR (RRL-CML[AR siRNA 1-4]), or a pool of all four of the siRNAs directed against the AR (RRL-CML[AR siRNA Pool]. After treatment, cells were harvested and total RNA isolated. This was used as a template to generate cDNA using random hexamers as primers. Relative AR expression in each of the treatment groups was assessed using real-time QPCR. The AR expression of untreated LNCaP cells was arbitrarily assigned as having a value of 1. Bars represent mean relative AR expression+/−SEM. Relative AR expression in each treatment group was compared to that of untreated LNCaP cells by Student's t test. ** indicates p<0.01. *** indicates p<0.00. “RRL-CML[NC1]” refers to RRL-CML nanoparticles containing a proprietary scrambled negative control siRNA pair from Integrated DNA Technology. “RRL-CML[AR siRNA 1]” refers to RRL-CML nanoparticles containing SEQ ID numbers 3 and 4 (heterodimerized to each other before encapsulation); “RRL-CML[AR siRNA 2]” refers to RRL-CML nanoparticles containing SEQ ID numbers 5 and 6 (heterodimerized to each other before encapsulation); “RRL-CML[AR siRNA 3]” refers to RRL-CML nanoparticles containing SEQ ID numbers 7 and 8 (heterodimerized to each other before encapsulation); “RRL-CML[AR siRNA 4]” refers to RRL-CML nanoparticles containing SEQ ID numbers 9 and 10 (heterodimerized to each other before encapsulation)

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

### ABBREVIATIONS

“° C.” means degrees Celcius.

“μmol” means micromoles.

“ADT” means androgen deprivation therapy.

“ANOVA” means analysis of variance.

“AR” means androgen receptor.

“ATCC” means American type culture collection.

“CI” means combination index.

“CML” means crosslinked multilamellar liposome.

“CML(D)” means CML containing doxorubicin.

“CML(D+T)” means CML containing doxorubicin plus taxol.

“CML(Dox)” means CML containing doxorubicin.

“CML-Dox” means CML containing doxorubicin.

“CML(PTX)” means CML containing taxol.

“CML(T)” means CML containing taxol.

“cMLV” means crosslinked multilamellar vesicle which in the context of the present invention is the same as a crosslinked multilamellar liposome.

“cMLV(D)” means cMLV containing doxorubicin.

“cMLV(Dox)” means cMLV containing doxorubicin.

“cMLV(D+T)” means cMLV containing doxorubicin plus taxol.

“cMLV(Dox+PTX)” means cMLV containing doxorubicin plus taxol.

“cMLV(PTX)” means cMLV containing taxol.

“cMLV(T)” means cMLV containing taxol.

“CPZ” means chlorpromazine.

“CRPC” means castration resistant prostate cancer.

“CTxB” means cholera toxin binding subunit.

“DAPI” means 4′,6-diamidino-2-phenylindole.

“DBD” means DNA binding domain.

“DLL” means doxil-like liposome.

“DLL-Dox” means DLL containing doxorubicin.

“DLS” means dynamic light scattering.

“DMEM” means Dulbecco's modified Eagle's medium.

“DNA” means deoxyribonucleic acid.

“DOPC” means 1,2-dioleoyl-sn-glycero-3-phosphocholine.

“DOPG” means 1,2-dioleoyl-sn-glycero-3-phospho-(1′-rac-glycerol).

“Dox” means doxorubicin.

“DTT” means dithiothreitol.

“EDC” means 1-Ethyl-3-[3-dimethylaminopropyl]carbodiimide hydrochloride.

“EEA1” means early endosome antigen 1.

“EPR” means enhanced permeability and retention effect

“ER” means endoplasmic reticulum.

“ERK” means extracellular-signal-regulated kinase.

“fa” means fraction of affected cells.

“FBS” means fetal bovine serum.

“g” means gravities.

“g/min” means grams per minute.

“h” means hours.

“H” means hinge region.

“HBS” means HEPES buffered saline.

“HD” means hydrodynamic diameter.

“HER” means human epidermal receptor.

“HPLC” means high-performance liquid chromatography.

“HPV31” means human papillomavirus type 31.

“IgG” means immunoglobulin gamma.

“iRGD-cMLV” means cMLV conjugated to iRGD.

“kDa” means kiloDaltons.

“LBD” means ligand binding domain.

“MβCD” means methyl-β-cyclodextrin.

“MAPK” means mitogen-activated protein kinases.

“MDR” means multidrug resistance.

“MFI” means mean fluorescence intensity.

“MgCl₂” means magnesium chloride.

“ml/min” means milliliters per minute.

“mM” means millimolar.

“MPB-PE” means 1,2-dioleoyl-sn-glycero-3-phosphoethanolamine-N-[4-(p-maleimidophenyl)butyramide.

“nm” means nanometers.

“NRP-1” means neuropilin-1.

“PBS” means phosphate buffered saline.

“PEG” means polyethylene glycol.

“PET” means positron emission tomography.

“P-gp” is P-glycoprotein.

“PI” means polydispersity index.

“PTX” means paclitaxel.

“QPCR” means quantitative polymerase chain reaction.

“RNA” means ribonucleic acid.

“ROI” means region of interest.

“RPM” means revolutions per minute.

“RRL-CML” means CML conjugated to RRL.

“RRL-CML[siRNA]” means RRL-CML containing siRNA(s).

“s” means seconds.

“SNHS” means N-hydroxysulfosuccinimide.

“siRNA” means small interfering RNA.

“SV40” means simian virus 40.

“TAC” means time activity curves.

“TAD” means transactivating domain.

“TDEC” means tumor derived endothelial cell.

“Tf” means transferrin.

“TfR” means transferrin receptor.

“TGN38” means trans-Golgi network protein 2.

“TUNEL” means terminal deoxynucleotidyl transferase dUTP nick end labeling.

“UL” means unilamellar liposome.

“UL-Dox” means UL containing doxorubicin.

“PEG” is poly(ethylene glycol).

The term “subject” refers to a human or animal, including all mammals such as primates (particularly higher primates), sheep, dog, rodents (e.g., mouse or rat), guinea pig, goat, pig, cat, rabbit, and cow.

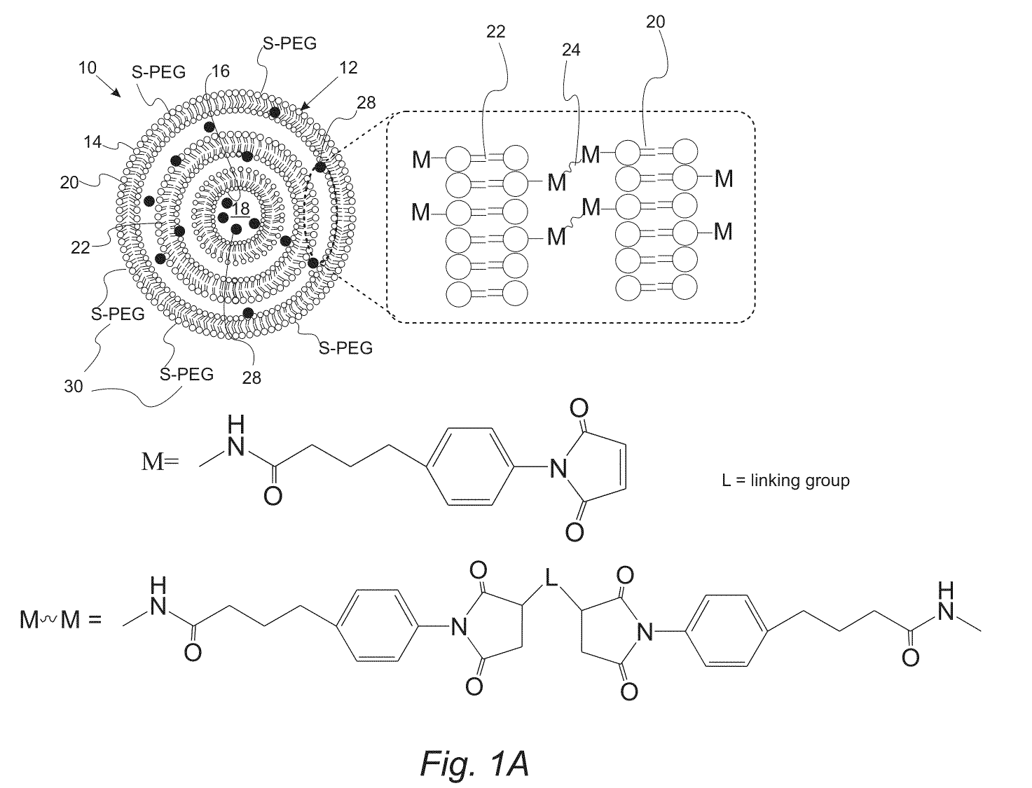

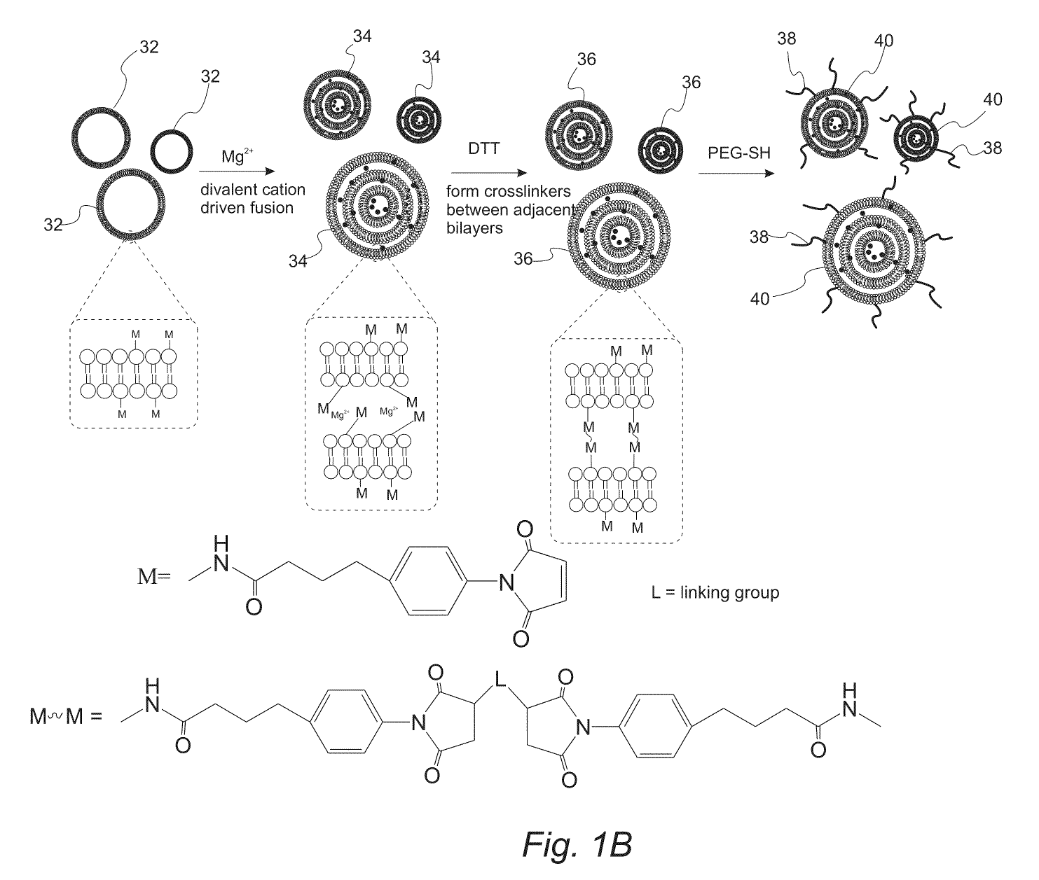

With reference to FIG. 1A, a liposome composition that is useful for treating a subject in need of cancer treatment is illustrated. Composition 10 includes a cross-linked multilamellar liposome 12 having an exterior surface 14 and an interior surface 16. Interior surface 16 defines a central liposomal cavity 18. Multilamellar liposome 12 includes at least a first lipid bilayer 20 and a second lipid bilayer 22. First lipid bilayer 20 is covalently bonded to second lipid bilayer 22 by covalent bond 24. In one refinement, the lipid bilayers are covalently bonded by ether bonds and/or thioether bonds. Typically, multilamellar liposome 12 includes at least one additional lipid bilayer such as third lipid bilayer 26 which is covalently bonded to second lipid bilayer 22. In a refinement, multilamellar liposome 12 includes on average from 2 to 10 lipid bilayers. In another refinement, multilamellar liposome 12 includes on average from 3 to 9 lipid bilayers. In still another refinement, multilamellar liposome 12 includes on average from 3 to 6 lipid bilayers. At least one anticancer compound 28 is disposed within multilamellar liposome 12. In one refinement, cancer compound 28 is disposed within liposomal cavity 18. In another refinement, cancer compound 28 is disposed within the lipid bilayers, for example lipid bilayers 20, 22 and any additional lipid layers. In still other refinements, cancer compound 28 is disposed within liposomal cavity 18 and the lipid bilayers. In some variations, poly(ethylene glycol) groups 30 are covalently bonded to the exterior surface of the liposome in order to improve water solubility. In a refinement, the poly(ethylene glycol) groups have a weight average molecular weight from about 400 to 2500 Daltons. In another refinement the poly(ethylene glycol) groups include from 9 to 45 repeat units of —OCH₂CH₂—.

With reference to FIG. 1B, a schematic flowchart illustrating the preparation of the multilamellar liposomes of FIG. 1A is provided. This method is adapted from Moon et al., (2011) Interbilayer-crosslinked Multilamellar Vesicles as Synthetic Vaccines for Potent Humoral and Cellular Immune Responses; Nat. Mater. 10, 243-251; the entire disclosure of this publication is hereby incorporated by reference. The preparation is based on the conventional dehydration-rehydration method. In general, the liposomes are formed through covalently crosslinking functionalized headgroups of adjacent lipid bilayers and in particular the incorporation of a thiol-reactive maleimide headgroup lipid (e.g., N-(3-Maleimide-1-oxopropyl)-L-α-phosphatidylethanolamine, MPB-PE) onto the surface of unilamellar liposome 32. In step a), divalent cation-triggered vesicle fusion yields a multilamellar structure 34. In step b), interbilayer crosslinking across the opposing sides of lipid bilayers through the reactive headgroups with dithiothreitol (DTT) generates crosslinked multilamellar liposomes 36. In step c, PEG groups 38 are added to the surface of the crosslinked multilamellar liposome 36 with thiol-terminated PEG to form pegylated crosslinked multilamellar liposomes 40, which further improves vesicle stability and blood circulation half-life.

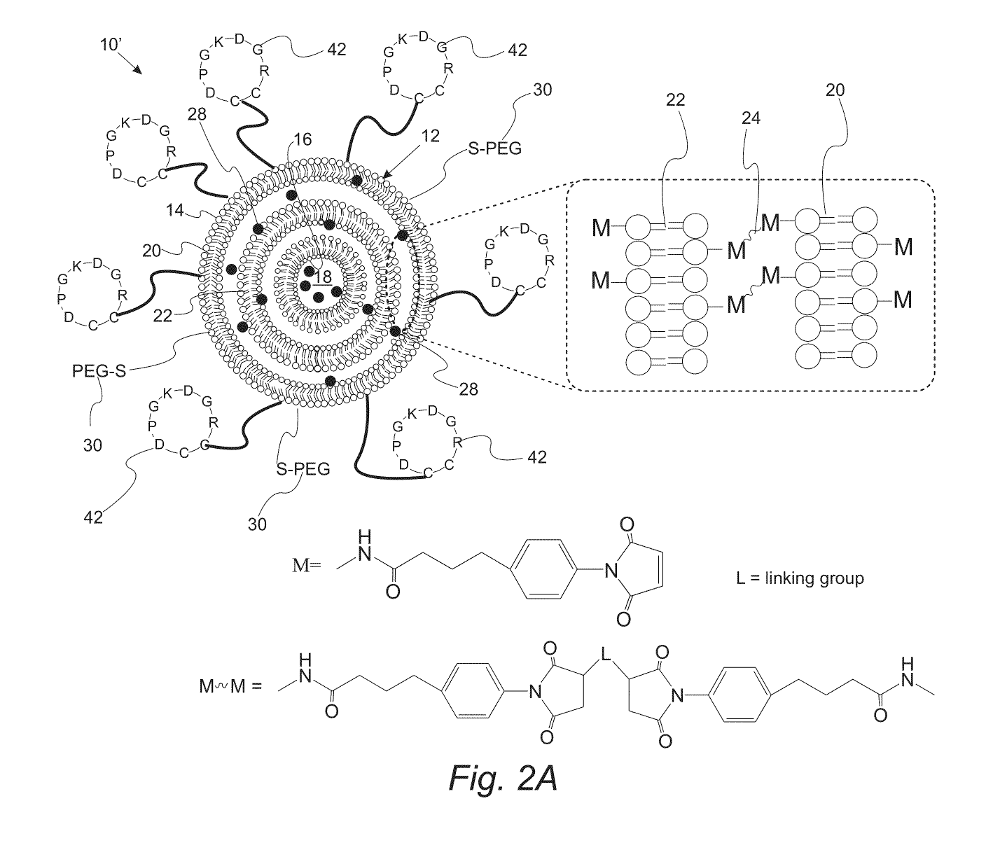

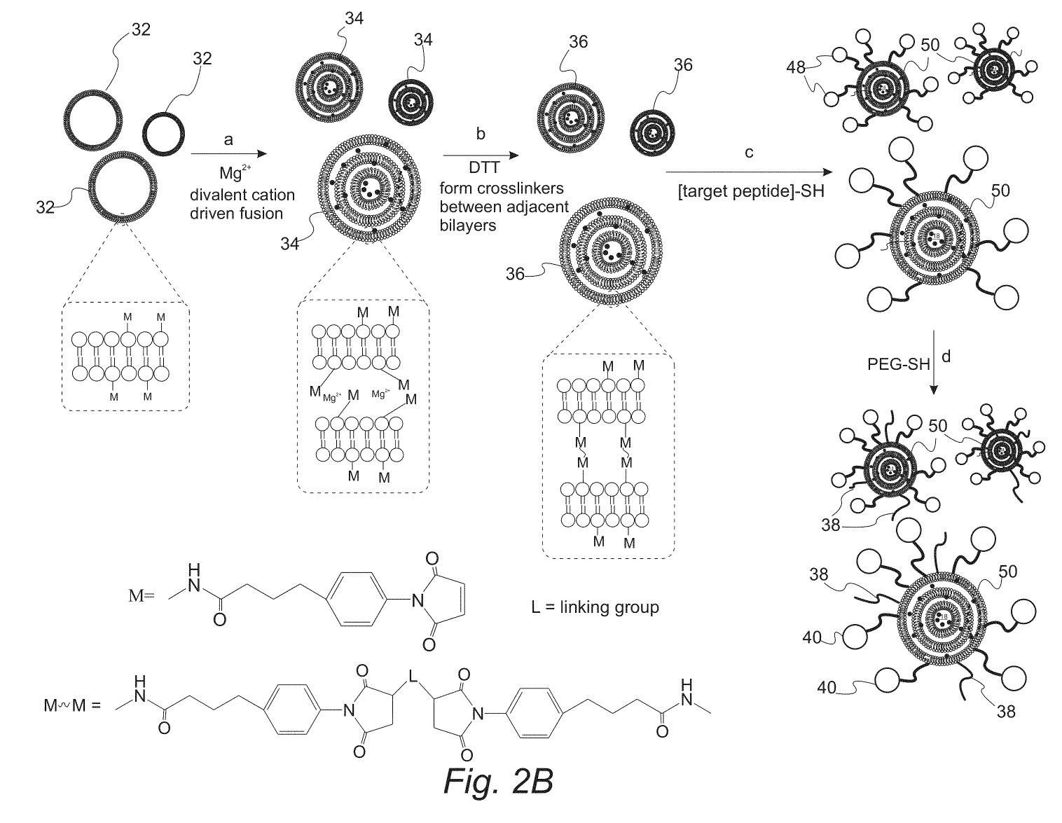

With reference to FIG. 2A, a liposome composition having a targeting peptide is schematically illustrated. The present embodiment provides a nanoparticle drug delivery system having a targeting peptide linked to a cross-linked multilamellar liposome (CML). The targeting peptide is a means for actively targeting the CML to a specific tissue, organ or site in need of therapy, for delivery of the CML-loaded drug(s). For example, the targeting peptide is a means for actively targeting the CML to a specific tumor or to specific tumors for delivery of anticancer drug(s) loaded in the CML. That is, the targeting peptide has specific affinity for a ligand on at least one type of tumor cell and/or tumor-associated tissue, thereby actively targeting the CML to the corresponding tumor(s) for drug delivery. In this way, a targeting peptide-linked CML increases delivery of the loaded anticancer drug(s) to the tumor, tumor cells, or tumor tissues compared to the passive delivery of a loaded CML without a targeting peptide. In particular, liposome composition 10′ includes a cross-linked multilamellar liposome 12 having an exterior surface 14 and an interior surface 16. Interior surface 16 defines a central liposomal cavity 18. Multilamellar liposome 12 includes at least a first lipid bilayer 20 and a second lipid bilayer 22. First lipid bilayer 20 is covalently bonded to second lipid bilayer 22 by covalent bond 24. In one refinement, the lipid bilayers are covalently bonded by ether bonds and thioether bonds. Typically, multilamellar liposome 12 includes at least one additional lipid bilayer such as third lipid bilayer 26 which is covalently bonded to second lipid bilayer 22. In a refinement, multilamellar liposome 12 includes on average from 2 to 10 lipid bilayers. In another refinement, multilamellar liposome 12 includes on average from 3 to 9 lipid bilayers. In still another refinement, multilamellar liposome 12 includes on average from 3 to 6 lipid bilayers. Targeting peptide(s) 42 are covalently bonded to the exterior surface of the multilamellar liposome. The present embodiment is not limited by the nature of the covalent bonding which can include bonding via a thioether bond to a maleimide headgroup or a bond to a DTT group attached to the exterior surface of the liposome (e.g., attached to a maleimide headgroup). At least one anticancer compound 28 is disposed within multilamellar liposome 12. In one refinement, cancer compound 28 is disposed within liposomal cavity 18. In another refinement, cancer compound 28 is disposed within the lipid bilayers, for example lipid bilayers 20, 22 and any additional lipid layers. In still other refinements, cancer compound 28 is disposed within liposomal cavity 18 and the lipid bilayers. In some variations, poly(ethylene glycol) groups 30 are covalently bonded to the exterior surface of the liposome in order to improve water solubility. In a refinement, the poly(ethylene glycol) groups have a weight average molecular weight from about 400 to 2500 Daltons. In another refinement the poly(ethylene glycol) groups include from 9 to 45 repeat units of —OCH₂CH₂—.

With reference to FIG. 2A, a targeting peptide that is advantageously used to deliver the anticancer compounds to cancer cells. In some variations, the targeting peptide is any peptide having a corresponding cognate ligand found on or associated with the tumor site. As used herein, the “tumor site” refers to a tumor, tumor cells, tumor tissues, and/or tumor vasculature. The targeting peptide may be any length or size which does not inhibit its targeting and association with at least one corresponding receptor at the tumor site. In a refinement, the target peptide 42 includes the following sequence:

SEQ ID NO 1:
X₁-X₂-X₃-X₄-X₅-X₆-X₇-X₈-X₉

wherein X₁-X₉ are absent or amino acid residues with the proviso that at least one sequence in X₁-X₉ is RRL, RGD, CGGRRLGGC, or CRGDKGPDC. Therefore, the targeting peptide includes at least a 3 amino acid sequence—RRL or RGD. In a refinement, the targeting peptide includes SEQ ID NO 2 which forms a ring structure is forms a ring structure via disulfide bonds as depicted in formula 1

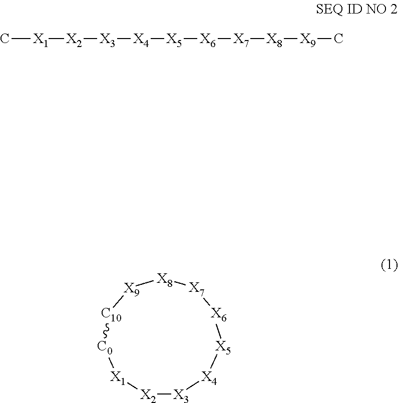

wherein C₀ and C₁₀ are each independently cysteine, a wavy line is a disulfide bond, and X₁-X₉ are absent or an amino acid residues with the proviso that at least one sequence in X₁-X₉ is RRL, RGD, GGRRLGG, or RGDKGPD. In another refinement, the targeting peptide includes a cyclized polypeptide having formula 2:

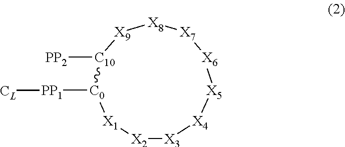

wherein C_{L} is a cysteine that bonds to the liposomes set forth above via a thioether bond, C₀ and C₁₀ are each independently cysteine, PP₁ and PP₂ are each independently absent or an arbitrary polypeptide having from 1 to 10 amino acid residues; and X₁-X₉ are each independently absent or an amino acid residues with the proviso that at least one sequence in X₁-X₉ is RRL, RGD, GGRRLGG, or RGDKGPD. In a refinement, X₁-X₉ are RRL, RGD, GGRRLGG, or RGDKGPD. In another refinement, PP₁ and PP₂ are each independently absent or an arbitrary polypeptide having from 1 to 5 amino acid residues. In another refinement, PP₁ and PP₂ are each independently absent or an arbitrary polypeptide having from 1 to 3 amino acid residues. Circularization of the targeting peptides may be carried out using any suitable method. Circularization methods are known in the art as described, e.g., in Davies, 2003, J. of Peptide Science, 9:471-501, the entire contents of which are herein incorporated by reference.

In some variations, the targeting peptide targets and binds to a cognate moiety on a tumor. The cognate moiety is a biomolecule that has an affinity for and interacts with a targeting peptide. As used herein, a cognate moiety may be referred to as a receptor in that it receives and interacts (e.g., by binding) to the targeting peptide, but the cognate moiety does not necessarily function as a receptor at the tumor site in the absence of the targeting peptide. Additionally, the cognate moiety does not have to be isolated or identified. That is, the cognate moiety may interact with, or bind to, a particular targeting peptide, and the cognate moiety may be associated with a particular tumor site, but the molecular details (e.g., the amino acid sequence) of the cognate moiety do not necessarily have to be known.

Furthermore, a cognate moiety may be specific to a tumor site, but a targeting peptide that associates with, or binds to, a cognate moiety that is overexpressed at a tumor site (while having a low level or no expression on normal cells) could also be useful for targeting a CML carrying a therapeutic load. For example, the ROD peptide targets tumor sites by binding to α_{υ}β₃ and α_{υ}β₅ integrins which are highly expressed in tumor endothelium, as described in Sugahara et al., 2009, Cancer Cell, 16:510-520; Mitra et al., 2005, J. of Controlled Release, 102:191-201; and Murphy et al., 2008, PNAS, 105:9343-9348, the entire contents of all of which are herein incorporated by reference. In another example, the RRL peptide targets tumor vasculature on tumor derived endothelium (TDECs), as described in Brown et al., 2000, Annals of Surg. Oncol., 7:743-749; Weller et al., 2005, Cancer Res., 65:533-539; and U.S. Pat. No. 6,974,791 to Wong et al., the entire contents of all of which are herein incorporated by reference.

With reference to FIG. 2B, a schematic flowchart illustrating the preparation of the multilamellar liposomes of FIG. 2A is provided. This method is adapted from Moon et al., (2011) Interbilayer-crosslinked Multilamellar Vesicles as Synthetic Vaccines for Potent Humoral and Cellular Immune Responses; Nat. Mater. 10, 243-251; the entire disclosure of this publication is hereby incorporated by reference. This preparation is based on the conventional dehydration-rehydration method. In general, the liposomes are formed through covalently crosslinking functionalized headgroups of adjacent lipid bilayers and in particular the incorporation of a thiol-reactive maleimide headgroup lipid (e.g., N-(3-Maleimide-1-oxopropyl)-L-α-phosphatidylethanolamine, MPB-PE) onto the surface of unilamellar liposome 32. In step a), divalent cation-triggered vesicle fusion yields a multilamellar structure 34. In step b), interbilayer crosslinking across the opposing sides of lipid bilayers through the reactive headgroups with dithiothreitol (DTT) generates robust and stable crosslinked multilamellar liposomes 36. In step c), targeting peptides 48 are conjugated to the surface of liposomes 36 through the functional thiol-reactive maleimide headgroups of maleimide-headgroup lipid (e.g., 1,2-dioleoyl-sn-glycero-3-phosphoeth-anolamine-N-[4-(p-maleimidophenyl)butyramide (MPB-PE)) to form targeted multilamellar liposomes 50. In step d, PEG groups 38 are added to the surface of the crosslinked multilamellar liposome 36 with a thiol-terminated PEG to form crosslinked multilamellar liposomes 52.

As depicted in FIGS. 1A, 1B, 2A, and 2B, the lipid bilayers such as first lipid bilayer 20 and second lipid bilayer 22 and any additional lipid bilayers each independently include lipids with maleimide-headgroups M which are bonded together by covalent bond group 24. In particular, the lipid bilayers each independently include a maleimide-containing diacylglycerol lipid. Examples of maleimide-containing diacylglycerols include, but are not limited to, a sodium salt of a compound elected from the group consisting of 1,2-dioleoyl-sn-glycero-3-phosphoethanolamine-N-[4-(p-maleimidomethyl)cyclohexane-carboxamide], 1,2-dioleoyl-sn-glycero-3-phosphoethanolamine-N-[4-(p-maleimidophenyl)butyramide], 1,2-dipalmitoyl-sn-glycero-3-phosphoethanolamine-N-[4-(p-maleimidomethyl)cyclohexane-carboxamide], and 1,2-dipalmitoyl-sn-glycero-3-phosphoethanolamine-N-[4-(p-maleimidophenyl)butyramide], and combinations thereof. As depicted in FIGS. 1A and 1B maleimide headgroups M on adjacent lipid bilayers are covalently bonded together by linking group L. In a refinement, L is an optionally substituted —(CH₂)ₙ—, an optionally substituted —S(CH₂)ₙS—, an optionally substituted —O(CH₂)ₙO—, and the like. In particular, when L is dithiothreitol, L is —S(CHOH)(CHOH)S—.

Typically, the lipid bilayers such as first lipid bilayer 20 and second lipid bilayer 22 and any additional lipid bilayers additional lipids and in particular phospholipids that are different than the lipids with maleimide-headgroups. For example, the lipid bilayers each independently include a phospholipid that is a fatty acid di-ester of phosphatidylcholine, ethylphosphatidylcholine, phosphatidylglycerol, phosphatidic acid, phosphatidylethanolamine, phosphatidylserine or sphingomyelin. In a refinement, the weight ratio of lipids with maleimide-headgroups to lipids that do not include such headgroups is from 3:5 to 5:3. In another refinement, the weight ratio of lipids with maleimide-headgroups to lipids that do not include such headgroups is from 4:5 to 5:4.

As set forth above, the compositions of the present invention include at least one anticancer compound. Examples of anticancer compounds include, but are not limited to a component selected from the group consisting of a DNA alkylating agent, oxidant, topoisomerase inhibitor, and combinations thereof. Specific anticancer compounds include, but are not limited to, methyl methanesulfonate, cyclophosphamide, etoposide, doxorubicin, Taxol (paclitaxel), menadione, taxotere (docetaxel), rapamycin, carboplatinum, cisplatinum, gemcitabine, doxorubicin, siRNAs, and combinations thereof. In particular, examples of anticancer compounds include, but are not limited to, a component selected from the group consisting of doxorubicin and taxol.

The amount of anticancer compounds is variable over a wide range. For example, the crosslinked multilamellar liposomes can include from 100 to 300 mg of anticancer compounds to gram of lipids. In a refinement, the crosslinked multilamellar liposomes include from 200 to 300 mg of anticancer compounds to gram of lipids. In another refinement, the crosslinked multilamellar liposomes include from 250 to 300 mg of anticancer compounds to gram of lipids.

In variations of the embodiments of FIGS. 1A, 1B, 2A, and 2B, a refinement, a first and second anticancer compound is disposed within multilamellar liposomes 10 or 10′. In one refinement, the first compound is hydrophobic and disposed with the lipid bilayer. In another refinement, the second compound is hydrophilic and disposed with the central liposomal cavity 18. Examples of hydrophobic chemotherapeutic compounds include, but are not limited to, Taxol (paclitaxel), taxotere (docetaxel), rapamycin, etc., and combinations thereof. Examples of hydrophilic chemotherapeutic compounds include, but are not limited to, carboplatinum, cisplatinum, gemcitabine, doxorubicin, siRNAs, etc., and combinations thereof. Examples of combinations to be incorporated into the liposomes include, but are not limited to, Taxol and carboplatinum; Taxol and cisplatinum; taxotere and gemcitabine, and taxol and gemcitabine.

In another variation, the anti-cancer compounds include at least one siRNA. In a refinement, the anti-cancer compounds include at least two siRNAs (i.e, 2, 3, 4, 5 or more siRNAs). In a particularly useful refinement, the siRNA is directed to an androgen receptor. Specific examples of siRNA directed to the androgen receptor are:

SEQ ID NO 3:
rCrUrArCrArArCrUrUrUrCrCrArCrUrGrGrCrUTT
(forward sequence)
SEQ ID NO 4:
rArGrCrCrArGrUrGrGrArArArGrUrUrGrUrArGTT
(reverse sequence for SEQ ID NO 3)
SEQ ID NO 5:
rGrCrUrGrCrArArGrGrUrCrUrUrCrUrUrCrArATT
(forward sequence)
SEQ ID NO 6:
rUrUrGrArArGrArArGrArCrCrUrUrGrCrArGrCTT
(reverse sequence for SEQ ID NO 5)
SEQ ID NO 7:
rGrCrArGrArArArUrGrArUrUrGrCrArCrUrArUTT
(forward sequence)
SEQ ID NO 8:
rArUrArGrUrGrCrArArUrCrArUrUrUrCrUrGrCTT
(reverse sequence for SEQ ID NO 7)
SEQ ID NO 9:
rGrCrUrGrArArGrArArArCrUrUrGrGrUrArArUTT
(forward sequence)
SEQ ID NO 10:
rArUrUrArCrCrArArGrUrUrUrCrUrUrCrArGrCTT
(reverse sequence for SEQ ID NO 9)

In other variations of the embodiments of FIGS. 1A, 1B, 2A, and 2B, the liposome compositions further include a pharmaceutically acceptable aqueous carrier such as an aqueous buffer.

It should be appreciated that variations of the crosslinked multilamellar liposomes compositions are a nanoparticle delivery system. In this regard, the crosslinked multilamellar liposomes typically have an average diameter less than about, in increasing order of preference, 600 nm, 500 nm, 400 nm, 300 nm, and 250 nm. In yet other refinements, the crosslinked multilamellar liposomes have an average diameter greater than about, in increasing order of preference, 60 nm, 70 nm, 80 nm, 90 nm, and 100 nm. Typically, the crosslinked multilamellar liposomes have an average diameter 220+/−14.99 nm.

In another embodiment, a method for treating or alleviating a symptom of cancer is provided. The method includes a step of identifying a subject having cancer or exhibiting a symptom of cancer. A therapeutically effective amount of a liposome composition is administered to the subject. The liposome compositions are the liposome compositions set forth above in connection to the descriptions of FIGS. 1A, 1B, 2A, and 2B. In particular, the composition includes a crosslinked multilamellar liposome having an exterior surface and an interior surface, the interior surface defining a central liposomal cavity. The multilamellar liposome includes at least a first lipid bilayer and a second lipid bilayer. The first lipid bilayer are covalently bonded to the second lipid bilayer. At least one anticancer compound is disposed within the crosslinked multilamellar liposome. In a particularly useful composition, a targeting peptide covalently bonded to the exterior surface of the multilamellar liposome as set forth above in connection to the description of FIGS. 2A and 2B. The method of the present embodiment is particularly useful for treating breast cancer, prostate cancer, cervical cancer, and melanoma.

In a variation of the present embodiment, the liposome composition includes a combination of chemotherapeutic compounds in predetermined ratios. Advantageously, the crosslinked multilamellar liposomes are able to provide ratiometric control of the chemotherapeutic compounds disposed therein when the liposomes are delivered a site of interest, i.e., a cancer or tumor cell. In particular, the compositions disclosed herein are able to prolong drug circulation time, reduce systemic toxicity, and increase drug accumulation at tumor sites through the enhanced permeability and retention (EPR) effect. Moreover, the liposome compositions coordinate the plasma elimination and biodistribution of multiple drugs, enabling dosage optimization to maximize cytotoxicity while minimizing the chances to develop drug resistance. Compared to other nanoparticle delivery systems, the crosslinked liposomes provide superior ability to co-deliver multiple drugs with vastly different hydrophobicities to the same site of action while possessing superior stability as compared to prior art liposome formulations. The crosslinked multilamellar liposomes are capable of prolonging maintenance of the synergistic ratios of combined drugs in vivo and, in turn, providing a significantly enhanced antitumor efficacy compared to free-drug cocktail administration.

In one refinement, the combination of chemotherapeutic compounds includes at least one hydrophobic compound and at least one hydrophilic compound in a predetermined ratio. In a further refinement, the liposomes include two or more hydrophobic compound and/or two or more hydrophilic compounds. Examples of hydrophobic chemotherapeutic compounds include, but are not limited to, Taxol (paclitaxel), taxotere (docetaxel), rapamycin, etc., and combinations thereof. Examples of hydrophilic chemotherapeutic compounds include, but are not limited to, carboplatinum, cisplatinum, gemcitabine, doxorubicin, siRNAs, etc., and combinations thereof. Examples of combinations to be incorporated into the liposomes include, but are not limited to, Taxol and carboplatinum; Taxol and cisplatinum; taxotere and gemcitabine, and taxol and gemcitabine. A particularly useful combination is doxorubicin and paclitaxel. In a refinement, the weight ratio of the hydrophilic compound to the hydrophobic compound is from about 1:5 to 5:1. In another refinement, the weight ratio of the hydrophilic compound to the hydrophobic compound is from about 3:3 to 5:1. In a more specific refinement, the weight ratio of doxorubicin to paclitaxel is from about 1:5 to 5:1. In a particularly useful refinement, the weight ratio of doxorubicin to paclitaxel is from about 3:3 to 5:1. The combination of doxorubicin and paclitaxel is useful for the treatment of metastatic breast cancer and melanoma. The combination of doxorubicin and paclitaxel in a crosslinked multilamellar liposome exhibits a synergistic effect at weight ratios of doxorubicin to paclitaxel is from about 3:3 to 5:1 in a breast tumor model without significant cardiac toxicity.

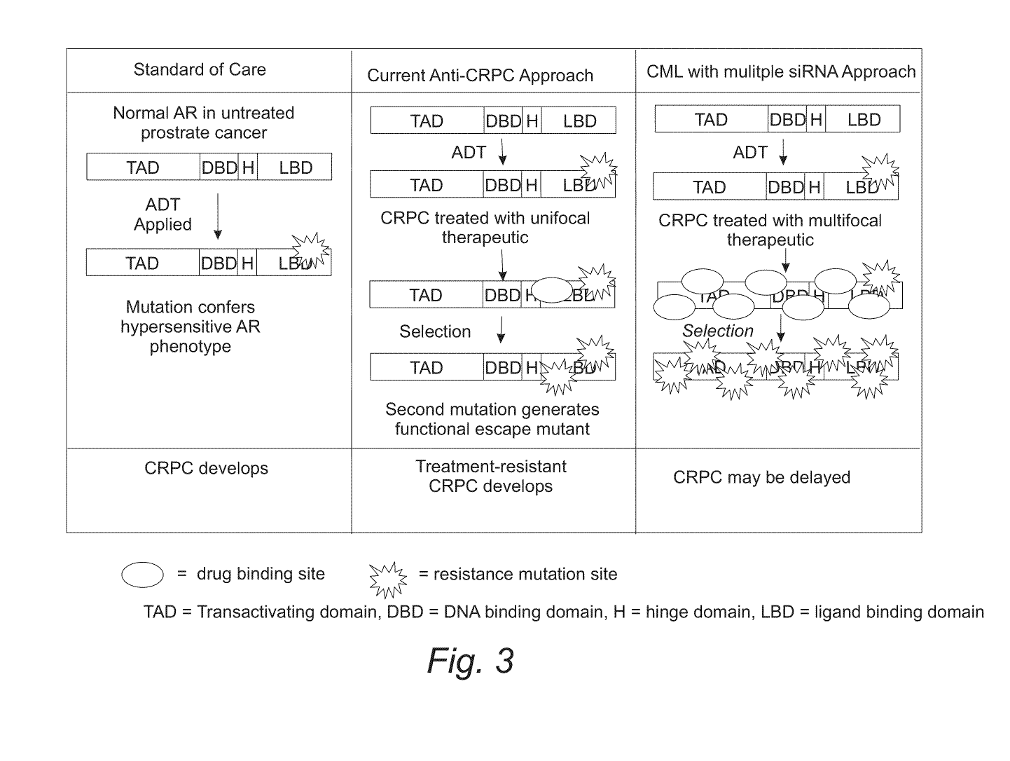

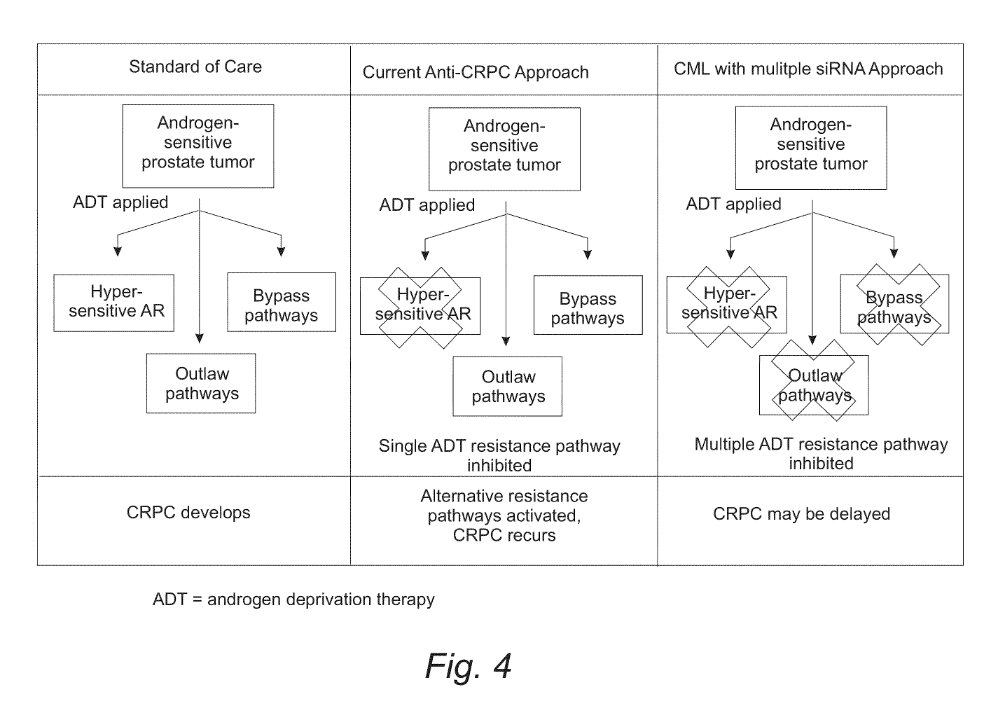

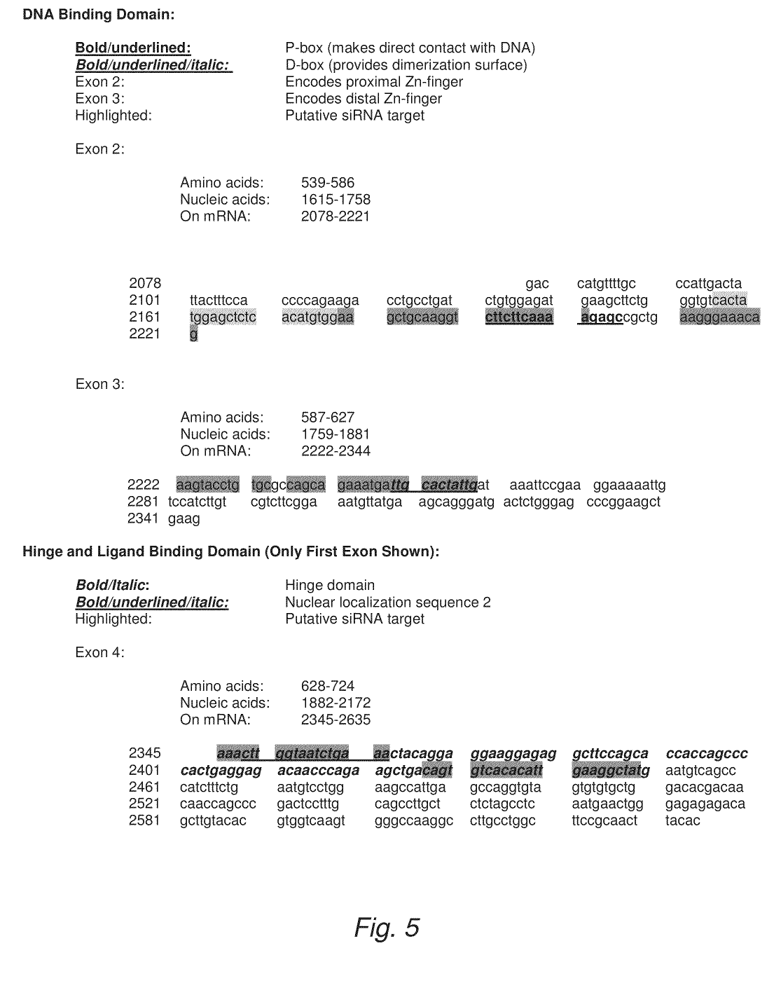

In a variation of the present embodiment, the subject has castration resistant prostate cancer (CRPC). Therefore, such subjects are treated with multilamellar liposomes that include at least two siRNAs that target multiple sites that are critical to androgen receptor function. FIGS. 3 and 4 provide schematic illustrations comparing prior art treatment modes versus the method of the present embodiment. In the present variation, multiple siRNAs are complementary to mRNA sequences that encode critical functional domains of the androgen receptor. Coding sequences of escape mutants are therefore likely to be so divergent from wild type that they no longer encode a functional androgen receptor. FIG. 5 provides potential targets of the androgen receptor for the siRNAs. Specific examples include, but are not limited to:

SEQ ID NO 3:
rCrUrArCrArArCrUrUrUrCrCrArCrUrGrGrCrUTT
(forward sequence)
SEQ ID NO 4:
rArGrCrCrArGrUrGrGrArArArGrUrUrGrUrArGTT
(reverse sequence for SEQ ID NO 3)
SEQ ID NO 5:
rGrCrUrGrCrArArGrGrUrCrUrUrCrUrUrCrArATT
(forward sequence)
SEQ ID NO 6:
rUrUrGrArArGrArArGrArCrCrUrUrGrCrArGrCTT
(reverse sequence for SEQ ID NO 5)
SEQ ID NO 7:
rGrCrArGrArArArUrGrArUrUrGrCrArCrUrArUTT
(forward sequence)
SEQ ID NO 8:
rArUrArGrUrGrCrArArUrCrArUrUrUrCrUrGrCTT
(reverse sequence for SEQ ID NO 7)
SEQ ID NO 9:
rGrCrUrGrArArGrArArArCrUrUrGrGrUrArArUTT
(forward sequence)
SEQ ID NO 10:
rArUrUrArCrCrArArGrUrUrUrCrUrUrCrArGrCTT
(reverse sequence for SEQ ID NO 9)

In still another embodiment, a method for treating or alleviating a symptom of cancer is provided. The method includes a step of identifying a subject having or exhibiting a symptom of anticancer drug resistant cancer, and in particular multidrug resistant cancer. In refinement, the existence of multidrug resistance is assessed by determining the expression of P-glycoprotein (P-gp) in cancer cells. This determination can be achieved histologically with P-gp-specific antibody stain. A therapeutically effective amount of a liposome composition is administered to the subject. The liposome compositions are the liposome compositions set forth above in connection to the descriptions of FIGS. 1A, 1B, 2A, and 2B. In particular, the composition includes a crosslinked multilamellar liposome having an exterior surface and an interior surface, the interior surface defining a central liposomal cavity. The multilamellar liposome includes at least a first lipid bilayer and a second lipid bilayer. The first lipid bilayer are covalently bonded to the second lipid bilayer. At least one anticancer compound is disposed within the crosslinked multilamellar liposome. In a particularly useful composition, a targeting peptide covalently bonded to the exterior surface of the multilamellar liposome as set forth above in connection to the description of FIGS. 2A and 2B. The method of the present embodiment is particularly useful for treating breast cancer, prostate cancer, cervical cancer, and melanoma. In a refinement of this embodiment, a combination of chemotherapeutic compounds and in particular a combination of hydrophobic and hydrophilic compounds with the ratios as set forth above is disposed in the liposomes. Once again, combination of doxorubicin and paclitaxel in a crosslinked multilamellar liposome with weight ratios of doxorubicin to paclitaxel from about 1:5 to 5:1, and in particular, from about 3:3 to 5:1 is found to be useful as substantiated by a breast cancer model.

In the methods set forth above, the therapeutically effective amount of the liposome compositions are usually such that the subject receives the amount of the anticancer compounds generally utilized for a specific cancer by standard protocols. For example, the amount of anticancer compounds delivered by the crosslinked liposomes is set to match the dosage regimens of Table 1 which are used in conventional therapies.

TABLE 1

| Anticancer compound | Dosage |
|---|---|
| Cyclophosphamide | 1-5 mg/kg daily; 40-50 mg/kg IV in divided |
| | doses × 2-5 days, 3-5 mg/kg IV 2×/week. |
| Etoposide | Oral 50-100 mg daily × 14 days, |
| | IV: 50-100 mg/m2 IV on days 1-3 or 1,3,5 of |
| | cycle and 70 mg/m2 po on day 1-4 |
| Doxorubicin | 60-75 mg/m2 IV Day 1 every 21-28 days, or 40- |
| | 65 mg/m2 on Day 1 of similar schedule in |
| | combination with other cytotoxic drugs. |
| Taxol (paclitaxel) | 175-225 mg/m² IV every 3 weeks, 135 mg/m2 |
| | IV q 21 days, 80 mg/m2 weekly (3 weeks on, 1 |
| | week off) |
| Doxorubicin | 60-75 mg/m² IV Day 1 every 21-28 days, or |
| | 40-65 mg/m² on Day 1 of similar schedule in |
| | combination with other cytotoxic drugs. |
| Taxol (paclitaxel) | 175-225 mg/m² IV every 3 weeks, 135 mg/m² IV |
| | q 21 days, 80 mg/m² weekly (3 weeks on, 1 |
| | week off) |
| Taxotere | 60-100 mg/m2 IV of a 21 day cycle. 75 mg/m² |
| | IV every 21 days. |
| Carboplatin: | Dosed according to Area Under the Curve: AUC |
| | 2-6 every 21 days. |
| | 300-360 mg/m2 every 28 day cycle. |
| Cisplatin | 20 mg/m2 on Days 1-5 of 21 days cycle (in |
| | combination); 50-100 mg/m2 on day 1 of an 21 |
| | day cycle. |
| Gemcitabine | 675-1250 mg/m2 IV on Days 1, 8 of a |
| | combination 21 day cycle. |
| | 1000 mg/m2 weekly × 3 or 5 total of a 4 or 6 |
| | week cycle. |

The following examples illustrate the various embodiments of the present invention. Those skilled in the art will recognize many variations that are within the spirit of the present invention and scope of the claims.

### 1. Crosslinked Multilamellar Liposomes for Controlled Delivery of Anticancer Drugs

### Materials and Methods

Cell Lines, Antibodies, Reagents, and Mice.

B16 tumor cells (B16-F10, ATCC number: CRL-6475) and HeLa cells were maintained in a 5% CO2 environment with Dulbecco's modified Eagle's medium (DMEM) (Mediatech, Inc., Manassas, Va.) supplemented with 10% FBS (Sigma-Aldrich, St. Louis, Mo.) and 2 mM of L-glutamine (Hyclone Laboratories, Inc., Omaha, Nebr.). The mouse monoclonal antibodies against clathrin, caveolin-1, EEA1, and the rabbit polyclonal antibody specific to trans-Golgi network (TGN38) were purchased from Santa Cruz Biotechnology, Inc. (Santa Cruz, Calif.). The mouse monoclonal antibody to Lamp-1 was purchased from Abcam (Cambridge, Mass.). Alexa488-goat anti-mouse immunoglobulin G (IgG) and Alexa594-goat anti-rabbit IgG antibodies were purchased from Invitrogen (Carlsbad, Calif.). Chloropromazine, Nystatin, and MβCD were obtained from Sigma-Aldrich.

All lipids were obtained from NOF Corporation (Japan): 1,2-dioleoyl-sn-glycero-3-phosphocholine (DOPC), 1,2-dioleoyl-sn-glycero-3-phospho-(1′-rac-glycerol) (DOPG), and 1,2-dioleoyl-sn-glycero-3-phosphoethanolamine-N-[4-(p-maleimidophenyl) butyramide (maleimide-headgroup lipid, MPB-PE). ⁶⁴Cu was obtained from Washington University (St. Louis, Mo.) and the University of Wisconsin (Madison, Wis.). 1-Ethyl-3-[3-dimethylaminopropyl]carbodiimide hydrochloride (EDC) and N-hydroxysulfosuccinimide (SNHS) were purchased from Thermo Scientific (Rockford, Ill.).

Female C57BL/6 mice, 6-10 weeks old, were purchased from Charles River Breeding Laboratories (Wilmington, Mass.). All mice were held under specific pathogen-reduced conditions in the animal facility of the University of Southern California (USA). All experiments were performed in accordance with the guidelines set by the National Institutes of Health and the University of Southern California on the Care and Use of Animals.

Synthesis of CMLs, ULs, and DLLs.

Liposomes were prepared based on the conventional dehydration-rehydration method. 1.5 μmol of lipids of DOPC, DOPG, and MPB-PE at the molar ratio of the lipid composition of DOPC:DOPG:MPB-PE=40:10:50, were mixed in chloroform, and the organic solvent in the lipid mixture was evaporated under argon gas and dried under vacuum overnight to form dried thin lipid films. The resultant dried film was hydrated in 10 mM Bis-Tris propane at pH 7.0 containing doxorubicin at a molar ratio of 0.5:1 (drugs:lipids), with vigorous vortexing every 10 min for 1 h, and then applied with 4 cycles of 15-s sonication (Misonix Microson XL2000, Farmingdale, N.Y.) on ice at 1 min intervals for each cycle. To induce divalent-triggered vesicle fusion, MgCl₂ was added to make a final concentration of 10 mM. The resulting multilamellar vesicles were further crosslinked by addition of Dithiothreitol (DTT, Sigma-Aldrich) at a final concentration of 1.5 mM for 1 h at 37° C. The resulting vesicles were collected by centrifugation at 14,000 g (12,300 RPM) for 4 min and then washed twice with PBS. For pegylation of CMLs, the liposomes were further incubated with 1 μmol of 2 kDa mPEG-SH (Laysan Bio Inc., Arab, Ala.) for 1 h at 37° C. The particles were then centrifuged and washed twice with PBS. Nonencapsulated doxorubicin was removed by a PD-10 Sephadex gel filtration column, and then the final products were stored in PBS at 4° C. Similarly, unilamellar liposomes (ULs) were prepared with the same lipid composition through rehydration, vortexing and sonication, as described above, except divalent-induced vesicle fusion and DTT crosslinking processes. The ULs were collected by centrifugation at 250,000 g for 90 min and then washed twice with PBS. Pegylation of ULs was carried out by incubation with 1 μmol of 2 kDa PEG-SH. Doxil-like liposomes (DLLs) were prepared using an ammonium sulfate pH gradient method as described in Haran G, Cohen R, Bar L, Barenholz Y. Transmembrane ammonium sulfate gradients in liposomes produce efficient and stable entrapment of amphipathic weak bases. Biochim Biophys Acta. 1993; 1151:201-15. Briefly, lipid film (HSPC:Cholesterol:DSPE-PEG₂₀₀₀=56:38:6) was rehydrated with 240 mM of ammonium sulfate buffer pH 5.4 with vigorous vortexing. Small unilamellar vesicles were prepared using sonication and extrusion at 60° C. through 100 nm polycarbonate filters 20 times using a mini-Extruder (Avanti Polar Lipids, Alabaster, Ala.). The DLLs were collected by centrifugation at 250,000 g (45,400 RPM) for 90 min, washed twice with PBS, and then resuspended with HBS pH 7.4 (20 mM HEPES, 150 mM NaCl) containing doxorubicin hydrochloride. The particles were then centrifuged and washed twice with PBS. Nonencapsulated doxorubicin was removed by a PD-10 Sephadex gel filtration column, and then the final products were stored in PBS at 4° C.

Characterization of Physical Properties.

The hydrodynamic size and size distribution of CMLs, ULs and DLLs were measured by dynamic light scattering (Wyatt Technology, Santa Barbara, Calif.). For cryo-electron microscopy imaging, the liposome samples were applied to the grid and plunge-frozen in liquid ethane using the FEI Mark III Vitrobot. CryoEM images were collected using a Tecnai T12 electron microscope (FEI Company) equipped with a Gatan Ultrascan 2k by 2k CCD camera.

In Vitro Drug Encapsulation, Release Kinetics, and Cytotoxicity.

To study the loading capacity of Dox, Dox-loaded CMLs, ULs, or DLLs were collected and then washed twice with PBS, followed by lipid extraction of vesicles with 1% Triton X-100 treatment. Lipid concentrations of liposome suspensions were determined by phosphate assay (Itaya K, Ui M. A new micromethod for the colorimetric determination of inorganic phosphate. Clin Chim Acta. 1966; 14:361-6). Dox fluorescence (excitation 480 nm, emission 590 nm) was then measured by a Shimadzu RF-5301PC spectrofluorometer (Japan). To determine a half-time t_{1/2} whereby 50% of entrapped Dox is released from liposomes, CMLs or ULs were incubated at 37° C. in 10% fetal bovine serum (FBS)-containing media, and the releasing media were collected to measure Dox fluorescence at regular time intervals. To obtain the release kinetics of Dox from liposomes, Dox-loaded CMLs, ULs or DLLs were incubated at 37° C. in 10% fetal bovine serum (FBS)-containing media, the releasing media were removed from CMLs, ULs, or DLLs incubated at 37° C. for quantification of Dox fluorescence every day, and fresh media were replaced for continuous monitoring of drug release.

B16 or HeLa cells were plated at a density of 5×10³ cells per well in D10 media in 96-well plates and grown for 6 h. The cells were then exposed to a series of concentrations of Dox-loaded CMLs, ULs or DLLs for 48 h, and the cytotoxicity of Dox-liposomes was assessed using the Cell Proliferation Kit II (XTT assay) from Roche Applied Science, according to the manufacturer's instructions.

Confocal Imaging.

To label liposome particles with DiD lipophilic dyes, DiD dyes were added to the lipid mixture in chloroform at a ratio of 0.01:1 (DiD:lipids), and the organic solvent in the lipid mixture was evaporated under argon gas to incorporate DiD dyes into a lipid bilayer of vesicles. For the colocalization study with endocytic markers, HeLa cells were seeded on glass bottom dishes (MatTek Corporation, Ashland, Mass.) and grown at 37° C. overnight. The cells were then incubated with DiD-labeled CML particles for 30 min at 4° C. to synchronize internalization. After washing with PBS, the treated cells were then warmed to 37° C. to initiate particle internalization for the indicated time periods. The cells were fixed, permeabilized with 0.1% Triton X-100, and then immunostained with the corresponding antibodies specific to clathrin, caveolin-1, EEA1, TGN38, or Lamp-1 and counterstained with DAPI (Invitrogen).

Fluorescence images were acquired on a Yokogawa spinning-disk confocal scanner system (Solamere Technology Group, Salt Lake City, Utah) using a Nikon eclipse Ti-E microscope equipped with a 60×/1.49 Apo TIRF oil objective and a Cascade II: 512 EMCCD camera (Photometrics, Tucson, Ariz., USA). Image processing and data analysis were carried out using the Nikon NIS-Elements software. To quantify the extent of colocalization, the Manders' overlap coefficients (MOC) were generated using the Nikon NIS-Elements software by viewing more than 50 cells at each time point.

Uptake Inhibition Assay.

HeLa cells (1×10⁵ cells) were pretreated with chlorpromazine (CPZ, 25 μg/ml), nystatin (50 μg/ml), or methyl-β-cyclodextrin (MβCD, 15 mM) to disrupt clathrin- or caveolin-mediated entry pathway. The cells were then incubated with DiD-labeled CMLs for 1 h at 37° C. in the presence of CPZ and nystatin or in the absence of MβCD. The cells were then washed twice with PBS. To disrupt energy-dependent internalization of CMLs, HeLa cells were incubated with DiD-labeled CMLs at 4° C. for 1 h and then washed twice with cold PBS. The cellular uptake of particles was determined by measuring DiD fluorescence using the spectrofluorometer and was normalized based on the fluorescent intensity acquired upon incubation at 37° C. for 1 h.

Maximum Tolerated Dose Study.

C57BL/6 female mice (6-10 weeks old) were administered by a single intravenous injection through tail vein with CML-Dox or free Dox at doses of 0, 20 and 40 mg/kg Dox equivalents. The mice were weighed and monitored daily for 8 days after injection.

In Vivo Tumor Challenge.

C57BL/6 female mice (6-10 weeks old) were inoculated subcutaneously with 1×10⁶ B16 melanoma tumor cells. The tumors were allowed to grow for 6 days to a volume of 50˜100 mm³ before treatment. On day 6, the mice were injected intravenously through tail vein with CML-Dox, UL-Dox, or DLL-Dox at a dose of 1 or 4 mg/kg Dox equivalent every other day (six mice per group), and tumor growth and body weight were then monitored for an additional 10 days by the end of the experiment. The length and width of the tumor masses were measured with a fine caliper every other day after Dox-liposome injection. Tumor volume was expressed as ½×(length×width²).

In Vivo PET Imaging and Biodistribution.

For radiolabeling liposomes, amine-terminated PEG-SH was used for PEGylation of ULs and CMLs, while DSPE-PEG-NH₂ was used for PEGylation of DLLs, in order to introduce amine groups onto liposomes for further reaction. Unless noted otherwise, all chemicals were analytic grade from Sigma-Aldrich (St. Louis, Mo.). ⁶⁴Cu was produced using the ⁶⁴Ni(p,n)⁶⁴Cu nuclear reaction and supplied in high specific activity as ⁶⁴CuCl₂ in 0.1 N HCl. The bifunctional chelator AmBaSar was synthesized as reported (Cai H, Li Z, Huang C W, Park R, Shahinian A H, Conti P S. An improved synthesis and biological evaluation of a new cage-like bifunctional chelator, 4-((8-amino-3,6,10,13,16,19-hexaazabicyclo [6.6.6]icosane-1-ylamino)methyl)benzoic acid, for 64Cu radiopharmaceuticals. Nucl Med Biol. 2010; 37:57-65). AmBaSar was activated by EDC and SNHS. Typically, 5 mg of AmBaSar (11.1 μmol) in 100 μL water and 1.9 mg of EDC (10 μmol) in 100 μL water were mixed together, and 0.1 N NaOH (150 μL) was added to adjust the pH to 4.0. SNHS (1.9 mg, 8.8 μmol) was then added to the stirring mixture on ice-bath, and 0.1 N NaOH was added to finalize the pH to 4.0. The reaction remained at 4° C. for 30 min. The theoretical concentration of active ester AmBaSar-OSSu was calculated to be 8.8 μmol. Then, 5-20 times AmBaSar-OSSu (based on molar ratios) were loaded to the liposomes of interest. The pH was adjusted to 8.5 using borate buffer (1M, pH 8.5). The reaction remained at 4° C. overnight, after which the size-exclusion PD-10 column was employed to afford the AmBaSar-conjugated liposomes in PBS buffer. AmBaSar-liposome was labeled with ⁶⁴Cu by addition of 1-5 mCi of ⁶⁴Cu (50-100 μg AmBaSar-liposome per mCi ⁶⁴Cu) in 0.1 N phosphate buffer (pH 7.5), followed by 45 min incubation at 40° C. ⁶⁴ Cu-AmBaSar-liposome was purified on a size exclusion PD-10 column using PBS as the elution solvent. Positron emission tomography (PET) imaging of the mice was performed using a microPET R4 rodent model scanner (Concorde Microsystems, Knoxville, Tenn.). The B16-F10 tumor-bearing C57/BL6 mice were imaged in the prone position in the microPET scanner. The mice were injected with approximately 100 μCi ⁶⁴Cu-AmBaSar-liposome via the tail vein. For imaging, the mice were anaesthetized with 2% isoflurane and placed near the center of the field of view (FOV), where the highest resolution and sensitivity are obtained. Static scans were obtained at 1, 3, and 24 h post-injection. The images were reconstructed by a two-dimensional ordered subsets expectation maximum (2D-OSEM) algorithm. Time activity curves (TAC) of selected tissues were obtained by drawing regions of interest (ROI) over the tissue area. The counts per pixel/min obtained from the ROI were converted to counts per ml/min by using a calibration constant obtained from scanning a cylinder phantom in the microPET scanner. The ROI counts per ml/min were converted to counts per g/min, assuming a tissue density of 1 g/ml, and divided by the injected dose to obtain an image based on ROI-derived percent injected dose of ⁶⁴Cu tracer retained per gram (% ID/g). For biodistribution, animals were sacrificed 24 h post-injection; tissues and organs of interest were harvested and weighed. Radioactivity in each organ was measured using a gamma counter, and radioactivity uptake was expressed as percent injected dose per gram (% ID/g). Mean uptake (% ID/g) for each group of animals was calculated.

Pharmacokinetics and Quantification of Dox in Tumors.

C57/BL6 mice bearing B16 tumors (diameter 0.5-1 cm) were injected with free Dox, UL-Dox, DLL-Dox, or CML-Dox at a dose of 10 mg/kg Dox equivalent. To examine pharmacokinetics, blood was collected by retro-orbital bleeding at the indicated time points, and then plasma was obtained by centrifuging the samples at 14,000 g for 10 min. To detect Dox in tumors, the tumors were collected at 24 h after injection and were then homogenized. Dox was extracted by adding methanol to the homogenized samples, followed by vortexing and freeze/thaw cycles. After the extraction of Dox with further purification using an Amicon Ultra 10,000 MWCO centrifugal filter, Dox concentration was quantified by reverse phase HPLC using a C18 column.

### Results

Preparation and Characterization of Crosslinked Multilamellar Liposomal Doxorubicin.

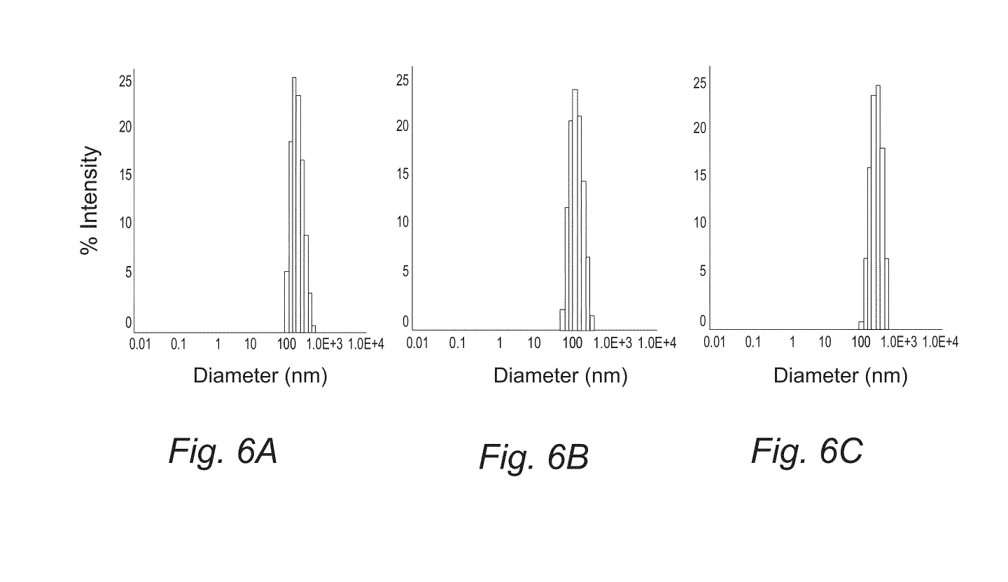

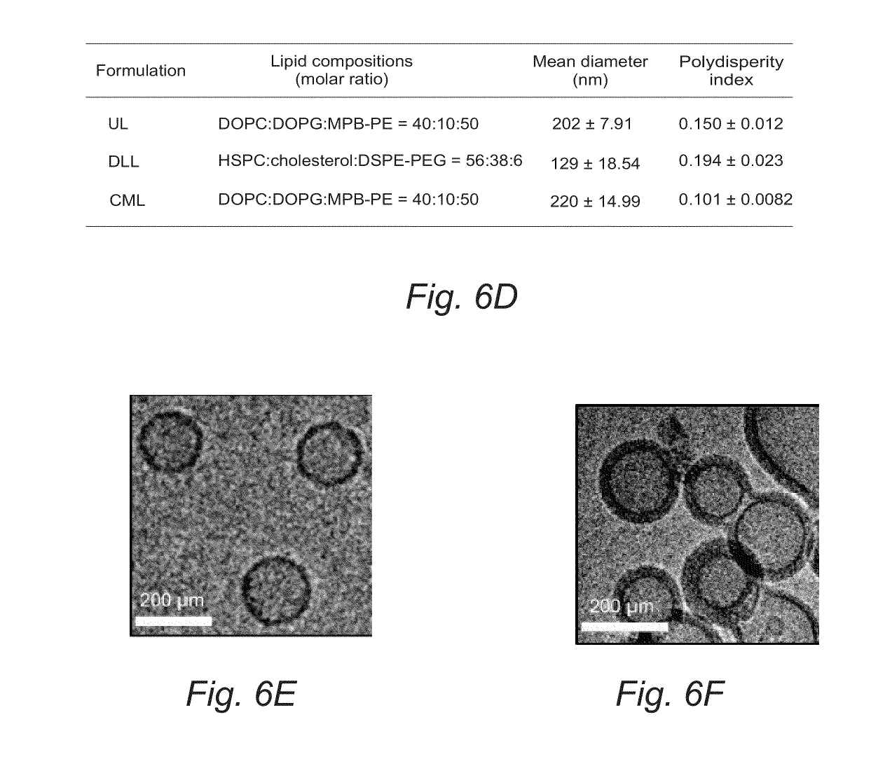

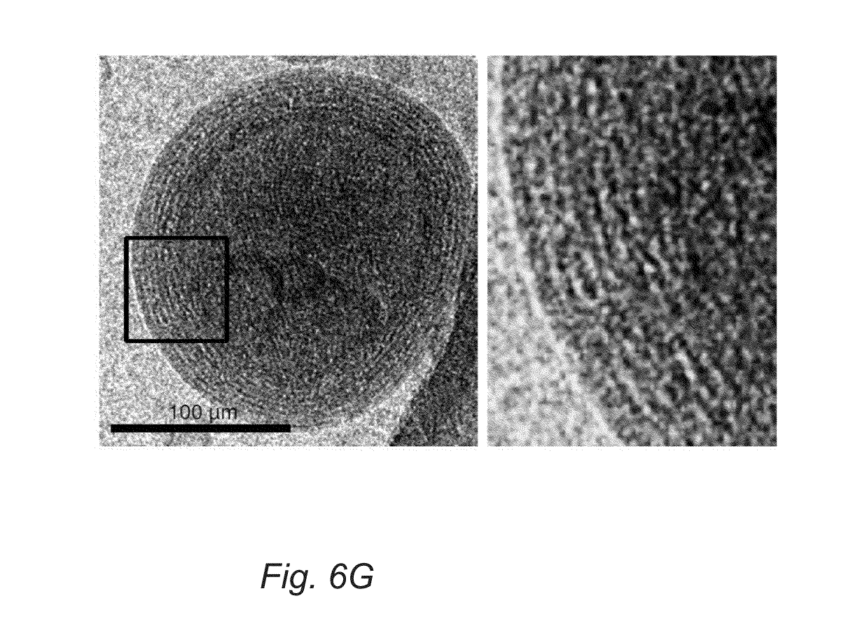

Our goal was to generate a liposomal formulation with improved bioavailability of liposomal drugs and enhanced vesicle stability. To accomplish this, multilamellar vesicles were formed through covalently crosslinking functionalized headgroups of adjacent lipid bilayers, as illustrated in FIG. 1. This design was adapted from a recently reported multistep procedure based on the conventional dehydration-rehydration method (Moon J J, Suh H, Bershteyn A, Stephan M T, Liu H P, Huang B, et al. Interbilayer-crosslinked multilamellar vesicles as synthetic vaccines for potent humoral and cellular immune responses. Nat Mater. 2011; 10:243-51): (1) the incorporation of a thiol-reactive maleimide headgroup lipid (N-(3-Maleimide-1-oxopropyl)-L-α-phosphatidylethanolamine, MPB-PE) onto the surface of unilamellar liposome (UL); (2) divalent cation-triggered vesicle fusion that yields a multilamellar structure; and (3) interbilayer crosslinking across the opposing sides of lipid bilayers through the reactive headgroups with dithiothreitol (DTT) to generate robust and stable vesicles. As a final step, the surface of the crosslinked multilamellar liposome (CML) was PEGylated with thiol-terminated PEG, which could further improve vesicle stability and blood circulation half-life. Additionally, liposomes of approximately the same size and composition as Doxil, termed Doxil-like liposome (DLL), were also prepared for comparison (FIG. 6D). First, we characterized the physical properties of CML compared to those of non-crosslinked UL with the same lipid composition or conventional liposome formulation DLL. The hydrodynamic size of the particles was measured by dynamic light scattering (DLS), and the result showed a slight increase in the mean diameter of CML (˜220 nm) compared to that of the UL (˜200 nm) (FIGS. 6A and 6B), whereas the size of DLL was much smaller (˜129 nm), as expected. It also indicated that CML particles exhibited a narrow size distribution (polydispersity: 0.101±0.0082, FIG. 6C), suggesting no significant aggregation of particles during the crosslinking process. In addition, the CML particles are remarkably stable and can be stored in PBS over two weeks at 4° C. without significant change in size or size distribution (data not shown).

To further confirm the multilamellar structure of liposomes, CML particles were imaged by cryo-electron microscopy. ULs were utilized as a control. The images demonstrated that the CML exhibited multilayered vesicle formation with thick walls (FIG. 6F and Supplementary FIG. 6G), while only a single thin-layer of lipid membrane was found in ULs (FIG. 6E), suggesting that the covalent linkage between adjacent bilayers could lead to a stable multilamellar structure of vesicles. In addition, the mean diameter of CML estimated by cryo-electron microscopy was ˜160 nm.

In Vitro Drug Encapsulation, Release Kinetics, and Cytotoxicity.

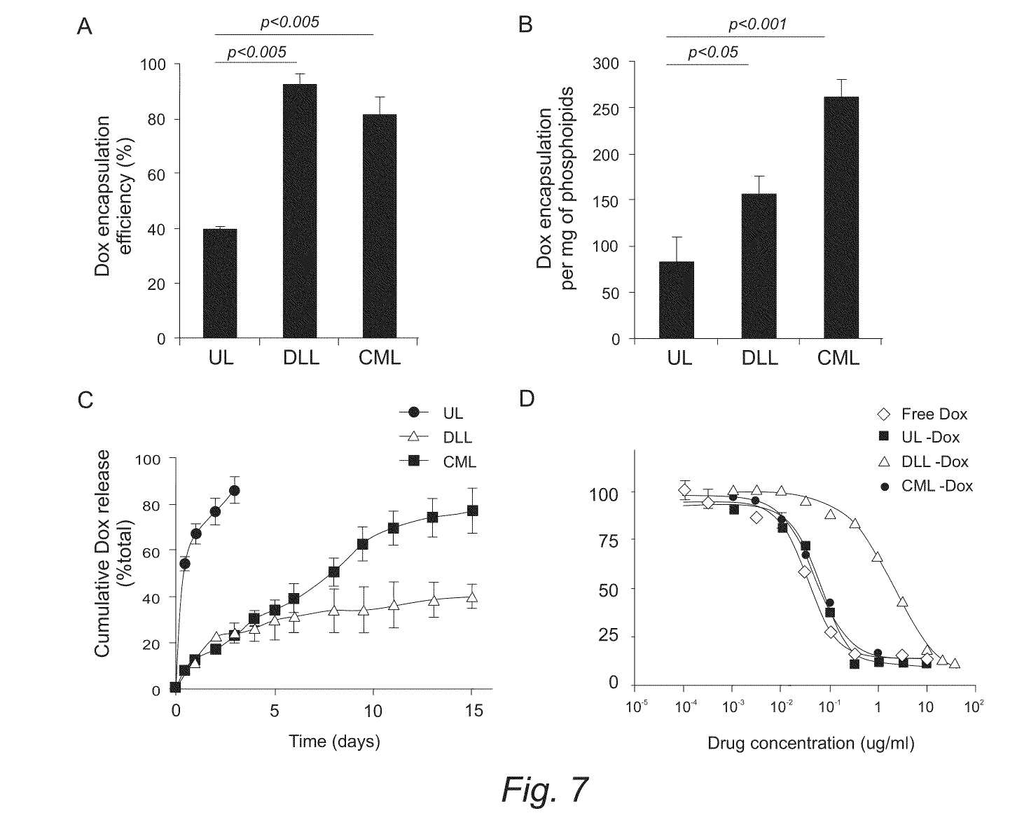

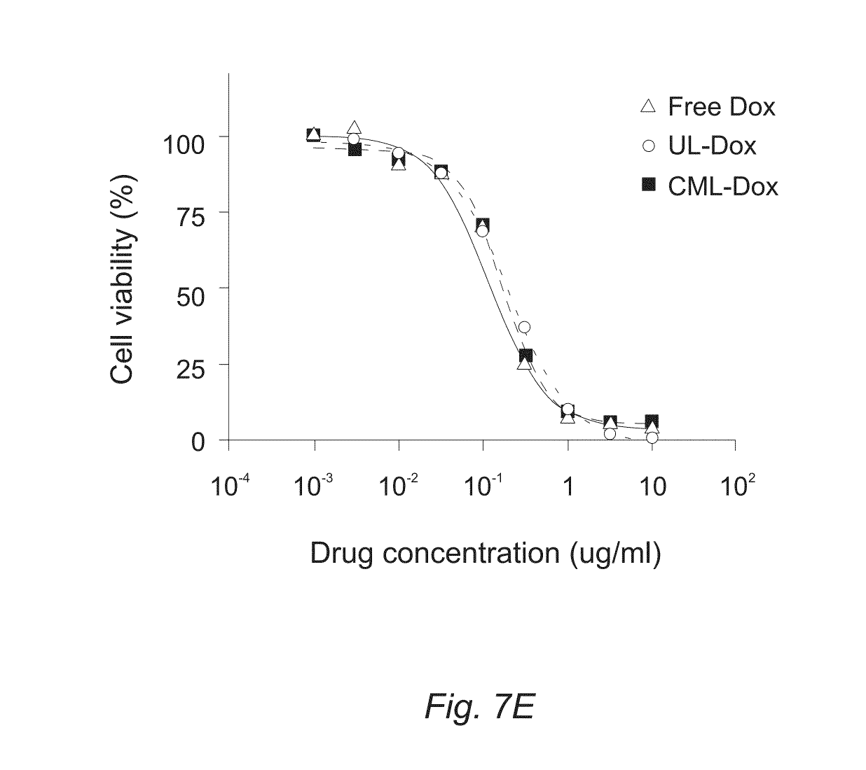

Next, we examined whether the multilamellar structure of CML could improve the loading capacity of the anticancer agent doxorubicin (Dox) into vesicles compared to that of the unilamellar liposomal formulation (FIG. 7A). To test its ability to encapsulate drugs, the lipid film was rehydrated in Dox-containing buffer to form Dox-loaded UL or CML. The result showed that the CML formulation could achieve higher Dox-encapsulation efficiency (˜85%) than that of the UL (˜39.7%) and that the amount of Dox loaded in CML (˜250 μg per mg of lipids) was increased by ˜4-fold compared with UL (FIG. 7B), which was also higher loading efficiency than that of DLL (˜160 μg per mg of lipids). Taken together, these results suggest that the formation of multilamellar structure via vesicle fusion apparently fosters the entrapment of extra drug payload into liposomes.

It is well known that lipid vesicles are exceedingly unstable in the presence of serum, thus limiting their utility as a drug carrier. Serum components disrupt liposome membranes, which causes leakage of their aqueous contents. As an anticancer drug carrier, the stability of liposomal formulations has been intrinsically linked to both toxicity level and therapeutic activity of the drug payload. Therefore, we investigated the vesicle stability of CML in vitro upon exposure to a serum environment relative to the controlled release of its contents. Dox-loaded ULs, DLLs, and CMLs were stored at 37° C. in 10% FBS-containing media, and in vitro drug release rates were measured. As shown in FIG. 7C, ULs had the expected burst release (most released within 2 days), whereas slower and linearly sustained release kinetics (up to two weeks) was seen for CMLs, indicating that the CML formulation could improve vesicle stability in the presence of serum components by forming a crosslinked multilamellar structure. Although significantly slower release kinetics was also observed in DLLs, less than 40% of encapsulated Dox was released from DLLs at 37° C. in two weeks, while CMLs could release ˜80% of encapsulated Dox in two weeks, suggesting that CML formulation could remarkably improve drug release compared with DLLs.

Next, we determined whether this sustained and improved drug release profile of CML could affect cytotoxicity in cells as compared to that of the UL and DLL. Free Dox or Dox-loaded ULs, DLLs, or CMLs were incubated with B16 cells for 48 h, and the cytotoxicity of Dox-liposomes was then measured by a standard XTT assay. In vitro cytotoxicity data revealed that the half-maximal response (EC₅₀) for CMLs was ˜0.05 μg/ml for B16 cells, similar to that of free Dox and ULs (FIG. 7D), suggesting that CMLs were able to maintain Dox cytotoxicity in cells, notwithstanding sustained drug release of the CML formulation. A similar result was also observed in HeLa cells (FIG. 7E). Furthermore, DLLs exhibited higher EC₅₀, 2.3 μg/ml (FIG. 7D), which is consistent with previous reports indicating that Doxil has an EC₅₀ about two orders of magnitude higher (lower cytotoxic activity) than free Dox, suggesting that improved drug release of CML formulation could augment cytotoxicity of liposomal drug, which is likely a result of enhanced uptake and intracellular delivery of Dox to the cells.

Cellular Uptake, Internalization Pathway, and Intracellular Trafficking of CML.

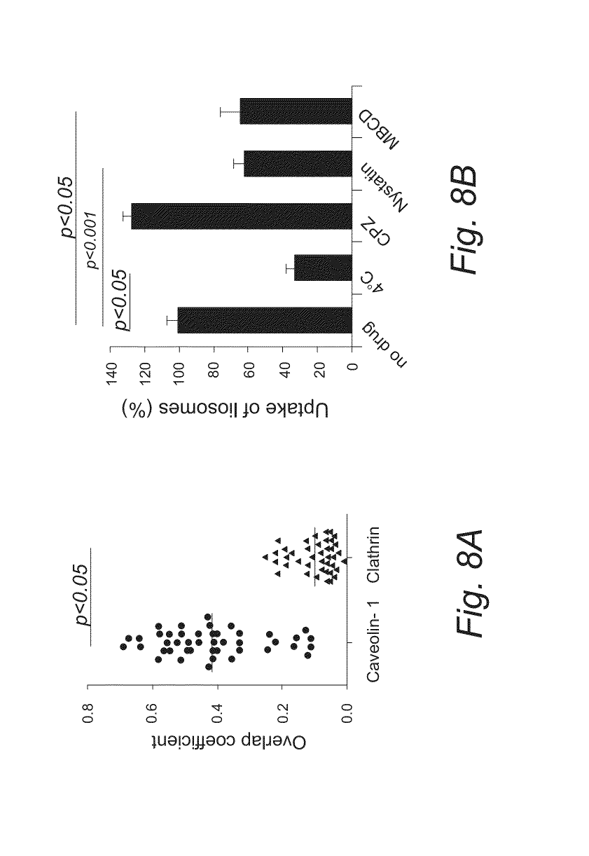

Endocytosis is generally considered one of the main entry mechanisms for various drug nanocarriers. Several endocytic pathways, including clathrin- and caveolin-mediated endocytosis, have been characterized as major routes for cell internalization. Since the intracellular fate of nanocarriers is determined by the endocytic pathways utilized upon their entry to cells, elucidating the fundamental basis of intracellular processing of drug carriers can provide crucial insights for improving the efficiency of drug delivery and developing designs of drug carriers. Hence, to accomplish this, we focused on unraveling the entry mechanism and the subsequent intracellular trafficking of CMLs by visualizing fluorescent 1,1-dioctadecyl-3,3,3,3-tetramethylindodicarbocyanine (DiD)-labeled CML particles and endocytic structures, i.e., clathrin and caveolin, in HeLa cells after 15 min incubation at 37° C. A significant colocalization of CML particles with discrete caveolin-1 signals was observed, whereas, no remarkable colocalization between CMLs and clathrin was detected after 15 min incubation, even though some particles were overlaid with clathrin structures. The quantification of CML particles colocalized with caveolin-1 or clathrin structures by analyzing more than 50 cells suggested that the caveolin-mediated pathway might be involved in the entry of CMLs (FIG. 8B). Involvement of the caveolin pathway was further confirmed by drug inhibition assays (FIG. 8C). Pretreatment of cells with nystatin or methyl-β-cyclodextrin (MβCD), either of which is known to disrupt caveolin-dependent internalization, significantly decreased the uptake of CML particles in HeLa cells. However, no inhibitory effect on their uptake was observed when cells were pretreated with chlorpromazine (CPZ), a drug known to inhibit clathrin-dependent internalization by blocking clathrin polymerization. Results from the inhibition assay further verified that the entry of CMLs is mediated by caveolin-dependent endocytosis. It also appeared that incubation of CMLs with cells at 4° C. for 1 h significantly diminished the cellular uptake of CMLs by ˜66.7% compared to their internalization upon incubation at 37° C. for 1 h, verifying that CMLs enter cells via energy-dependent endocytosis.

Although caveolin-mediated entry and the subsequent intracellular processing remain poorly understood, cargos endocytosed through caveolae are believed to be transported to an organelle called “caveosome”. Cargo that traffics through the caveosome is thought to be further transported to the Golgi apparatus and/or endoplasmic reticulum (ER). It is also proposed that caveosomes may fuse directly to the early endosomes in a GTPase Rb5-dependent manner and may also proceed through the conventional endocytic pathway (endosomes/lysosomes). To further demonstrate the subsequent intracellular fate of CMLs, DiD-labeled CML particles were evaluated for their colocalization with the early endosome (EEA-1), lysosome (Lamp-1), and trans-Golgi (TGN-38) markers at different incubation times at 37° C. After incubation of 45 min, most CML particles were found in the EEA1⁺ early endosomes, whereas much less colocalization was detected between CMLs and EEA1 after 2 h incubation. Rather, at 2 h incubation, CML particles were mainly found in lysosomes, and some colocalization of CMLs with trans-Golgi was also observed. These imaging results demonstrated that CML particles could be primarily trafficked from caveosomes to the early endosome-lysosome compartments and could also traffic to the trans-Golgi network, possibly through the early endosomes.

In addition, imaging of drug release with Dox-loaded CMLs showed that a large amount of dot-shaped Dox fluorescence could be observed in the cytoplasm after 45 min incubation in HeLa cells, which suggests that Dox-liposome complexes were still located in the endosomes without significant release of Dox. After 2 h incubation, Dox fluorescent signals had diffused out of liposomes into the cytoplasm, but some dot-shaped Dox fluorescence could still be observed. At 3 h incubation, Dox fluorescence was mainly detected in the nucleus of cells with no clear observation of dot-shaped Dox in the cytoplasm, indicating that CML-Dox particles were most likely transported up to lysosomes where the encapsulated Dox was released into the cytoplasm prior to lysosomal degradation.

In Vivo Toxicity, Cardiac Toxicity, and Tolerability.

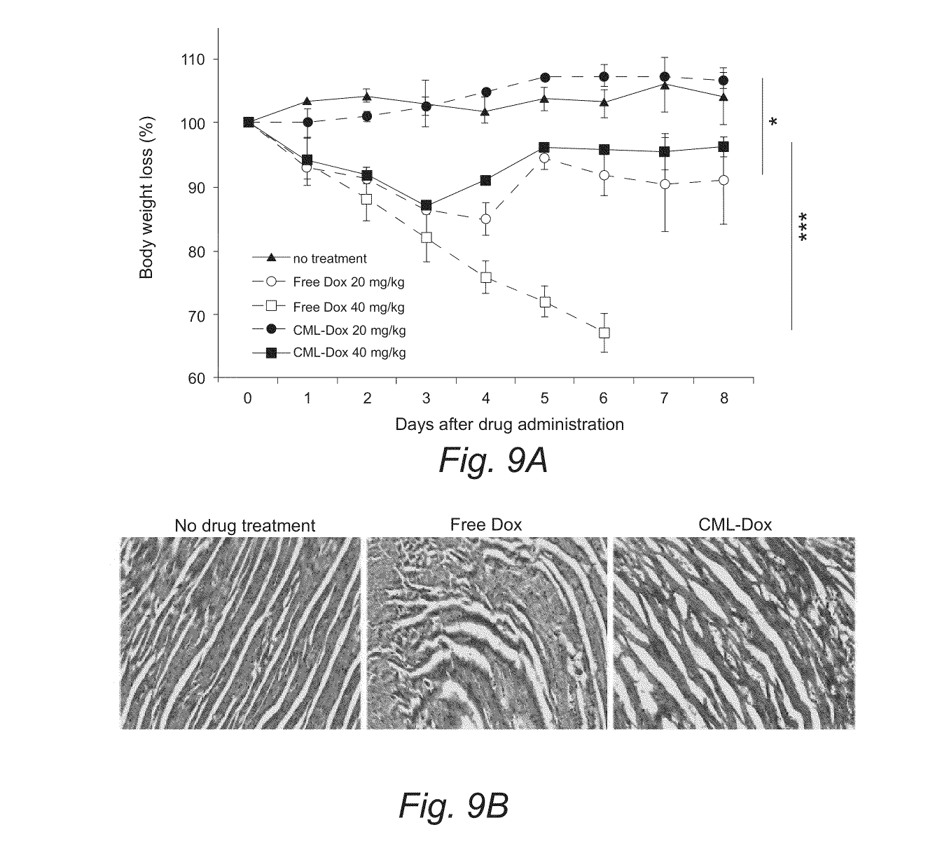

Despite recent advances in chemotherapeutic agents for cancer, their clinical applications are often limited by systemic toxicity. Therefore, various formulations of the drugs have been evaluated to achieve reduction in systemic toxicity. To determine the toxicity and tolerability of CML-Dox, we estimated the maximum tolerated dose by a single intravenous administration to C57BL/6 mice. The weights and general health of the mice were monitored for 8 days after injection of CML-Dox or free Dox at doses of 0, 20 and 40 mg/kg Dox equivalents (FIG. 9A). As expected, a significant loss of body weight was observed at both 20 and 40 mg/kg of free Dox. Especially, mice receiving 40 mg/kg of free Dox exhibited obvious signs of toxicity. However, mice in the groups receiving CML-Dox appeared healthy. Mice receiving 20 mg/kg of CML-Dox showed no loss of weight throughout the experiment. Some loss of weight was observed in mice receiving 40 mg/kg of CML-Dox, but body weights were recovered 4 days post-injection. The results indicated that CML-Dox was much less toxic to mice (maximum tolerated dose >40 mg/kg) than free Dox (maximum tolerated dose <20 mg/kg). Furthermore, histopathologic analysis indicated that free Dox (20 mg/kg) caused severe damage of cardiac tissue such as myofibrillary loss and disarray, whereas no significant histopathologic changes in cardiac tissue from mice treated with CML-Dox (20 mg/kg Dox equivalent) or no drug (FIG. 9B).

In Vivo Therapeutic Antitumor Efficacy.

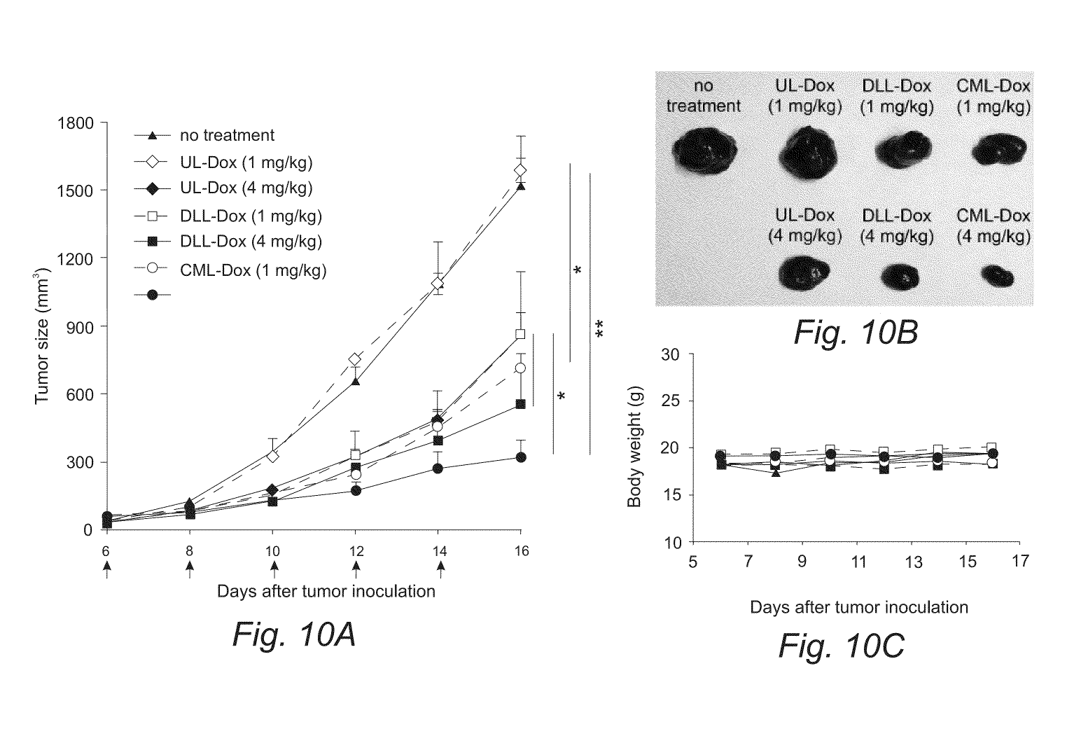

Next, a mouse tumor model was used to validate the therapeutic efficacy of the CML-Dox formulation, compared with that of UL-Dox or DLL-Dox. At day 0, C57/BL6 mice were inoculated subcutaneously with B16 melanoma tumor cells. On day 6, mice were injected intravenously with UL-Dox, DLL-Dox, or CML-Dox at doses of 1 or 4 mg/kg Dox equivalents every other day, and tumor growth and body weights were then monitored for an additional 10 days. Mice in the group receiving 1 mg/kg CML-Dox showed significant tumor inhibition, whereas the treatment of mice with the equivalent Dox concentration of UL-Dox exhibited no inhibition at all (FIG. 10A). At the higher dose of CML-Dox (4 mg/kg Dox), a dramatic suppression of tumor growth was observed in the group (FIGS. 10A and 10B), representing a significantly augmented therapeutic efficacy compared to that of UL-Dox. It also showed that CML-Dox exhibited slightly better antitumor effect compared with the conventional liposome formulation, DLL-Dox. No weight loss was seen for the duration of the experiment, even at the high dose of 4 mg/kg (FIG. 10C), indicating the absence of systemic toxicity from this CML formulation.

Positron Emission Tomography (PET) Imaging, Pharmacokinetics, and Biodistribution.

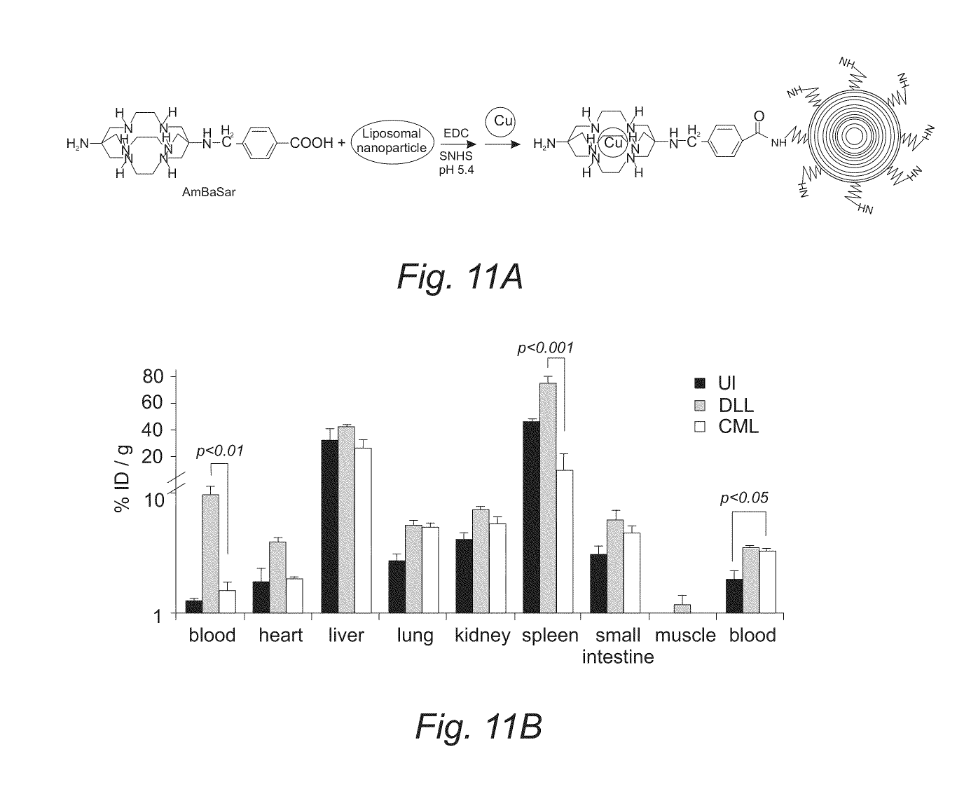

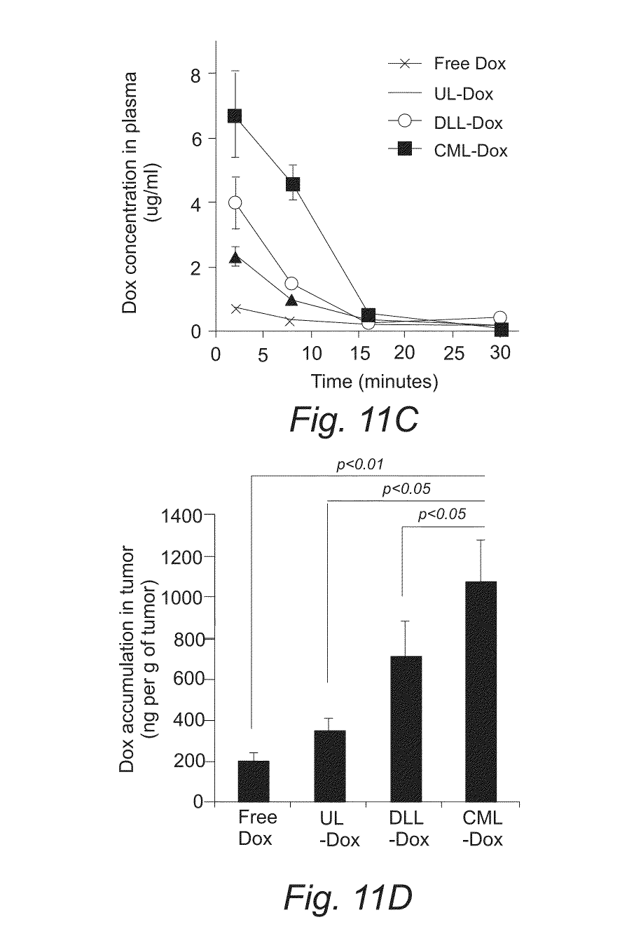

To further investigate the basis of the enhanced therapeutic effectiveness of CML-Dox compared to that of UL-Dox or DLL-Dox, biodistribution patterns of liposomes were evaluated in mice bearing B16 tumors with in vivo PET imaging. The process for preparing radiolabeled liposomes is shown in FIG. 11A. The bifunctional chelator AmBaSar was used in the ⁶⁴Cu labeling due to the superior in vivo stability of ⁶⁴Cu-AmBaSar over other ⁶⁴Cu-chelator, such as ⁶⁴Cu-DOTA (1,4,7,10-tetraazacyclododecane-1,4,7,10-tetraacetic acid). The PET images were obtained at several time points (1, 3, 24 h) after intravenous injection of ⁶⁴Cu-AmBaSar-labeled ULs, DLLs, or CMLs. After 1 h of administration, radioactivity was present mainly in well-perfused organs, and accumulation in tumors was detected in DLLs and CMLs compared with ULs. Furthermore, the accumulation of DLLs and CMLs in tumors significantly increased after 3 and 24 h of injection, whereas accumulation of ULs in the bladder was observed after 3 h of administration as a consequence of rapid degradation.

In addition, the tumors and tissues of interest were then excised at 24 h post-injection and weighed, and accumulation levels of particles in the tumors and tissues were determined by measuring radioactivity (FIG. 11B). This biodistribution assay revealed significantly higher accumulation of CMLs in tumors than that of ULs with the same lipid composition, suggesting that CMLs with improved vesicle stability could indeed enhance accumulation of drug carriers at the tumor site. Although no significant difference was observed between CMLs and DLLs relative to the accumulation of liposomes in the tumors, it is noteworthy that remarkably higher accumulation of DLLs in blood, heart, and spleen was detected compared to that of CMLs. This result indicated that DLLs could exhibit longer blood circulation compared to that of CMLs, but it could also lead to unwanted drug distribution to heart and spleen, which could induce severe toxic side effects, including cardiac toxicity.

The pharmacokinetic analysis showed that Dox levels of CML-Dox in serum were higher than those of free Dox and UL-Dox and, interestingly, even higher than Dox levels of DLL-Dox in serum at early time points (within 15 min) after injection (FIG. 11C). This observation may suggest that DLL particles were preferentially absorbed by tissues, such as spleen and heart, quickly after administration. However, after having accumulated in tissues, some particles were slowly returned to blood circulation, thus explaining the longer blood retention of DLLs compared to that of CMLs at later times. In addition, Dox levels accumulated into tumors were further examined by measuring Dox concentration in collected tumors at 24 h after administration. As shown in FIG. 11D, a significantly higher Dox level was detected in the tumors treated with CML-Dox, compared to that of free Dox, UL-Dox, or DLL-Dox. This result confirmed that the CML formulation could enhance penetration of Dox into the tumors. Moreover, even though both CMLs and DLLs exhibited a similar level of liposome accumulation in tumors, CMLs could achieve a higher Dox concentration in tumors based on their improved drug release (i.e., enhanced drug bioavailability), which corresponded well with the improved antitumor activity of CML-Dox in the mouse tumor model.

### Discussion

The overarching aim of this study was to evaluate a crosslinked multilamellar liposomal formulation of the anticancer agent doxorubicin for cancer therapeutics. We have demonstrated that crosslinked multilamellar structures of the CMLs not only offer controllable and sustainable drug release kinetics with increased vesicle stability, but also provide enhanced drug bioavailability, compared to the conventional unilamellar liposomes. It was also demonstrated that CMLs stably entrapped Dox in the vesicle and that the remarkable stability of CMLs allowed for long-term storage without a significant change in their size properties. Although some studies reported toxicity of some thiol-containing compounds including DTT in various cell lines, which is mainly caused by thiol-induced apoptosis, all thiol groups of DTT used as the cross-linker during the CML synthesis are quantitatively reacted with the maleimide headgroup of the lipid, and additionally, unreacted DTT was removed from CMLs after crosslinking. Thus, the thiol-induced toxicity should be minimized in this CML formulation.

In the present study, we demonstrated that the enhanced delivery of CML-Dox to tumor cells in vitro and in vivo improved anticancer activity and led to better tumor reduction and inhibition of tumor progression, when compared with the antitumor activity of non-crosslinked unilamellar liposome with the same lipid composition (UL) or Doxil-like liposome (DLL). Generally, the vesicle stability and drug release rate of liposomes are determined by liposome size, structure, and lipid composition. Although a higher drug release rate could augment drug bioavailability in tumors, the rapid release of drug from liposomes usually causes an equally rapid drug clearance in plasma, resulting in lower therapeutic efficacy to tumors. However, the crosslinked multilamellar structure of CML could achieve a controlled and sustained drug release profile, even though CML was composed of low-Tₘ (transition temperature) phospholipids, thus resulting in enhanced and sustained drug release kinetics. Consequently, the increased bioavailability of CML-Dox, together with improved vesicle stability, could allow for higher therapeutic activity, both in vitro and in vivo.

Furthermore, the entry mechanism and subsequent intracellular trafficking of CMLs were determined by direct visualization of interactions between CMLs and cellular endocytic structures. Our imaging study suggested that CML particles enter cells via caveolin-dependent endocytosis. Several studies have demonstrated that the caveolin-mediated pathway is a main route for uptake of liposomes. Moreover, efforts have been made in recent years to elucidate the detailed molecular mechanism underlying caveolae-mediated endocytosis and the subsequent intracellular fate of cargos. For example, simian virus 40 (SV40) is known to utilize the caveolae-mediated pathway for its entry and to be transported from the caveosome to endoplasmic reticulum (ER) to mediate infection. Cholera toxin binding subunit (CTxB) is also believed to enter cells in a caveolin-dependent manner and to traffic to the Golgi complex, possibly through early endosomes. Another recent study demonstrated that caveolin-dependent infectious entry of human papillomavirus type 31 (HPV31) could proceed to the endolysosomal compartments for successful infection. These prior studies indicate that cargos utilizing caveolin-mediated endocytosis can traffic to multiple distinct intracellular destinations. Similarly, our imaging results with intracellular organelles suggested that CML particles internalized through caveolae might be predominantly transported further through the conventional endocytic pathway (i.e., early endosome to lysosome) and also traffic from the early endosomes to the trans-Golgi network, indicating that intracellular trafficking of CMLs might involve multiple distinct intracellular pathways. Additionally, it also appeared that the encapsulated Dox was released to cytoplasm of the cell prior to lysosomal degradation. The process for releasing the drug from the liposomes presumably involves the disruption of the integrity of the liposome bilayer in the presence of phospholipases, i.e., enzymes that hydrolyze phospholipids into fatty acids and other lipophilic substances present within endolysosomal compartments.

Cholesterol is well known to rigidify and stabilize the liposomal membranes and has been widely used for current liposomal formulations. Addition of cholesterol to CML formulation may also provide rigid bilayers, promoting drug retention. However, incorporation of cholesterol in lipid bilayers could also hamper the process of crosslinking inter-lipid bilayers, which leads to vesicle instability in CML formulation. In addition, it was previously reported that the presence of cholesterol in the liposome membrane dramatically inhibits phospholipase activity, which suggests that cholesterol might disrupt cellular drug release from endolysosomal compartments and then decrease cytotoxic activity in tumor cells.

A previous report showed that the interbilayer-crosslinked multilamellar vesicle carrying protein antigens and adjuvants could steadily generate potent humoral and cellular immune responses for vaccine delivery. In our study, we further extended this liposomal formulation to anticancer therapeutics and demonstrated many promising features of CMLs as a new nanocarrier platform for chemotherapy drug delivery applications. The CML formulation of Dox could reduce systemic toxicity, most likely by the controlled drug release. Furthermore, enhanced vesicle stability with higher Dox bioavailability enabled the improved in vivo therapeutic activity to tumors. It is noteworthy that CML-Dox treatment of B16 tumors, which is known as one of the most aggressive types of tumors, exhibited significant inhibition of tumor growth compared to that treated with the conventional liposomes UL-Dox and DLL-Dox. Our biodistribution study revealed that the enhanced therapeutic efficacy of CMLs resulted from the augmented accumulation of drugs at tumor sites and also showed lower accumulation of CMLs in heart and spleen compared to that of DLLs, which could improve the effectiveness and safety of drugs by minimizing the unwanted side effects.

2. Enhanced Therapeutic Efficacy of iRGD-Conjugated Crosslinked Multilayer Liposomes for Drug Delivery

### Materials and Methods

### Materials

Mice.

Female 6- to 10-week-old BALB/c mice were purchased from Charles River Breeding Laboratories (Wilmington, Mass.). All mice were held under specific pathogen-reduced conditions in the Animal Facility of the University of Southern California (USA). All experiments were performed in accordance with the guidelines set by the National Institutes of Health and the University of Southern California on the Care and Use of Animals.

Cell Lines, Antibodies, and Reagents.

4T1 tumor cells (ATCC number: CRL-2539) and JC cells (ATCC number: CRL-2116) were maintained in a 5% CO₂ environment with Dulbecco's modified Eagle's medium (Mediatech, Inc., Manassas, Va.) supplemented with 10% FBS (Sigma-Aldrich, St. Louis, Mo.) and 2 mM of L-glutamine (Hyclone Laboratories, Inc., Omaha, Nebr.). The mouse monoclonal antibodies against clathrin, caveolin-1 and EEA1 were purchased from Santa Cruz Biotechnology, Inc. (Santa Cruz, Calif.). The mouse monoclonal antibody to Lamp-1 was purchased from Abcam (Cambridge, Mass.). Alexa488-TFP ester and Alexa488-goat anti-mouse immunoglobulin G (IgG) were obtained from Invitrogen (Carlsbad, Calif.). Chlorpromazine (CPZ) and Filipin were obtained from Sigma-Aldrich (St. Louis, Mo.) and used at appropriate concentrations according to the manufacturer's protocols.

Synthesis of iRGD-cMLVs.

Preparation of liposomes was based on the conventional dehydration-rehydration method. All lipids were obtained from NOF Corporation (Japan). 1.5 μmol of lipids 1,2-dioleoyl-sn-glycero-3-phosphocholine (DOPC), 1,2-dioleoyl-sn-glycero-3-phospho-(1′-rac-glycerol) (DOPG), and maleimide-headgroup lipid 1,2-dioleoyl-sn-glycero-3-phosphoeth-anolamine-N-[4-(p-maleimidophenyl) butyramide (MPB-PE) were mixed in chloroform to form a lipid composition with a molar ratio of DOPC:DOPG:MPB=4:1:5, and the organic solvent in the lipid mixture was evaporated under argon gas, followed by additional drying under vacuum overnight to form dried thin lipid films. The resultant dried film was hydrated in 10 mM Bis-Tris propane at pH 7.0 with doxorubicin at a molar ratio of 0.2:1 (drugs:lipids) with vigorous vortexing every 10 min for 1 h and then applied with 4 cycles of 15-s sonication (Misonix Microson XL2000, Farmingdale, N.Y.) on ice at 1 min intervals for each cycle. To induce divalent-triggered vesicle fusion, MgCl₂ was added to make a final concentration of 10 mM. The resulting multilamellar vesicles were further crosslinked by addition of dithiothreitol (DTT, Sigma-Aldrich) at a final concentration of 1.5 mM for 1 h at 37° C. The resulting vesicles were collected by centrifugation at 14,000 g for 4 min and then washed twice with PBS. For iRGD conjugation to cMLVs, the particles were incubated with 0.5 μmol of iRGD peptides (GenScript, Piscataway, N.J.) for 1 h at 37° C. For pegylation of cMLVs, both unconjugated and iRGD-conjugated particles were further incubated with 0.5 μmol of 2 kDa PEG-SH (Laysan Bio Inc., Arab, Ala.) for 1 h at 37° C. The particles were then centrifuged and washed twice with PBS. The final products were stored in PBS at 4° C.

Characterization of Physical Properties.

The hydrodynamic size and size distribution of iRGD-cMLVs were measured by dynamic light scattering (Wyatt Technology, Santa Barbara, Calif.).

In Vitro Drug Encapsulation and Release.

To study the loading capacity of Dox, iRGD-cMLV(Dox) nanoparticles were collected and then washed twice with PBS, followed by lipid extraction of vesicles with 1% Triton X-100 treatment. Dox fluorescence (excitation 480 nm, emission 590 nm) was then measured by a Shimadzu RF-5301PC spectrofluorometer (Japan). To obtain the release kinetics of Dox from liposomes, Dox-loaded iRGD-cMLVs were incubated at 37° C. in 10% fetal bovine serum (FBS)-containing media, the releasing media were removed from iRGD-cMLVs incubated at 37° C. for quantification of Dox fluorescence every day, and fresh media were replaced for continuous monitoring of drug release.

In Vitro Cytotoxicity.

4T1 and JC cells were plated at a density of 5×10³ cells per well in D10 media in 96-well plates and grown for 6 h. The cells were then exposed to a series of concentrations of cMLV(Dox) or iRGD-cMLV(Dox) for 48 h, and the cell viability was assessed using the Cell Proliferation Kit II (XTT assay) from Roche Applied Science (Indianapolis, Ind.) according to the manufacturer's instructions. Cell viability percentage was determined by subtracting absorbance values obtained from media-only wells from drug-treated wells and then normalizing to the control cells without drugs. The data were analyzed by nonlinear regression to get the IC₅₀ value.

In Vitro Binding and Internalization Study.

4T1 cells were plated at a density of 2×10⁵ cells per well in D10 media in 24-well plates and grown overnight. The cells were incubated with two concentrations (0.2 μg/ml and 0.04 μg/ml) of iRGD-cMLV(Dox) or cMLV(Dox) for 30 min at 4° C. (for binding assay) or 2 h at 37° C. (for internalization assay). After incubation, the cells were washed twice with PBS to remove the unbound nanoparticles. Binding and cellular uptake of particles were determined by measuring doxorubicin fluorescence using flow cytometry.

Confocal Imaging.

Fluorescence images were acquired on a Yokogawa spinning-disk confocal scanner system (Solamere Technology Group, Salt Lake City, Utah) using a Nikon eclipse Ti-E microscope equipped with a 60×/1.49 Apo TIRF oil objective and a Cascade II: 512 EMCCD camera (Photometrics, Tucson, Ariz., USA). An AOTF (acousto-optical tunable filter) controlled laser-merge system (Solamere Technology Group Inc.) was used to provide illumination power at each of the following laser lines: 491 nm, 561 nm, and 640 nm solid state lasers (50 mW for each laser).

To label liposomal particles, DiD lipophilic dyes were added to the lipid mixture in chloroform at a ratio of 0.01:1 (DiD:lipids), and the organic solvent in the lipid mixture was evaporated under argon gas to incorporate DiD dyes into a lipid bilayer of vesicles. To detect iRGD peptides, both iRGD-cMLV and unconjugated cMLV particles were incubated with 50 nmol of Alexa488-TFP ester (Invitrogen) for 2 h in 0.1 M sodium bicarbonate buffer (pH=9.3). After 2 h incubation, the reaction was stopped, and unbound dye molecules were removed via buffer exchange into PBS (pH=7.4) using a Zeba desalting spin column (Fisher Scientific). For the detection of intracellular nanoparticles, DiD-labeled iRGD-cMLV or DiD-labeled unconjugated cMLV were incubated for 30 min at 4° C. with HeLa cells that were seeded overnight on polylysine-coated glass bottom dishes (MatTek Corporation, Ashland, Mass.). Then the samples were incubated at 37° C. to initiate particle internalization at the indicated time points. The culture dish was then rinsed, fixed with 4% formaldehyde, permeabilized with 0.1% Triton X-100, and then immunostained with the corresponding antibodies specific to clathrin, caveolin-1, EEA1, or Lamp-1 and counterstained with DAPI (Invitrogen, Carlsbad, Calif.).

Uptake Inhibition Assay.

HeLa cells (1×10⁵ cells) were preincubated with chlorpromazine (CPZ, 25 μg/ml) or filipin (10 μg/ml) for 30 min to disrupt the clathrin- or caveolin-mediated pathway. The cells were then incubated with DiD-labeled iRGD-cMLV or unconjugated cMLV for 1 h at 37° C. in the presence of CPZ and filipin. The cells were then washed twice with PBS. The cellular uptake of particles was determined by measuring DiD fluorescence using flow cytometry and normalized on the basis of fluorescent intensity acquired from the untreated cells.

In Vivo Antitumor Activity Study.

BALB/c female mice (6-10 weeks old) were inoculated subcutaneously with 0.2×10⁶ 4T1 breast tumor cells. The tumors were allowed to grow to a volume of ˜50 mm³ before treatment. On day 10, the mice were injected intravenously through tail vein with PBS (control group), cMLV (2 mg/kg Dox) and iRGD-cMLV (2 mg/kg Dox) every three days (five mice per group). Tumor growth and body weight were then monitored until the end of the experiment. The length and width of the tumor masses were measured with a fine caliper every three days after injection. Tumor volume was expressed as ½×(length×width²).

### Results

Preparation of iRGD-cMLV Nanoparticles.

The procedure for the preparation of crosslinked multilayer liposomal vesicles (cMLV) was adapted from a recently reported multistep procedure based on the conventional dehydration-rehydration method to form covalent crosslinkers between adjacent lipid bilayers (Moon et al., 2011), as illustrated in FIGS. 2A and 2B. This method employed a divalent cation-triggered vesicle fusion to yield a multilamellar structure, from which inter-bilayer crosslinkers were formed across the opposing sides of lipid bilayers through the reactive headgroups with dithiothreitol (DTT). The iRGD peptides (CRGDKGPDC) were conjugated to the surface of cMLVs through the functional thiol-reactive maleimide headgroups of maleimide-headgroup lipid, 1,2-dioleoyl-sn-glycero-3-phosphoeth-anolamine-N-[4-(p-maleimidophenyl) butyramide (MPB-PE). As a final step, the surface of the iRGD-conjugated cMLV (iRGD-cMLV) was pegylated with thiol-terminated PEG to further improve the blood circulation time of vesicles.

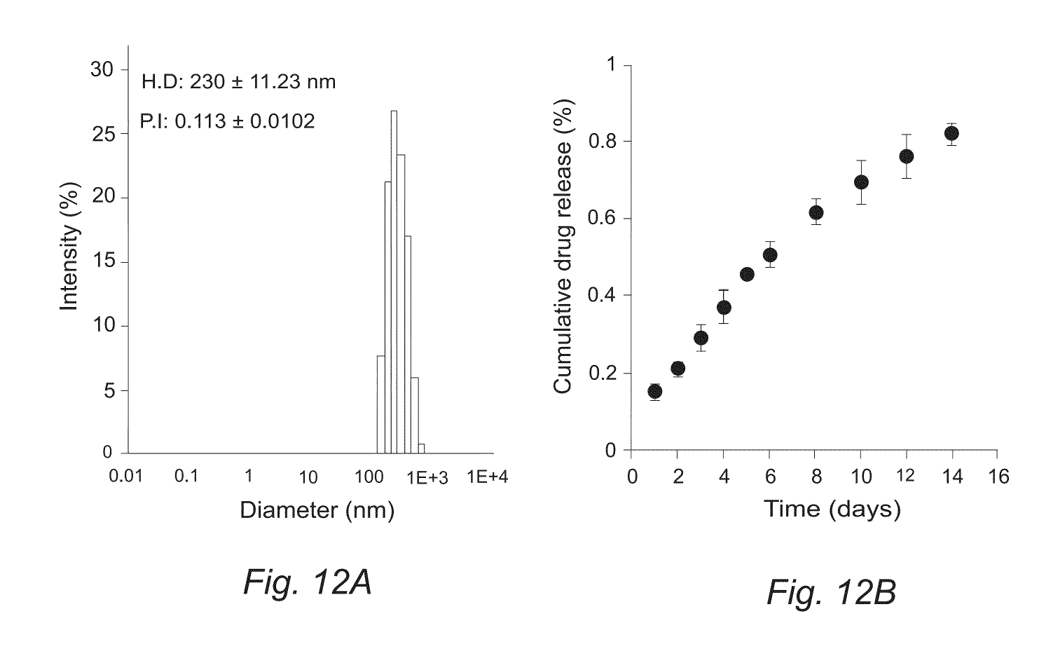

The physical properties of synthesized iRGD-cMLV were characterized. The hydrodynamic size of these targeted nanoparticles was measured by dynamic light scattering (DLS), and the result showed the mean diameter of iRGD-cMLV to be ˜230±11.23 nm (FIG. 12A), which was similar to that of unconjugated cMLV (˜220±6.98 nm). Moreover, it has been confirmed that doxorubicin (Dox)-encapsulation efficiency of ˜85% can be achieved via this preparation procedure. An in vitro drug release assay also showed that iRGD-cMLV exhibited slow and sustained release kinetics (up to 2 weeks) in a serum environment (FIG. 12B).

Next, we examined whether iRGD peptides were conjugated to the surface of cMLV via the maleimide headgroups. To this end, fluorescent 1,1-dioctadecyl-3,3,3,3-tetramethylindodicarbocyanine (DiD)-labeled cMLV particles were used to visualize both unconjugated and conjugated particles. In addition, Alexa488 dye was utilized to label iRGD peptides through the amine group of lysine residues on iRGD peptides (CRGDKGPDC). The results showed that a significant colocalization of DiD-labeled iRGD-cMLV particles with Alexa488-labeled iRGD peptides was observed, while no Alexa488 signals were detected on unconjugated cMLV particles, suggesting that iRGD peptides were successfully conjugated to cMLV particles.

Cytotoxicity and Cell Uptake of iRGD-cMLV(Dox).

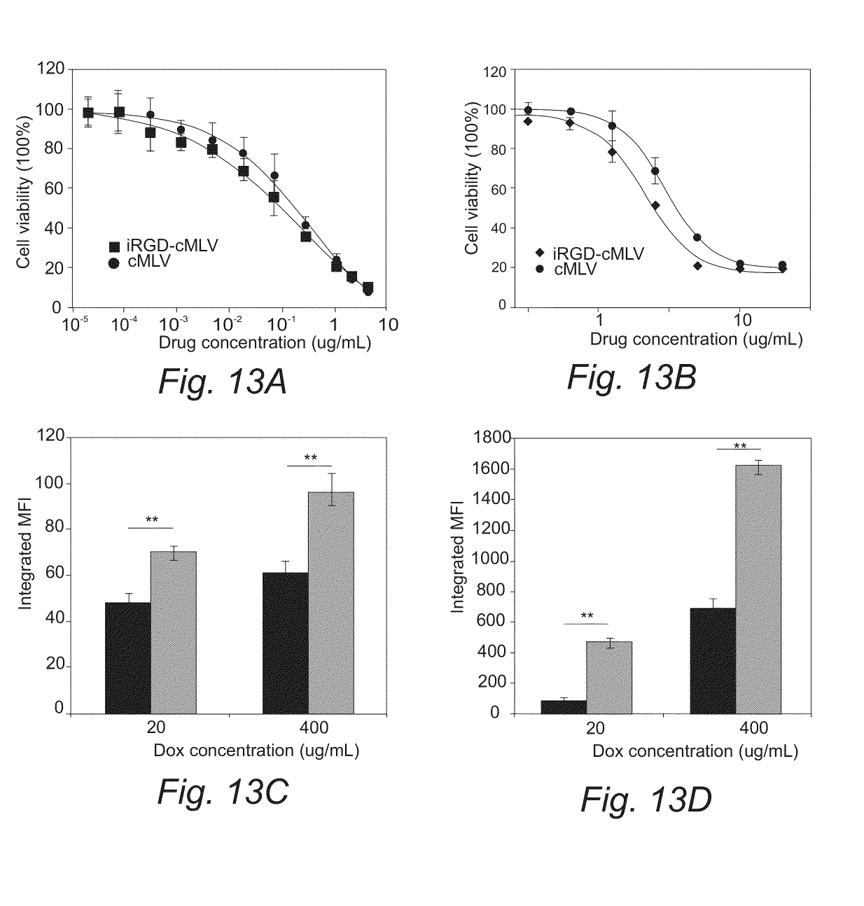

We next determined the effect of iRGD-conjugated cMLV nanoparticles on cytotoxicity levels in cells as compared to unconjugated cMLV nanoparticles. Dox-loaded cMLV (cMLV(Dox)) and Dox-loaded iRGD-cMLV (iRGD-cMLV(Dox)) were incubated with 4T1 or JC cells. JC cells represent a model drug-resistant tumor cell line overexpressing P-glycoprotein and exhibiting drug-resistant phenotype both in vitro and in vivo. After 48 h incubation, the cytotoxicity of Dox-liposomes was measured by a standard XTT assay. In vitro cytotoxicity data revealed that iRGD-cMLV showed slightly smaller IC₅₀ (0.011±0.0037 μg/ml) in 4T1 cells as compared to cMLV (0.018±0.0025 μg/ml) (FIG. 13A). A significant difference of cytotoxicity between iRGD-cMLV(Dox) and cMLV(Dox) was observed in JC cells, in which iRGD-cMLV(Dox) showed a lower IC₅₀ (2.01±0.22 μg/ml) value than that of cMLV(Dox) (3.19±0.32 μg/ml, P<0.05, FIG. 13B). The XTT results indicated that delivery of Dox with iRGD-conjugated cMLV was more potent in inhibiting tumor cell proliferation.

To investigate whether the enhanced cell cytotoxicity of iRGD-cMLV resulted from an increased cellular uptake of nanoparticles, the cellular binding and uptake of iRGD-cMLV and cMLV were examined. For the binding assay, cMLV(Dox) or iRGD-cMLV(Dox) were incubated with 4T1 tumor cells at 4° C. for 30 min. Then the bound nanoparticles on the cell surface were determined by detecting doxorubicin signals via flow cytometry after removing the unbound nanoparticles. As shown in FIG. 13C, at both concentrations, a significantly higher integrated mean fluorescence intensity (MFI) was observed when the cells were incubated with iRGD-cMLV(Dox), indicating that iRGD-cMLVs can facilitate the attachment of nanoparticles to the cells via the integrin receptor expressed on the surface of tumor cells (P<0.01). Additionally, the cellular accumulation of doxorubicin in 4T1 cells was determined by integrated MFI after the cells were incubated with cMLV(Dox) or iRGD-cMLV(Dox) at 37° C. for 2 h. The results showed that a remarkably enhanced cell uptake of doxorubicin was observed when the cells were incubated with iRGD-cMLV(Dox) (P<0.01, FIG. 13D), suggesting that the increased cellular accumulation of doxorubicin was facilitated by iRGD peptides. Taken together, the iRGD peptides promoted both binding and uptake of drug-loaded nanoparticles in tumor cells, thereby enhancing the drug concentration in cells and improving the cytotoxicity of drugs.

Internalization and Intracellular Pathways of iRGD-cMLVs.

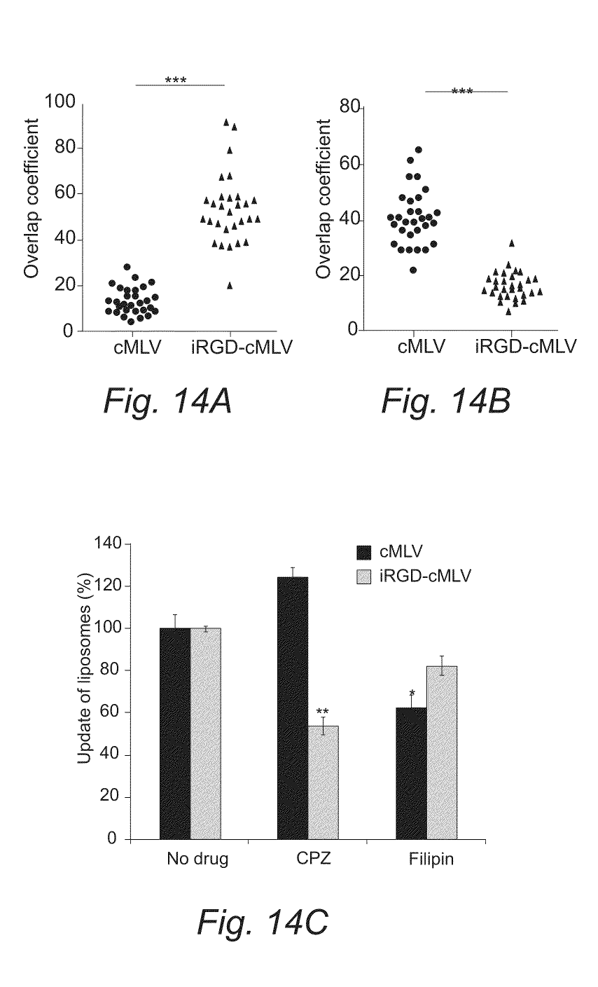

We next investigated the entry mechanism and intracellular process of iRGD-cMLV into tumor cells to determine whether iRGD peptides could change the pathway by which nanoparticles are endocytosed. Endocytosis is known as one of the main entry mechanisms for various nanoscale drug carriers. Several studies have reported the involvement of clathrin- and caveolin-dependent pathways in nanoparticle-mediated endocytosis. Therefore, to investigate the role of clathrin- or caveolin-dependent endocytosis of iRGD-cMLVs, we visualized the individual fluorescent DiD-labeled cMLVs or iRGD-cMLVs with endocytic structures (clathrin or caveolin) after 15 min incubation at 37° C. A significant colocalization of unconjugated cMLV particles with caveolin-1 signals was observed, while no colocalization between unconjugated cMLV particles and clathrin structures was detected, indicating that the caveolin pathway may be involved in the endocytosis of cMLVs. However, after 15 min incubation, iRGD-cMLV particles were colocalized with clathrin structures, whereas, no significant colocalization between iRGD-cMLV particles and caveolin-1 signals was observed, suggesting that the endocytosis of iRGD-cMLVs could be clathrin-dependent. The quantification of iRGD-cMLVs and cMLVs colocalized with caveolin-1 or clathrin structures by analyzing more than 30 cells confirmed that the clathrin-mediated pathway could be involved in the entry of iRGD-cMLVs, while the endocytosis of cMLVs could be caveolin-1-dependent (FIGS. 14A and 14B). The role of clathrin-dependent endocytosis of iRGD-cMLV was further examined by drug-inhibition assays shown in FIG. 14C. Chlorpromazine (CPZ) is known to block clathrin-mediated internalization by inhibiting clathrin polymerization, while filipin is a cholesterol-binding reagent that can disrupt caveolin-dependent internalization. As shown in FIG. 14D, CPZ (10 μg/ml) significantly decreased the uptake of iRGD-cMLV particles in HeLa cells, while no significant inhibitory effect on their uptake was observed when cells were pretreated with Filipin (10 μg/ml). However, pretreatment of cells with Filipin remarkably decreased the uptake of unconjugated cMLV particles (P<0.01), whereas no inhibitory effect on their uptake was observed in CPZ-pretreated cells. Results from the inhibition assay further confirmed that iRGD-cMLV endocytosis is mediated by the clathrin-dependent pathway, while unconjugated cMLV particles enter cells via caveolin-dependent endocytosis.

Once inside the cells, the intracellular fate of the endosomal contents has been considered as an important determinant of successful drug delivery. It was also proposed that nanoparticles might transport to the early endosomes in a GTPase Rb5-dependent manner and also proceed through the conventional endocytic pathway (endosomes/lysosomes), probably resulting in enzymatic destruction of lipid membrane for drug release in lysosomes. To further investigate the subsequent intracellular fate of iRGD-cMLV nanoparticles, DiD-labeled iRGD-cMLV particles were evaluated for their colocalization with the early endosome (EEA-1) and lysosome (Lamp-1) markers at different incubation times at 37° C. Most iRGD-cMLV particles were found in the EEA1⁺ early endosomes after incubation of 30 min, validating the involvement of early endosomes in the intracellular fate of targeted cMLV particles. In addition, after 2 h incubation, a significant colocalization of iRGD-cMLVs with lysosomes was observed, suggesting that iRGD-cMLVs may transport to early endosomes and further travel to lysosomes for possible release of drug from liposomes and endocytic compartments to cytosol. When taken together, the results showed that iRGD-cMLVs enter tumor cells via clathrin-dependent and receptor-mediated endocytosis, followed by transport through early endosomes and lysosomes.

Therapeutic Effect of iRGD-cMLV(Dox) in Breast Tumor Animal Model.

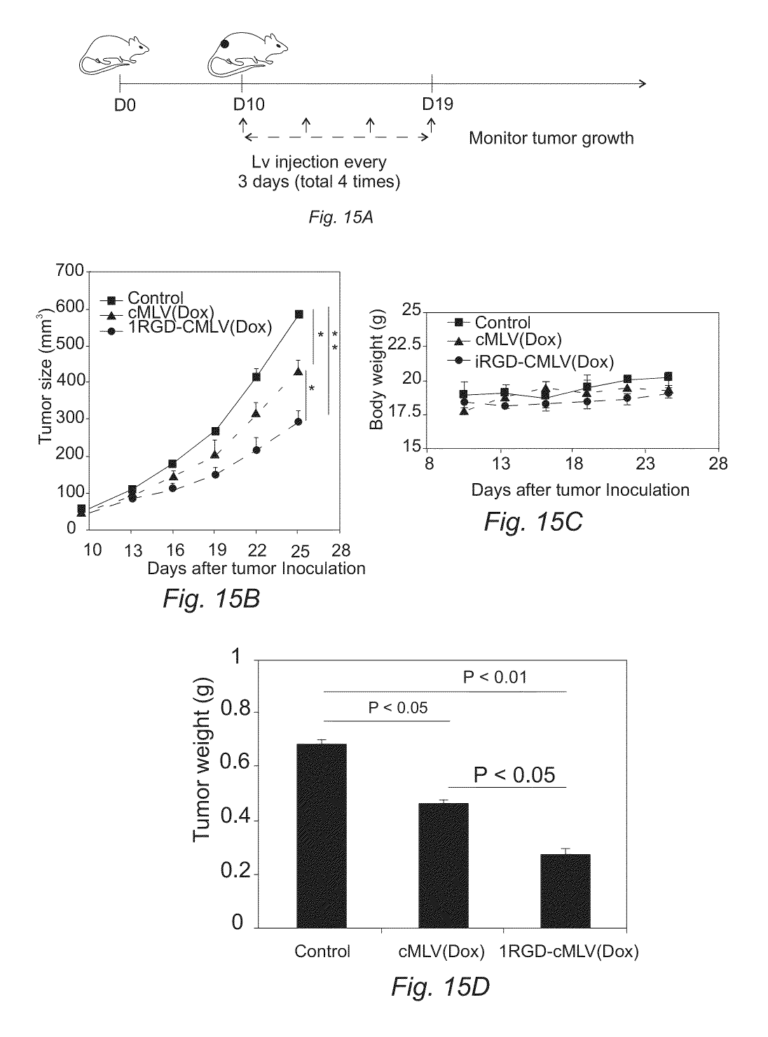

We have demonstrated that iRGD-conjugated cMLVs can enhance uptake of nanoparticles into cells, resulting in an increased concentration of doxorubicin and in vitro cytotoxicity. Here, a breast tumor animal model was used to evaluate the in vivo therapeutic efficacy of iRGD-cMLV(Dox), compared with that of cMLV(Dox). At day 0, BALB/c mice were inoculated subcutaneously with 4T1 breast tumor cells. At day 10, mice were injected intravenously with iRGD-cMLV(Dox) or cMLV(Dox) at doses of 2 mg/kg Dox equivalents every three days. Tumor growth and body weight were then monitored until the end of the experiment (FIG. 15A). As shown in FIG. 15B, mice in the group receiving 2 mg/kg cMLV(Dox) showed a significant tumor inhibition as compared to mice in the untreated group (P<0.01). In addition, a marked suppression of tumor growth was observed in the group treated by iRGD-cMLV(Dox), suggesting that iRGD peptides could further enhance the therapeutic effect of drug-loaded nanoparticles in vivo. During the whole experiment, no weight loss of all mice was seen (FIG. 15C), indicating the absence of systemic toxicity from cMLV and iRGD-cMLV formulations. The enhanced antitumor activity of iRGD-cMLV (Dox) was further confirmed by a significant reduction on tumor weight of mice treated with iRGD-cMLV(Dox), as compared to that treated with cMLV(Dox) (FIG. 15D).

### Discussion

Nontargeted, long-circulating liposomes, such as Doxil/Caelyx, have been extensively evaluated to deliver chemotherapeutic drugs to treat cancers via the enhanced permeability and retention mechanism. Although significant efforts have been made to enhance their therapeutic activity, the relatively inherent instability of conventional liposomes in the presence of serum component, resulting in rapid drug release profile, has been considered as an obstacle in their development for cancer treatment. In order to develop a liposomal formulation with sustainable release kinetics and improved stability, a cMLV formulation of Dox has been explored as a new nanocarrier platform with promising features of enhanced vesicle stability and reduced systemic toxicity, resulting in improved in vivo therapeutic efficiency. Although cMLVs have shown improved antitumor activity, direct delivery of these particles with targeting ligands could potentially further enhance efficacy and minimize toxicity.

Most currently investigated targeting strategies concentrate on directing nanoparticles to tumor cells by utilizing the specific receptor/ligand overexpressed on tumor cells. For instance, RGD (arginine-glycine-aspartate) peptides have been conjugated to drug-loaded nanoparticles to target integrin receptors, which are overexpressed on neovascular endothelial cells. Although the development of targeted payload for anticancer drug delivery has shown potential enhanced therapeutic effect, poor penetration of nanoparticles to tumor cells still thwarts clinical treatment of solid tumor. Therefore, a novel iRGD peptide has been recently identified and reported to increase vascular and tissue penetration in a tumor-specific and neuropilin-1 (NRP-1)-dependent manner. The C-terminal motif CendR of iRGD peptide has been identified as a mediator of cell and tissue penetration through the interaction with neuropilin-1 receptor, a cell-surface receptor that is involved in the regulation of vascular permeability. For example, it has been reported that the successful infection of many viruses required proteolytic cleavage of capsid proteins to expose the CendR motifs to neuropilin-1 receptor, which could trigger the endocytosis of viral particles into cells. Moreover, several studies have reported that peptides containing CendR motifs could bind to NRP-1 receptor and cause cellular internalization and vascular leakage, suggesting that iRGD peptides could have similar effects when covalently coupled to a drug delivery nanocarrier. Previously, we demonstrated the enhanced therapeutic ability of cMLV formulations with reduced systemic toxicity, as compared to that of umilamellar liposome or Doxil-like liposomes. Therefore, in this study, we conjugated iRGD peptides to this relatively stable cMLV particles and evaluated, both in vitro and in vivo, the effect of these targeted nanoparticles. A similar accumulative drug release profile was observed in iRGD-cMLV formulation as compared to cMLV formulations, due to a similar size distribution and lipid composition of these two formulations. The results showed that iRGD-cMLVs presented superior cytotoxicity resulting from the enhanced binding and uptake of targeted nanoparticles in cells. Moreover, enhanced uptake and penetration of Dox via iRGD-cMLV vesicles enabled the improved in vivo therapeutic activity in tumors. iRGD-cMLVs treatment of 4T1 tumors exhibited significant inhibition of tumor growth compared to that treated with cMLVs, further suggesting the potential application of iRGD for drug delivery via nanoparticles.

Furthermore, our imaging study on the entry mechanism of iRGD-cMLVs provided some edifying details about the intracellular fate of these particles. Specifically, the results showed that iRGD-cMLV particles enter cells via clathrin-dependent endocytosis, while the internalization of unconjugated cMLV particles is caveolin-mediated. The different endocytic pathways utilized by iRGD-cMLVs might result from the interaction of nanoparticles with cells via iRGD-integrin binding. The results also suggested that the receptor-mediated internalization possibly promoted cell attachment, resulting in an enhanced cellular uptake. Although it has been hypothesized that multiple pathways were involved in endosomal transport, our data showed that both iRGD-cMLVs and cMLVs home to early endosomes and further traffic to lysosomes. The involvement of lysosome in the intracellular trafficking routes of both iRGD-cMLVs and cMLVs might facilitate drug release kinetics because enzymes, such as phospholipases, in the endolysosomal compartments can promote disruption of liposomal bilayers.

### 3. Codelivery of Doxorubicin and Paclitaxel by Crosslinked Multilamellar Liposome Enables Synergistic Antitumor Activity

### Materials and Methods

Cell Lines, Antibodies, Reagents, and Mice.

B16-F10 (ATCC number: CRL-6475) and 4T1 tumor cells (ATCC number: CRL-2539) were maintained in a 5% CO₂ environment with Dulbecco's modified Eagle medium (Mediatech, Inc., Manassas, Va.) supplemented with 10% FBS (Sigma-Aldrich, St. Louis, Mo.) and 2 mM of L-glutamine (Hyclone Laboratories, Inc., Omaha, Nebr.). Mouse anti-β-Actin and rabbit antibody against phospho-specific protein p44/42 MAPK (Erk 1/2) were purchased from Cell Signaling Technology (Danvers, Mass.). Goat anti-Rabbit IR Dye®680RD and Goat anti-Mouse IR Dye®800CW were obtained from LI-COR BioSciences (Lincoln, Nebr.). Doxorubicin, Paclitaxel, Daunorubicin and Doxetaxel were purchased from Sigma-Aldrich (St. Louis, Mo.).

All lipids were obtained from NOF Corporation (Japan): 1,2-dioleoyl-sn-glycero-3-phosphocholine (DOPC), 1,2-dioleoyl-sn-glycero-3-phospho-(10-rac-glycerol) (DOPG), and 1,2-dioleoyl-sn-glycero-3-phosphoethanolamine-N-[4-(p-maleimidophenyl)butyramide (maleimide-headgroup lipid, MPB-PE).

Female 6-10 weeks-old BALB/c mice were purchased from Charles River Breeding Laboratories (Wilmington, Mass.). All mice were held under specific pathogen-reduced conditions in the Animal Facility of the University of Southern California (Los Angeles, Calif., USA). All experiments were performed in accordance with the guidelines set by the National Institute of Health and the University of Southern California on the Care and Use of Animals.

Synthesis of cMLVs.

Liposomes were prepared based on the conventional dehydration-rehydration method. All lipids were obtained from NOF Corporation (Japan). DOPC, DOPG and MPB-PE were combined in chloroform, at a molar lipid ratio of DOPC:DOPG:MPB=4:1:5, and the organic solvent in the lipid mixture was evaporated under argon gas. The lipid mixture was further dried under vacuum overnight to form dried thin lipid films. To prepare cMLV (Dox+PTX), paclitaxel in organic solvent was mixed with the lipid mixture before formation of the dried thin lipid films. The resultant dried film was hydrated in 10 mM Bis-Tris propane at pH 7.0 with doxorubicin by vigorous vortexing every 10 min for 1 h, and then applied with 4 cycles of 15-s sonication (Misonix Microson XL2000, Farmingdale, N.Y.) on ice in 1 min intervals for each cycle. To induce divalent-triggered vesicle fusion, MgCl₂ was added at a final concentration of 10 mM. The resulting multilamellar vesicles were further crosslinked by addition of Dithiothreitol (DTT, Sigma-Aldrich) at a final concentration of 1.5 mM for 1 h at 37° C. The resulting vesicles were collected by centrifugation at 14,000 g for 4 min and then washed twice with PBS. For pegylation of cMLVs, the particles were incubated with 1 μmol of 2 kDa PEG-SH (Laysan Bio Inc. Arab, Ala.) for 1 h at 37° C. The particles were then centrifuged and washed twice with PBS. The final products were stored in PBS at 4° C.

Characterization of Physical Properties.

The hydrodynamic size and size distribution of cMLVs were measured by dynamic light scattering (Wyatt Technology, Santa Barbara, Calif.).

In Vitro Drug Encapsulation and Release.

To study the loading capacity of Dox, cMLV(Dox) and cMLV(Dox+PTX) were collected and washed twice with PBS, followed by lipid extraction of vesicles with 1% Triton X-100 treatment. Dox fluorescence (excitation 480 nm, emission 590 nm) was then measured by Shimadzu RF-5301PC spectrofluorometer (Japan. The amount of paclitaxel incorporated in the cMLV(PTX) and cMLV(Dox+PTX) was determined by C—RP-HPLC chromatography (Beckman Coulter, Brea, Calif.). The cMLV(PTX) and cMLV(Dox+PTX) suspensions were diluted by adding water and acetonitrile to a total volume of 0.5 ml. Extraction of paclitaxel was accomplished by adding 5 ml of tert-butyl methyl ether and vortex-mixing the sample for 1 min. The mixtures were centrifuged and the organic layer was transferred into a glass tube and evaporated to dryness under argon. Buffer A (95% water, 5% acetonitrile) was used to rehydrate the glass tube. To test PTX concentration, 1 ml of the solution was injected into a C18 column, and the paclitaxel was detected at 227 nm (flow rate 1 ml/min). To obtain the release kinetics of Dox and PTX from liposomes, the releasing media was removed from cMLVs incubated in 10% fetal bovine serum (FBS)-containing media at 37° C. and replaced with fresh media daily. The removed media was quantified for Dox fluorescence (by spectrofluorometer) and PTX fluorescence (by HPLC) every day.

In Vitro Drug Loading Efficiency.

Loading efficiency was determined by the ratio of encapsulated drug to total phospholipid mass. Phospholipid phosphate assay was carried out to calculate the phospholipid mass. cMLVs were centrifuged, and 100 μl chloroform was added to the pellets to break down the lipid bilayers. The samples were transferred to glass tubes and evaporated to dryness. After adding 100 μl perchloric acid, the samples were boiled at 190° C. for 25 min. Samples will turn brown then clear as the lipids are digested. Samples were cooled to room temperature and diluted to 1 ml with distilled water. The amount of phospholipid phosphate was determined by the malachite green phosphate detection kit (R&D systems, Minneapolis, Minn.).

In Vitro Cytotoxicity and Data Analysis.

B16-F10 and 4T1 cells were plated at a density of 5×10³ cells per well in 10% fetal bovine serum (FBS)-containing media in 96-well plates and grown for 6 h. The cells were then exposed to a series of concentrations of cMLV (single drug) or cMLV (drug combinations), at different weight ratios of combined drugs, for 48 h. The cell viability was assessed using the Cell Proliferation Kit II (XTT assay) from Roche Applied Science according to the manufacturer's instructions. Cell viability percentage was determined by subtracting absorbance values obtained from media-only wells from drug-treated wells and then normalizing to the control cells without drugs. The fraction of cells affected (fₐ) at each drug concentration was subsequently determined for each well. The data was analyzed by nonlinear regression to get the IC₅₀ value. The Combination Index (CI) values were calculated by the equation: CI=C_{A,X}/IC_{X,A}+C_{B,X}/IC_{X,B}²⁸. Using this analysis method, a CI=0.9−1.1 reflects additive activity, a CI>1.1 indicates antagonism, while a CI<0.9 suggests synergy.

Western Blot Analysis.

Cells were collected 24 h after treatment and lysed in lysis buffer supplemented with protease inhibitors, incubated on ice for 15 min, and then cleared by centrifugation at 10000×g at 4° C. for 10 min. Protein concentration was determined using Micro BCA Protein Assay Kit (Thermo Scientific). Lysates (20 μg) were separated by reducing 12% polyacrylamide gel and then transferred to polyvinylidene difluoride membranes. Immunodetection of ERK was carried out with antibodies specific to rabbit phospho-specific protein p44/42 MAPK (Erk 1/2) and goat anti-rabbit IR Dye®680RD. Immunodetection of β-actin was carried out with antibodies against β-actin and goat anti-mouse IR Dye®800CW. Membranes were developed using Odyssey Infrared Fluorescent Imager (LI-COR BioSciences, Lincoln, Nebr.).

Determination of Doxorubicin and Paclitaxel Levels in Tumor.

BALB/c female mice (6-10 weeks-old) were inoculated subcutaneously with 0.2×10⁶ 4T1 tumor cells. The tumors were allowed to grow for 20 days to a volume of ˜500 mm³ before treatment. On day 20, the mice were injected intravenously through the tail vein with 8.33 mg/kg Dox+1.66 mg/kg PTX, 5 mg/kg Dox+5 mg/kg PTX, 1.66 mg/kg Dox+8.33 mg/kg PTX either in solution or in cMLVs. Three days after injection, tumors were excised and frozen at −20° C. Docetaxel (10 nl, 100 μg/ml) as an internal standard (IS) for paclitaxel, or 10 μl of Daunorubicin (100 μg/ml) as an internal standard for Doxorubicin was added to the weighted tumor tissues. In order to extract paclitaxel and the internal standard (Docetaxel), tumor tissue was homogenized in 1 ml ethyl acetate and then centrifuged at 5000 rpm for 10 min. In order to extract doxorubicin and its internal standard (Daunorubicin), tumor tissue was homogenized in 1 ml methanol and then centrifuged at 5000 rpm for 10 min. Then the organic layer was transferred to a clean glass tube and evaporated to dryness under a stream of argon. Buffer A (95% water, 5% acetonitrile) was used to rehydrate the sample in the glass tube. 1 ml of the solution was injected into C18 column, and the paclitaxel was detected at 227 nm (flow rate 1 ml/min), and doxorubicin was detected at 482 nm (flow rate 1 ml/min). Stock solutions of Dox and PTX (100, 10, and 1 μg/ml) and IS were prepared as calibration samples. Then 500 μl of tumor homogenates were spiked with 500 μl calibration samples with the internal standard at fixed concentration of 1 μg/ml. Calibration curves of doxorubicin and paclitaxel were constructed using the ratio of peak height of doxorubicin or paclitaxel and internal standard by weighted linear regression analysis.

In Vivo Antitumor Activity Study.

BALB/c female mice (6-10 weeks-old) were inoculated subcutaneously with 0.2×10⁶ 4T1 breast tumor cells. The tumors were allowed to grow for 8 days to a volume of ˜50 mm³ before treatment. On day 8, the mice were injected intravenously through the tail vein with 3.33 mg/kg Dox+0.67 mg/kg PTX, 2 mg/kg Dox+2 mg/kg PTX, 0.67 mg/kg Dox+3.33 mg/kg PTX, either in cMLVs or in solution every three days (six mice per group). Tumor growth and body weight were monitored until the end of an experiment. The length and width of the tumor masses were measured with a fine caliper every three days after injection. Tumor volume was expressed as ½×(length×width²). Survival end point was set when the tumor volume reached 1000 mm³. The survival rates are presented as Kaplan-Meier curves. The survival curves of individual groups were compared by a log-rank test.

Immunohistochemistry of Tumors, Cardiac Toxicity and Confocal Imaging.

BALB/c female mice (6-10 weeks-old) were inoculated subcutaneously with 0.2×10⁶ 4T1 tumor cells. The tumors were allowed to grow for 20 days to a volume of ˜500 mm³ before treatment. On day 20, the mice were injected intravenously through tail vein with 8.33 mg/kg Dox+1.66 mg/kg PTX, 5 mg/kg Dox+5 mg/kg PTX, 1.66 mg/kg Dox+8.33 mg/kg PTX in solution or cMLVs. Three days after injection, tumors were excised, fixed, frozen, cryo-sectioned, and mounted onto glass slides. Frozen sections were fixed, and rinsed with cold PBS. After blocking and permealization, the slides were washed by PBS and incubated with TUNEL reaction mixture (Roche, Indianapolis, Ind.) for 1 hr and counterstained with DAPI (Invitrogen, Carlsbad, Calif.). Fluorescence images were acquired by a Yokogawa spinning-disk confocal scanner system (Solamere Technology Group, Salt Lake City, Utah) using a Nikon Eclipse Ti-E microscope. Illumination powers at 405, 491, 561, and 640 nm solid-state laser lines were provided by an AOTF (acousto-optical tunable filter)-controlled laser-merge system with 50 mW for each laser. All images were analyzed using Nikon NIS-Elements software. For quantifying TUNEL positive cells, 4 regions of interest (ROI) were randomly chosen per image at ×2 magnification. Within one region, area of TUNEL-positive nuclei and area of nuclear staining were counted by Nikon NIS-Element software, with data expressed as % total nuclear area stained by TUNEL in the region.

For cardiac toxicity, heart tissues were harvested 3 days after injection, and were fixed in 4% formaldehyde. The tissues were frozen and then cut into sections and mounted onto glass slides. The frozen sections were stained with hematoxylin and eosin. Histopathologic specimens were examined by light microscopy.

Statistics.

The differences between two groups were determined with Student's t test. The differences among three or more groups were determined with a one-way ANOVA.

### Results and Discussions

Characteristics of Combinatorial Drug Delivery Via cMLVs.

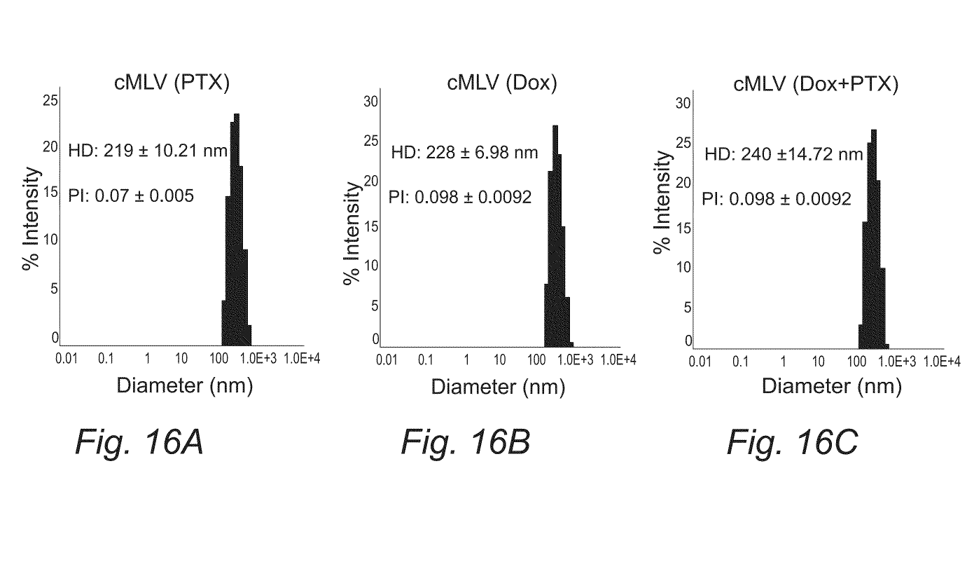

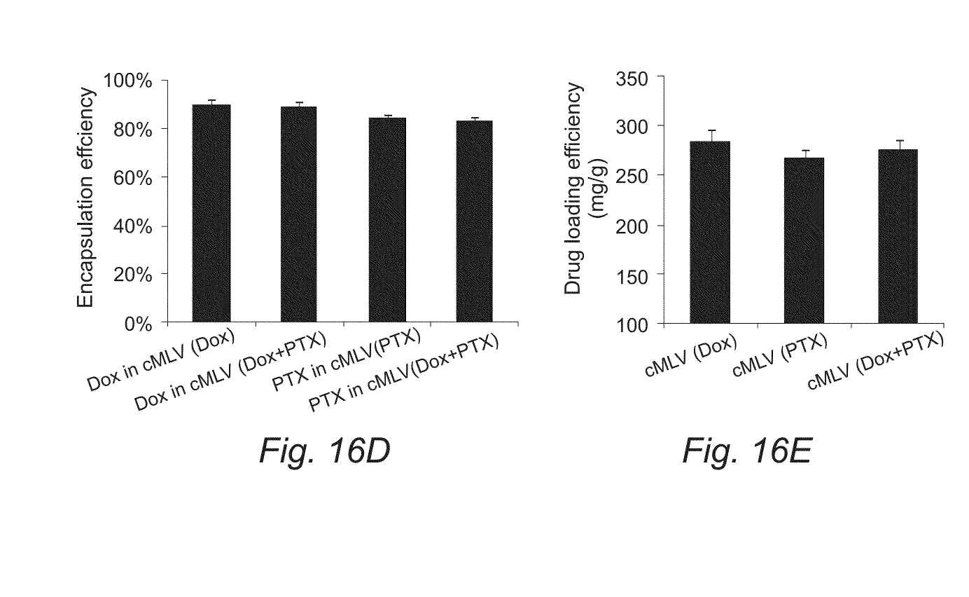

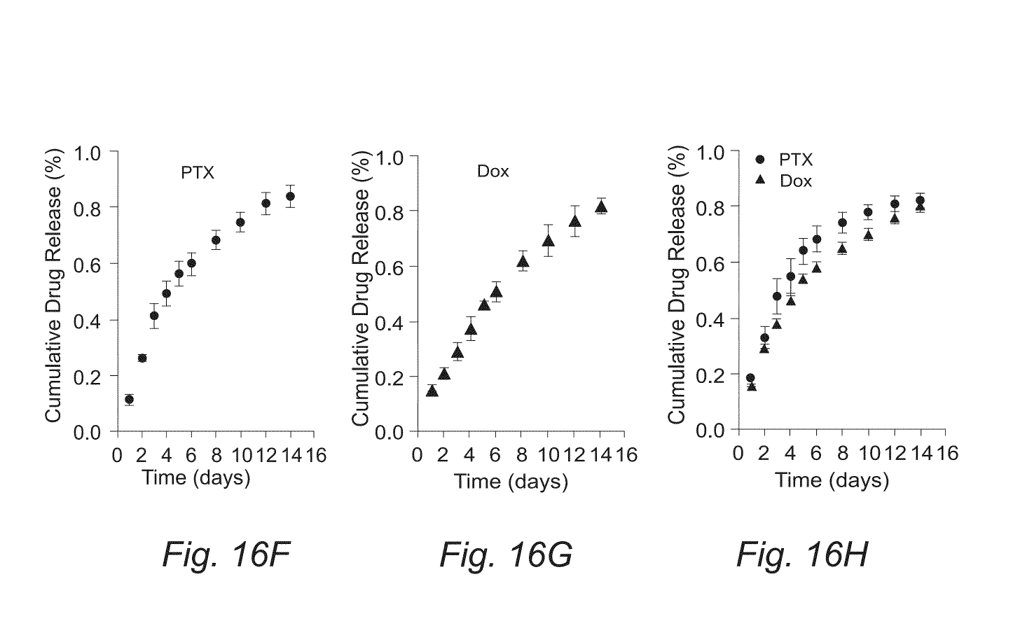

Our strategy of combination drug delivery via crosslinked multilayer liposomal vesicles was to incorporate the hydrophobic drug paclitaxel (PTX) into the lipid membranes and encapsulate the hydrophilic drug doxorubicin (Dox) in the aqueous core of liposomal vesicles, (see FIGS. 1A and 1B). The crosslinked multilamellar liposomal vesicles (cMLVs) were formed by adding MgCl₂ to trigger vesicle fusion, and then stabilized by dithiothreitol (DTT) to form crosslinkers between adjacent liposomal vesicles. The surface of the crosslinked multilayer liposomes was further PEGylated with thiol-terminated PEG, which is known to enhance vesicle stability and elongate the blood circulation half-life. First, we characterized the physical properties of dual drug-loaded cMLVs compared to single drug-loaded cMLVs to determine whether drug combinations could change the physical properties of liposomal formulation. Dynamic light scattering (DLS) measurements showed that the resulting dual drug-loaded cMLVs had a similar average hydrodynamic diameter as single drug-loaded cMLVs (FIG. 16A-C). We found no significant aggregation of particles during the crosslinking process in all three liposomal formulations, as evident by the narrow size distribution and similar polydispersity observed in both dual drug-loaded and single drug-loaded cMLVs. This suggests that the combination of Dox and PTX in a single nanoparticle has a negligible effect on the formation of cMLV particles.

We next determined whether the encapsulation efficiency or loading yield of cMLVs was affected by loading multiple therapeutics. Single drug-loaded and dual drug-loaded cMLVs were dissolved in organic solvents to free all encapsulated drugs (Dox and/or PTX). Dox and PTX concentrations were quantified by spectrofluorometer and/or HPLC, respectively. As shown in FIG. 16D, the drug encapsulation efficiency of Dox and PTX in cMLV(Dox+PTX) was not significantly different from that in either cMLV(Dox) or cMLV(PTX). It was also shown that cMLV(Dox+PTX) had a comparable drug loading yield (˜270 mg drug per g of phospholipids) compared to single drug-loaded cMLVs (FIG. 16E). The drug release profiles of Dox and PTX were also evaluated in dual drug-loaded cMLVs to investigate whether the cMLVs are able to release the individual drugs in a controlled manner. The results of in vitro drug release assay showed that cMLV(Dox+PTX) has slow and linearly sustained release kinetics of both Dox and PTX (up to 2 weeks), similar to that of single drug-loaded cMLVs (FIG. 16F-H). These results confirm that this approach enables the loading of drugs with different hydrophobicity into the same nanoparticles with efficient drug loading yield and sustained drug release profiles.

In Vitro Analysis of Doxorubicin:Paclitaxel for Drug Ratio-Dependent Synergy.

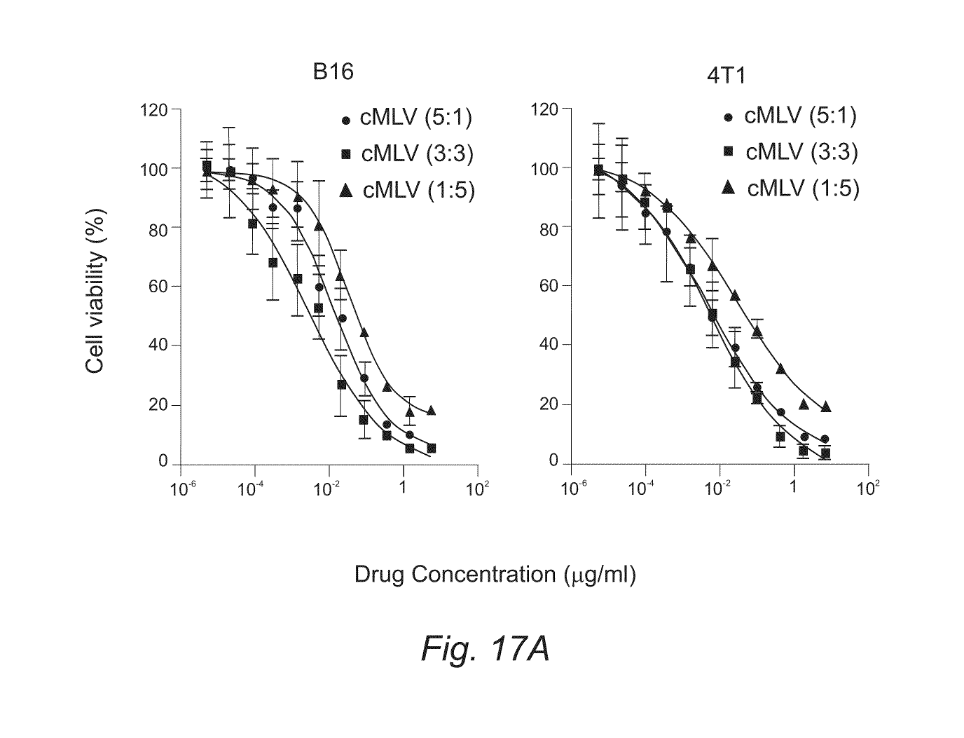

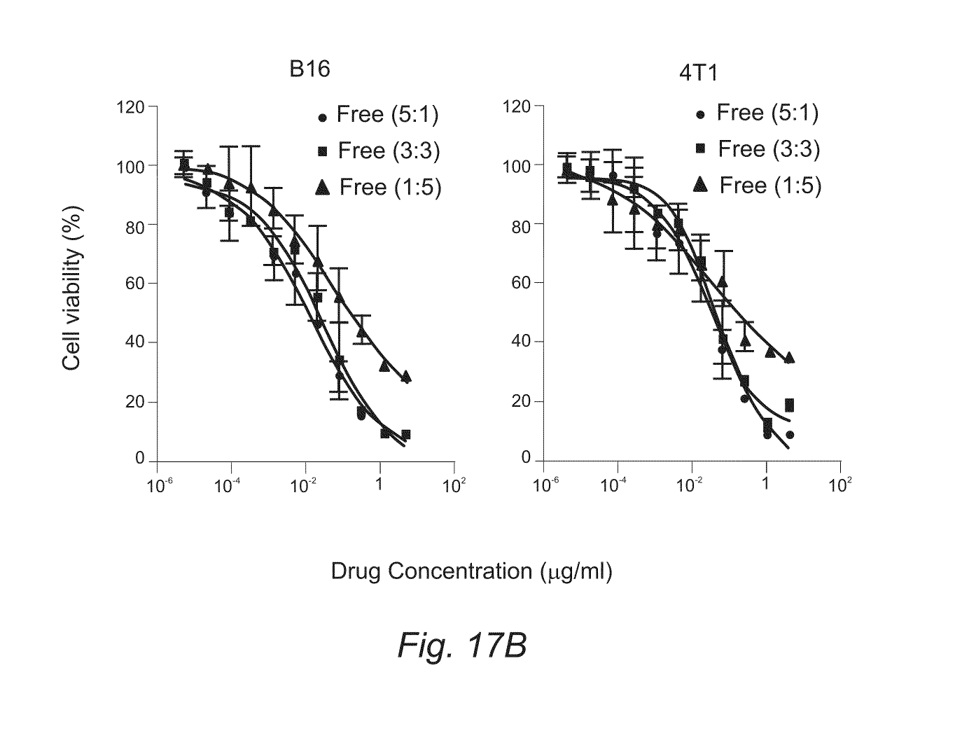

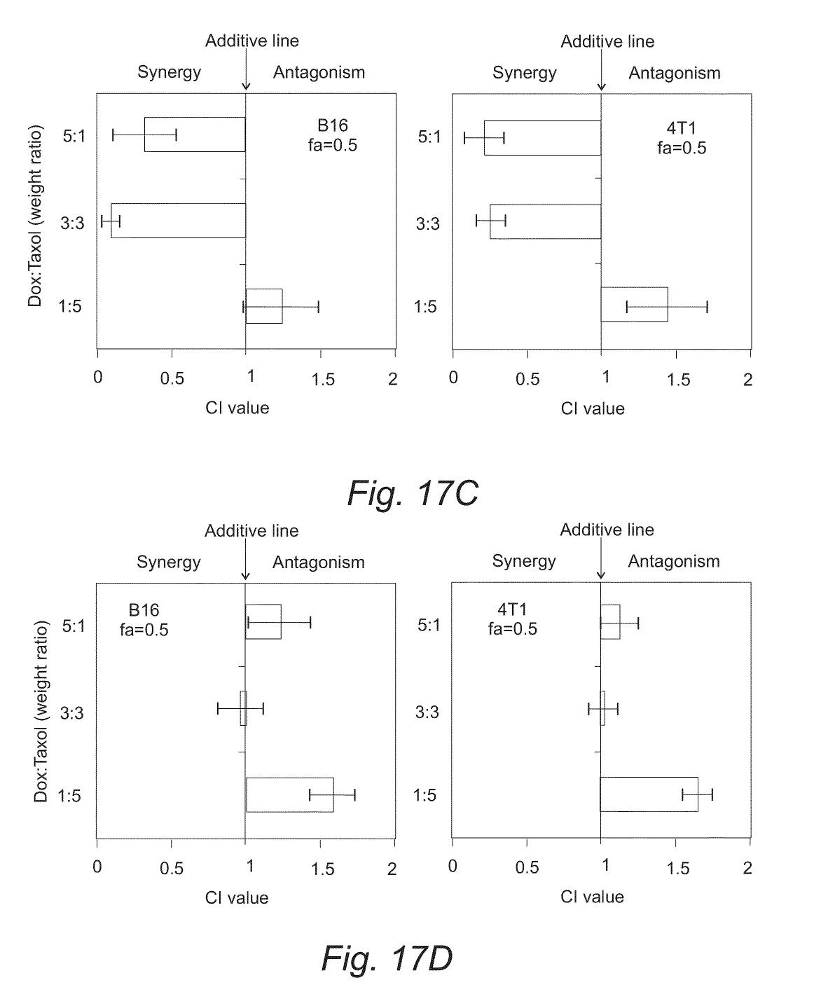

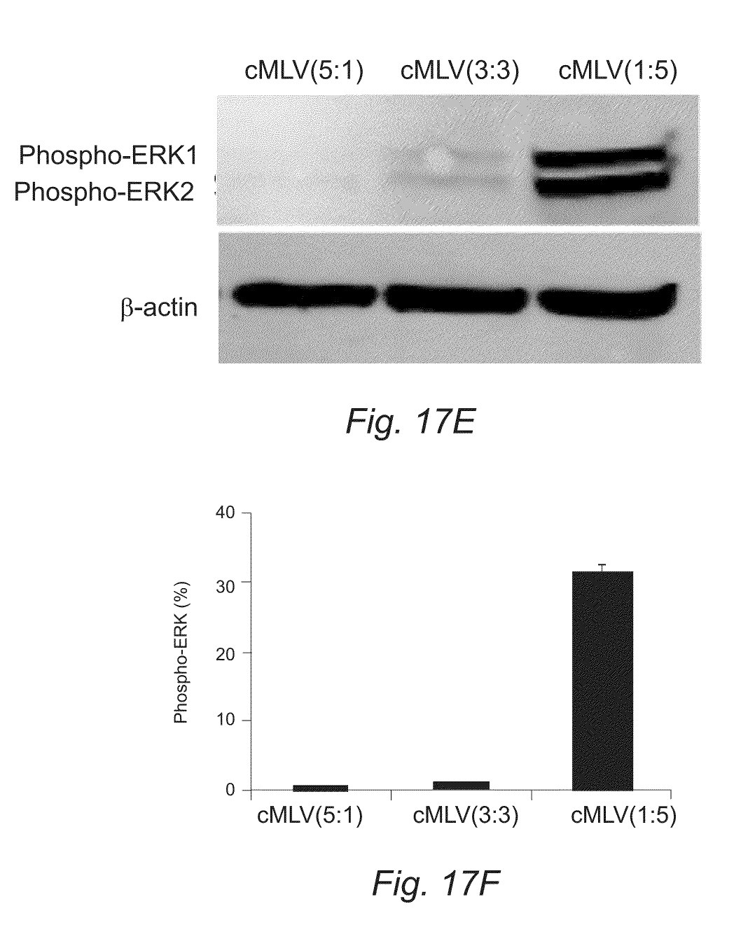

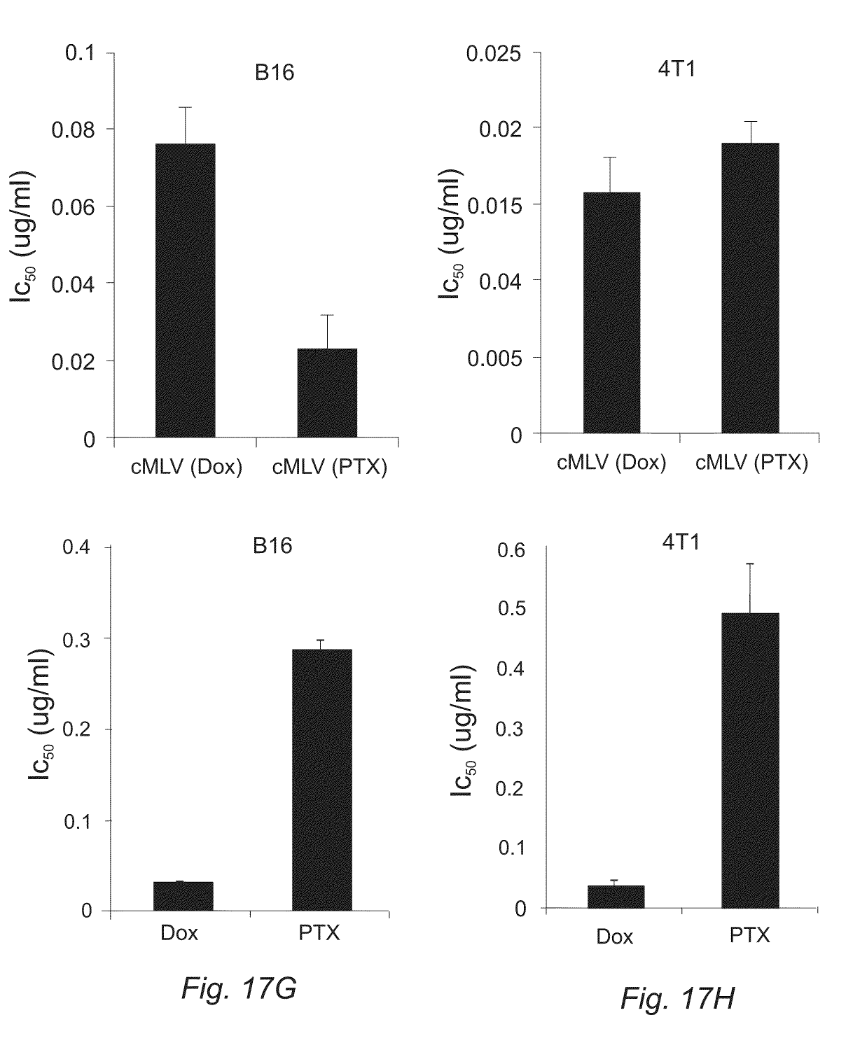

Certain cases of combinatorial drug delivery are able to induce synergistic effects and it has been reported that the combination effect, synergy, additivity, or antagonism, can be affected by the dose ratio. To test this hypothesis, the cytotoxicities of cMLV(Dox+PTX) encapsulating three different drug weight ratios (5:1, 3:3 and 1:5) were examined in B16 and 4T1 cell lines. The cytoxocicities of cMLVs were compared to the cytotoxicities of the same three ratio combinations in cocktail solutions. FIG. 17A summarizes the results of IC₅₀ measurements of the dual drug-loaded cMLVs with the three different dose ratios after 48 h of incubation with B16 and 4T1 cells. The IC₅₀ values of cMLV (Dox+PTX) at Dox:PTX ratios of 3:3 and 5:1 were significantly smaller than that of the 1:5 ratio in the cell lines studied. A similar trend of IC₅₀ values at the different dose ratios was observed for free Dox and PTX combinations (FIG. 17B).

Moreover, combination index (CI) values were analyzed from in vitro cytotoxicity curves for Dox and PTX combinations either in cMLVs or cocktail solutions to assess the effects of combination. The IC₅₀ values of individual drugs either in cMLVs or in solution are shown in FIGS. 17G-H. A CI of less than, equal to, and greater than 1 is known to indicate synergy, additivity, and antagonism, respectively. Although combination indexes are only shown for a 0.5 fraction of affected cells (fa) (50% cell growth inhibition relative to control cells) in FIG. 17, the profile of synergy/antagonism was similar for other fa values. As shown in FIG. 17C, at fa=0.5, synergistic effects were observed in both B16 and 4T1 tumor cells for co-loaded cMLVs at Dox:PTX ratios of 5:1 and 3:3 (Dox:PTX), while the combination at a 1:5 ratio was additive or antagonistic in B16 and 4T1 cells. In contrast, no synergistic effect was observed in B16 or 4T1 cells treated with three ratios of Dox and PTX in cocktail, as shown in FIG. 17D, further confirming the potential of cMLVs to induce synergy by controlling dose ratios.

Our data indicated that combinatorial delivery via cMLVs with high ratio of PTX induced additivity or antagonism. In fact, some studies have shown that low concentrations of PTX can induce cell apoptosis more effectively than high concentrations, but the mechanism remains elusive. Further studies suggested that PTX could activate the extracellular signal regulated kinase (ERK), leading to cell proliferation and building drug resistance. It was also shown that inhibiting the ERK pathway dramatically enhanced cell apoptosis induced by PTX. These studies indicate that the high PTX concentration could be responsible for the antagonism seen between Dox and PTX at a 1:5 dose ratio. To investigate whether there is a difference in activation of ERK in melanoma cells treated by cMLV(Dox+PTX) at the three different dose ratios, phosphorylated ERK expression was detected by western blot. As shown in FIG. 17E, the combination of Dox and PTX at a 1:5 ratio showed significantly increased expression of phosphorylated ERK compared to the 3:3 and 5:1 ratios. Quantification of ERK phosphorylation (FIG. 17F) showed a 30-fold enhancement in phosphorylated ERK in cells treated by cMLV(Dox+PTX) 1:5 ratio. This data suggests that ratio-dependent combination effects are likely linked to the ERK activation caused by high concentrations of PTX.

The IC₅₀ values of individual drugs either in cMLVs or in solution for B16 melanoma cells and 4T1 breast cancer cells are shown in FIGS. 17 G and H, respectively.

Drug Ratio-Dependent Efficacy of cMLV(Dox+PTX) in Tumor Treatment.

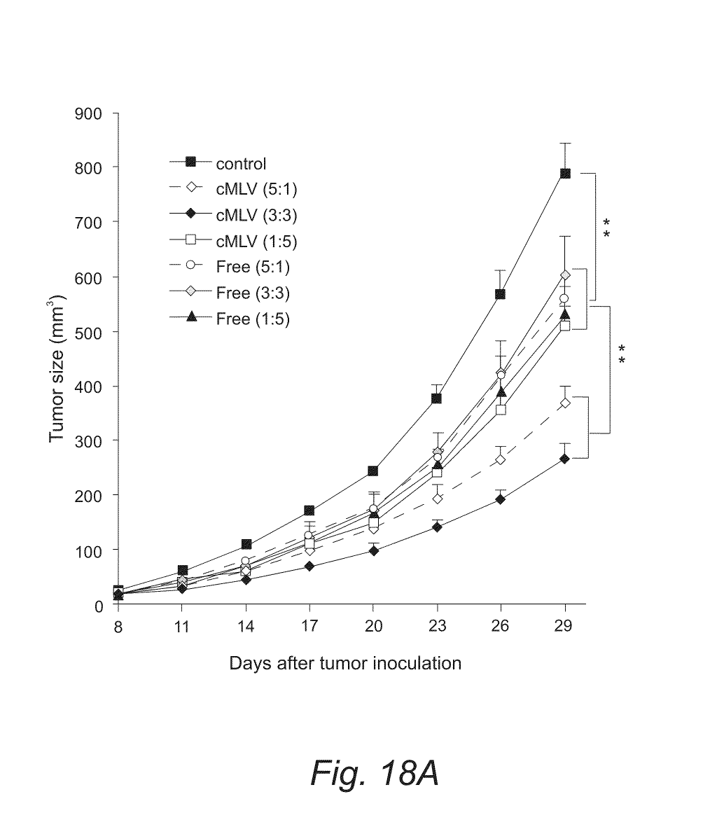

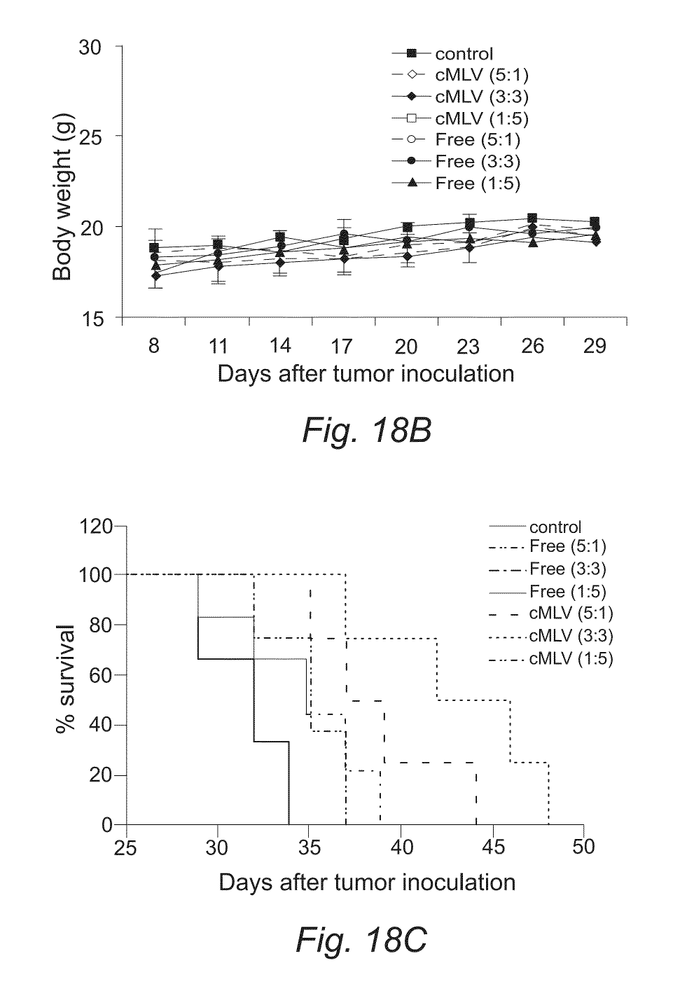

In order to assess whether the drug ratio-dependent in vitro cytotoxicity was also manifested in vivo, doxorubicin and paclitaxel were co-encapsulated in cMLV particles at a weight ratios ranging from 5:1 to 1:5, while keeping the total drug mass encapsulated in cMLVs constant. This panel of fixed ratio cMLV formulations and the same fixed ratio combination in cocktail solutions were evaluated for their antitumor efficacy in an in vivo 4T1 breast tumor model. As shown in FIG. 18A, tumor volume in the groups treated with drug combinations in solution decreased significantly compared to that in the control group (p<0.01). Tumor volume between the groups treated with different ratios of drug combinations in solution did not show a significant difference (p>0.05), consistent with the in vitro finding that free drug combinations did not show a synergistic effect. In comparison, administration of the 5:1 and 3:3 weight ratio of Dox to PTX in cMLV resulted in significantly enhanced antitumor activity compared to the 1:5 ratio, indicating the ability of cMLVs to induce a ratio-dependent synergistic effect in vivo. Moreover, no weight loss was observed for all treated groups during the experiment (FIG. 18B), indicating that there was no significant toxicity from these dose combinations.

The dose-dependent antitumor activity was further confirmed by survival test as shown in FIG. 18C. Treatment with three ratios of drug combinations in cocktail solutions resulted in an increased survival time (35 days) compared to PBS treatment (28 days, p<0.05). Administration of the 5:1 and 3:3 weight ratios in cMLV formulations resulted in a significant increased life span compared to 1:5 ratio in cMLVs (p<0.05). These results confirmed a dose-dependent synergy of drug combinations in cMLV formulations and provide a positive correlation linking the combination effects in vitro to the degree of antitumor efficacy in vivo.

Drug Ratio-Dependent Efficacy of Co-Encapsulated Dox:PTX on Tumor Apoptosis.

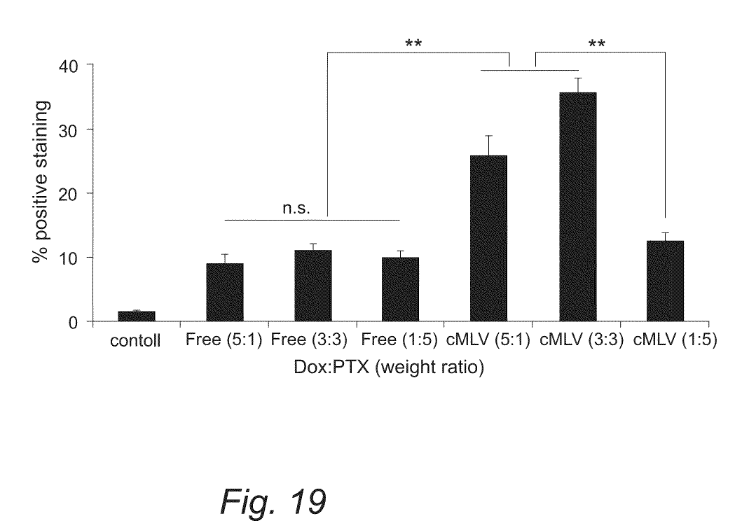

To investigate the ratio-dependent antitumor mechanism in vivo, TUNEL assay was performed to detect apoptotic cells in 4T1 tumors treated with different ratios of Dox and PTX in cocktail and in cMLV formulations for 3 days. 4T1 tumors treated with three different ratios (5:1, 3:3 and 1:5) of Dox and PTX in solution induced cell apoptosis by a significant amount compared to controls. The apoptosis index was not remarkably different among different ratios of drug combination cocktails (p>0.05), consistent with the similar effect on tumor growth between the cocktail treatments. Moreover, the 5:1 and 3:3 ratio of Dox and PTX in cMLVs promoted tumor cell apoptosis compared to the antagonistic ratio (1:5). The quantified data (FIG. 19A) further confirms that drug ratio-dependent antitumor efficacy via cMLVs can contribute to different levels of tumor apoptosis.

In Vivo Cardiac Toxicity Evaluation of Drug Combinations in cMLV Formulations.

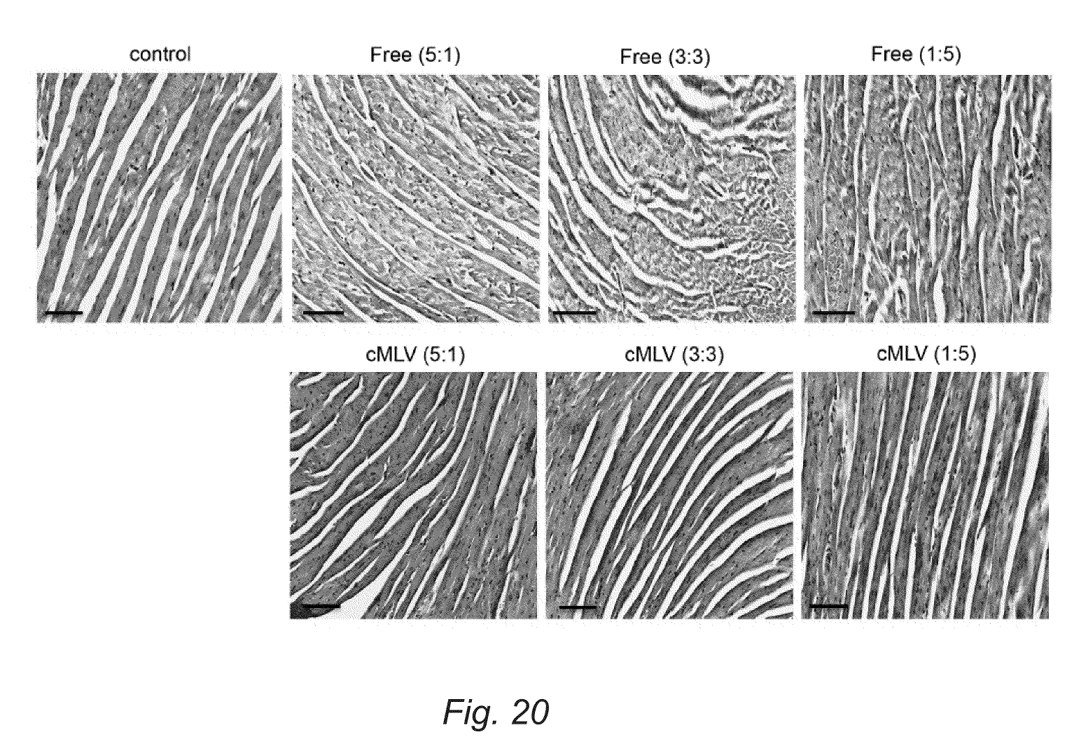

An unexpected clinical outcome of increased cardiotoxicity after combined treatments of Dox and PTX has been reported, thus limiting their clinical applications. To investigate whether the synergistic therapies could induce synergistic cardiac toxicity, three weight ratios of Dox and PTX in both cMLV formulations and cocktail solutions were evaluated for cardiac effects. Mice bearing 4T1 tumors were injected intravenously through tail vein with 8.33 mg/kg Dox+1.66 mg/kg PTX, 5 mg/kg Dox+5 mg/kg PTX, 1.66 mg/kg Dox+8.33 mg/kg PTX in solution or in cMLVs. Hematoxylin and eosin staining of cardiac tissue sections from each treatment group were examined. As shown in FIG. 20, all three dose ratios of Dox and PTX in cocktail solutions caused damage to cardiac tissue indicated by myofibrillary loss, disarray, and cytoplasmic vacuolization. No significant histopathologic changes in cardiac tissue were observed in three dose ratios of Dox and PTX in cMLV formulations compared to the control group, indicating that a reduction in systemic toxicity can be achieved when drugs are co-encapsulated in cMLVs. Moreover, no synergistic toxicity was observed in the synergistic ratios (5:1 and 3:3) of Dox and PTX in cMLVs.

In Vivo Maintenance of Drug Ratios in cMLV Formulations.

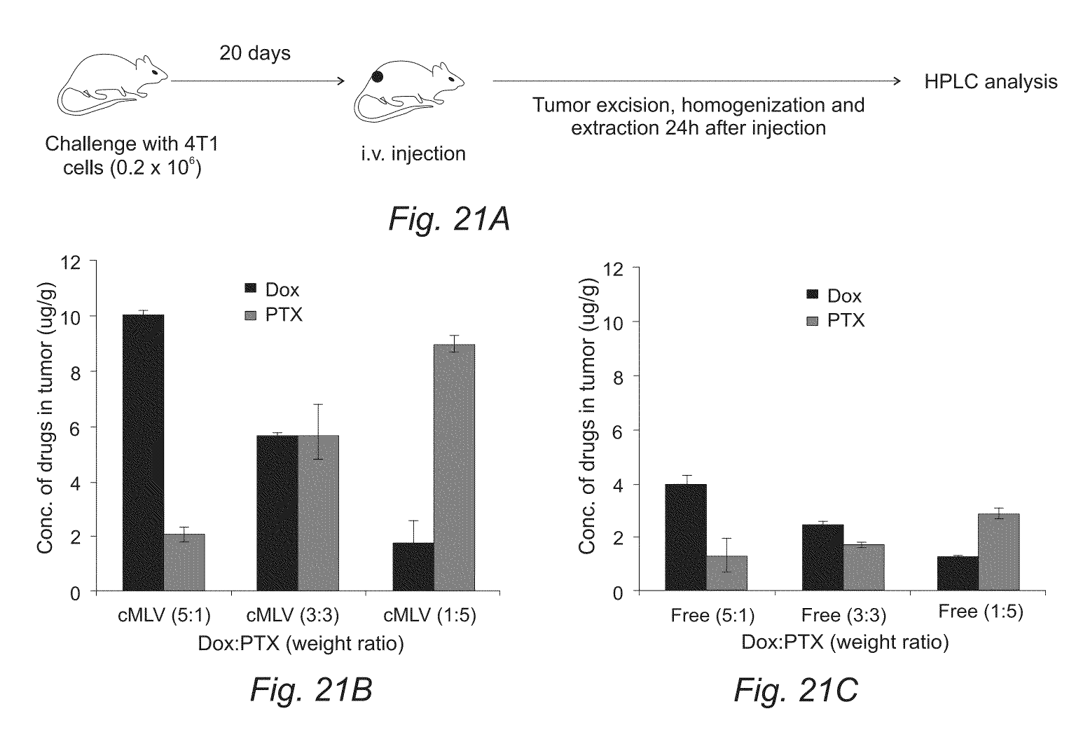

In order to determine if dose ratios of drugs delivered via cMLVs were well maintained in vivo, and to correlate the in vivo effects to the in vitro combination effect, the drug concentrations in tumor tissues were measured. Dox and PTX were co-encapsulated at the 5:1, 3:3, and 1:5 weight ratios inside cMLVs, and administered i.v. to mice, while the same ratios of drug combinations in cocktail solutions were administrated as controls. Twenty-four hours after injection, tumors were excised and homogenized, and Dox and PTX were extracted and detected by HPLC analysis, as illustrated in FIG. 21A. The HPLC results show that cMLVs maintain the Dox:PTX weight ratio at 5:1, 3:3 and 1:5, respectively, in tumors for over 24 h (FIG. 21B). In comparison, the free-drug cocktail Dox:PTX weight ratio changed dramatically after administration, shown in FIG. 21C. In addition, remarkably more Dox and PTX accumulated in tumors when administered via cMLV formulations compared to free-drug cocktails with equivalent amounts of Dox and PTX, thus maximizing their combinatorial effect. These results indicate that cMLVs can efficiently maintain dose ratio in vivo, thus translating the combination effects (synergy, additivity and antagonism) from in vitro to in vivo.

To summarize, a robust approach for combinatorial chemotherapy was presented by encapsulating two different types of antitumor therapeutics, with ratiometric control over drug loading, into a crosslinked multilamellar liposomal formulation. Previously, we have demonstrated the superior ability of cMLVs as drug carriers to offer controllable and sustainable drug release profiles of Dox with increased vesicle stability, enabling improved antitumor activity. In the present study, we explore the potential of cMLVs in combinatorial delivery of Dox and PTX, which have been widely used as a combined anthracycline-taxane regimen in metastatic breast cancer, to achieve synergistic antitumor activity. A number of studies suggest the non-coordinated biodistribution profiles of this combination when administered in cocktail solutions limit the efficacy of the combination. However, the versatile crosslinked multilamellar liposomes enabled codelivery of Dox and PTX via a single vesicle to the cancer site, thus coordinating the plasma elimination and tissue distribution of the combined drugs.

Recent studies revealed that the activity of antitumor drug combinations is determined by the ratio of the combined drugs exposed to cells. Therefore, it is highly desirable to maintain a synergistic ratio of combined drugs in vivo. Here, we demonstrate that the stability of cMLVs enables us to co-load Dox and PTX with predefined ratios and induce a ratio-dependent synergy in tumor cells. It was previously reported by a number of studies that PTX-containing liposomes could not maintain stability over a drug-to-lipid molar ratio of 3%-4%. For example, one study showed that more than 8% PTX-to-lipid formulations (PG:PC 3:7 molar ratio) were not stable for one day²⁴. cMLVs can maintain high stability up to 30% PTX-to-lipid molar ratio. This is most likely due to the crosslinked multilamellar structure of cMLVs, which allows co-delivery of Dox and PTX with high loading efficiency. In addition, enhanced vesicle stability of cMLVs enables these nanoparticles to maintain the dose ratios of Dox and PTX at tumor sites, translating the ratio-dependent synergy from in vitro to in vivo. This would be beneficial for predicting the efficacy of treatment in clinical trials and the optimal design of combination therapy based on in vitro cellular experiments. Our in vivo results also reveal that the enhanced combinatorial efficacy of cMLVs compared to cocktail combination is due to the augmented accumulation of drugs at tumor sites.

In clinical studies, Dox and PTX exhibit an increased cardiac toxicity when combined in cocktail^{40, 41}, raising the concern that a significant side effects could be associated with the synergistic therapeutic efficacy. However, we previously demonstrated that the robust cMLV formulation greatly reduced systemic toxicity of Dox, most likely due to the sustained drug release profile of Dox. Here, we show that cMLVs can induce synergistic effects on tumor growth without causing cardiac toxicity, further demonstrating their potential in combinatorial drug delivery. These results, taken together, indicated that the superior ability of cMLVs in combination therapy is not only attributed to the prolonged exposure of drugs to tumor cells, but also to the maintenance of synergistic dose ratios at the site of action with no significant systemic toxicity.

### 4. Codelivery of Doxorubicin and Paclitaxel Via Crosslinked Liposomal Formulations to Overcome Multidrug Resistance in Tumor

### Materials and Methods

Mice.

Female BALB/c mice (6-10 weeks old) were purchased from Charles River Breeding Laboratories (Wilmington, Mass.). All mice were held under specific pathogen-reduced conditions in the Animal Facility of the University of Southern California (USA). All experiments were performed in accordance with the guidelines set by the National Institutes of Health and the University of Southern California on the Care and Use of Animals.

Cell Culture.

B16 tumor cells (B16-F10, ATCC number: CRL-6475) and 4T1 tumor cells (ATCC number: CRL-2539) were maintained in a 5% CO₂ environment with Dulbecco's modified Eagle's medium (Mediatech, Inc., Manassas, Va.) supplemented with 10% FBS (Sigma-Aldrich, St. Louis, Mo.) and 2 mM of L-glutamine (Hyclone Laboratories, Inc., Omaha, Nebr.). B16-R and 4T1-R cells were produced by continuously treating B16 and 4T1 cells with 5 μg/ml PTX for 4 days. The cells were then recovered by replacing medium with fresh medium without drugs for 7 days. The remaining cells formed drug resistance for PTX. JC cells (ATCC number: CRL-2116) were used as a model drug-resistant tumor cell line because it has been shown that JC cells overexpress P-gp and exhibit a drug-resistant phenotype, both in vitro and in vivo.

Synthesis of cMLVs.

Liposomes were prepared based on the conventional dehydration-rehydration method. All lipids were obtained from the NOF Corporation (Japan). 1.5 μmol of lipids 1,2-dioleoyl-sn-glycero-3-phosphocholine (DOPC), 1,2-dioleoyl-sn-glycero-3-phospho-(1′-rac-glycerol) (DOPG), and maleimide-headgrouplipid-1,2-dioleoyl-sn-glycero-3-phosphoeth-anolamine-N-[4-(p-maleimidophenyl) butyramide (MPB-PE) were combined in chloroform at a molar lipid ratio of DOPC:DOPG:MPB=4:1:5, and the organic solvent in the lipid mixture was evaporated under argon gas. The lipid mixture was further dried under vacuum overnight to form dried thin lipid films. To prepare cMLV(PTX) and cMLV(Dox+PTX) at a molar ratio of 0.2:1 (drugs:lipids), paclitaxel in organic solvent was mixed with the lipid mixture to form dried thin lipid films. The resultant dried film was hydrated in 10 mM Bis-Tris propane at pH 7.0 with (cMLV(Dox) or cMLV(Dox+PTX)) or without doxorubicin (cMLV(PTX)) at a molar ratio of 0.2:1 (drugs:lipids) with vigorous vortexing every 10 min for 1 h, followed by applying 4 cycles of 15-s sonication (Misonix Microson XL2000, Farmingdale, N.Y.) on ice in 1-min intervals of each cycle. To induce divalent-triggered vesicle fusion, MgCl₂ was added at a final concentration of 10 mM. The resulting multilamellar vesicles were further crosslinked by addition of Dithiothreitol (DTT, Sigma-Aldrich) at a final concentration of 1.5 mM for 1 h at 37° C. The resulting vesicles were collected by centrifugation at 14,000 g for 4 min and then washed twice with PBS. For pegylation of cMLVs, the particles were incubated with 1 μmol of 2 kDa PEG-SH (Laysan Bio Inc., Arab, Ala.) for 1 h at 37° C. The particles were then centrifuged and washed twice with PBS. The final products were stored in PBS at 4° C.

In Vitro Cytotoxicity and Data Analysis.

B16-F10, 4T1, B16-R, 4T1-R, and JC cells were plated at a density of 5×10³ cells per well in D10 media in 96-well plates and grown for 6 h. The cells were then exposed to a series of concentrations of cMLV (single drug) or cMLV (drug combinations) for 48 h. The cell viability was assessed using the Cell Proliferation Kit II (XTT assay) from Roche Applied Science according to the manufacturer's instructions. Slope m and IC₅₀ were obtained from median effect model, and IIP_{Cmax} was calculated via the following equation: IIP_{Cmax}=log(1+(Cmax/IC₅₀)^{m}). Cmax is the maximum plasma drug concentrations for the commonly recommended dose for each drug.

Cellular Uptake of Doxorubicin and Paclitaxel in Cells.

4T1 cells were seeded in 24-well plates at a density of 2×10⁵ cells per well and grown overnight. The cells were then exposed to cMLV(Dox), cMLV(PTX), cMLV(Dox+PTX), and Dox+PTX. The final concentrations of Dox and PTX were 1 μg/ml for each group. JC cells were seeded at a density of 10⁵ cells per well in D10 media in 96-well plates. The cells were exposed to cMLV(Dox), cMLV(PTX), cMLV(Dox+PTX), and Dox+PTX. The final concentrations of Dox and PTX were 5 μg/ml for each group. At 48 h after treatment, the cells were washed twice with PBS and lysed with PBS containing 1% Triton X-100. Doxorubicin and paclitaxel in cell lysates were extracted by 1:1 (v/v) Chloroform/isopropyl alcohol or ethyl acetate, respectively. Paclitaxel concentrations in cell lysates were measured by HPLC C18 column and detected at 227 nm (flow rate 1 ml/min), and doxorubicin was detected by fluorescence with 480/550 nm excitation/emission. The concentrations of Dox and PTX were normalized for protein content as measured with BCA assay (Pierce).

In Vivo Antitumor Activity Study.

BALB/c female mice (6-10 weeks old) were inoculated subcutaneously with 0.2×10⁶ 4T1 breast tumor cells. The tumors were allowed to grow for 8 days to a volume of ˜50 mm³ before treatment. After 8 days, the mice were injected intravenously through the tail vein with cMLV(2 mg/kg Dox), cMLV(2 mg/kg PTX), and cMLV(2 mg/kg Dox+2 mg/kg PTX) every three days (six mice per group). Tumor growth and body weight were monitored for 40 days or to the end of the experiment. The length and width of the tumor masses were measured with a fine caliper every three days after injection. Tumor volume was expressed as ½×(length×width²). Survival end point was set when the tumor volume reached 1000 mm³. The survival rates are presented as Kaplan-Meier curves. The survival curves of individual groups were compared by a log-rank test.

Immunohistochemistry of Tumors and Confocal Imaging.

BALB/c female mice (6-10 weeks old) were inoculated subcutaneously with 0.2×10⁶ 4T1 or JC tumor cells. The tumors were allowed to grow for 20 days to a volume of ˜500 mm³ before treatment. On day 20, the mice were injected intravenously through the tail vein with cMLV (5 mg/kg Dox), cMLV(5 mg/kg PTX), 5 mg/kg Dox+5 mg/kg PTX, or cMLV(5 mg/kg Dox+5 mg/kg PTX). Three days after injection, tumors were excised, fixed, frozen, cryo-sectioned, and mounted onto glass slides. Frozen sections were fixed and rinsed with cold PBS. After blocking and permeabilization, the slides were washed by PBS and then incubated with TUNEL reaction mixture (Roche, Indianapolis, Ind.) for 1 h. For P-gp expression, the slides were stained after permeabilization with mouse monoclonal anti-P-gp antibody (Abcam, Cambridge, Mass.) for 1 h, followed by staining with Alexa488-conjugated goat anti-mouse immunoglobulin G (IgG) antibody (Invitrogen, Carlsbad, Calif.) and counterstaining with DAPI (Invitrogen, Carlsbad, Calif.). Fluorescence images were acquired by a Yokogawa spinning-disk confocal scanner system (Solamere Technology Group, Salt Lake City, Utah), using a Nikon Eclipse Ti-E microscope. Illumination powers at 405, 491, 561, and 640 nm solid-state laser lines were provided by an AOTF (acousto-optical tunable filter)-controlled laser-merge system with 50 mW for each laser. All images were analyzed using Nikon NIS-Elements software. To quantify TUNEL and P-gp-positive cells, 4 regions of interest (ROI) were randomly chosen per image at ×2 magnification. Within one region, area of TUNEL, or P-gp-positive nuclei, and area of nuclear staining were counted by Nikon NIS-Element software. The data are expressed as % total nuclear area stained by TUNEL or P-gp in the region.

Hematoxylin and Eosin Staining of Heart Sections.

Mice bearing 4T1 tumors were i.v. injected with 5 mg/kg Dox+5 mg/kg PTX or cMLV(5 mg/kg Dox+5 mg/kg PTX). Three days after injection, heart tissues were harvested and fixed in 4% formaldehyde. The tissues were frozen, cut into sections, and mounted onto glass slides. The frozen sections were stained with hematoxylin and eosin. Histopathologic specimens were examined by light microscopy.

Statistics.

Differences between two groups were determined with Student's t test. The differences among three or more groups were determined with a one-way ANOVA.

### Results

In Vitro Efficacy Study by XTT Assay.

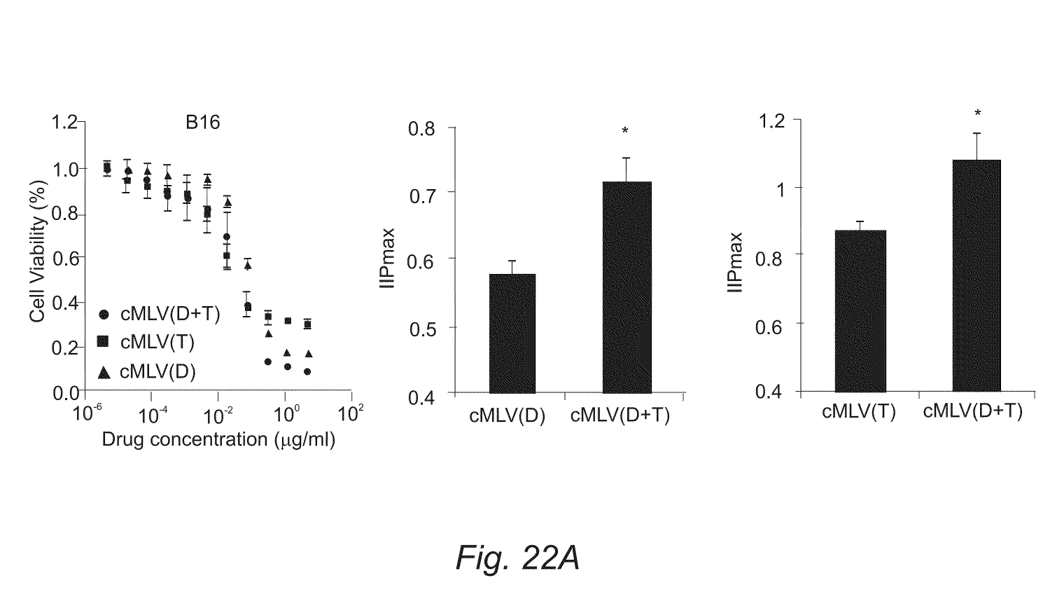

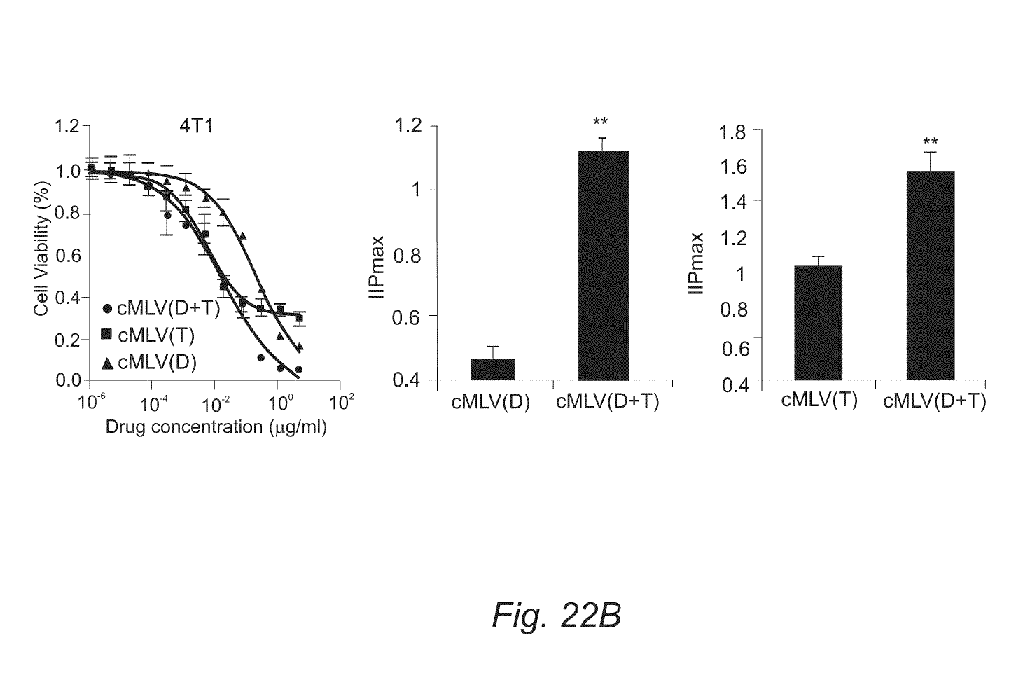

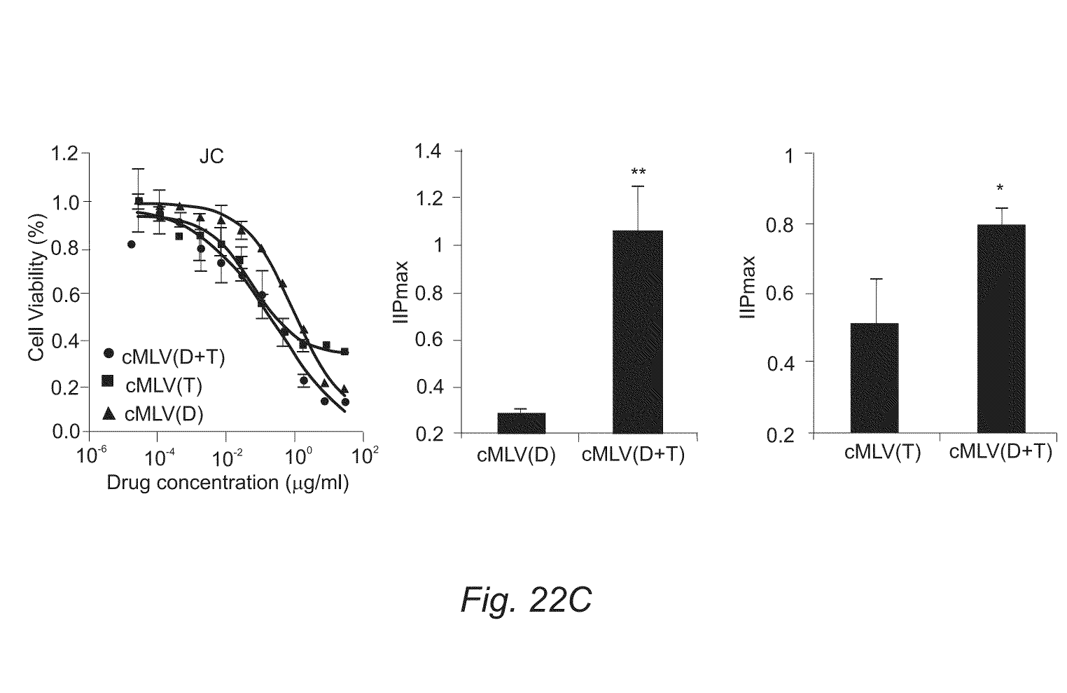

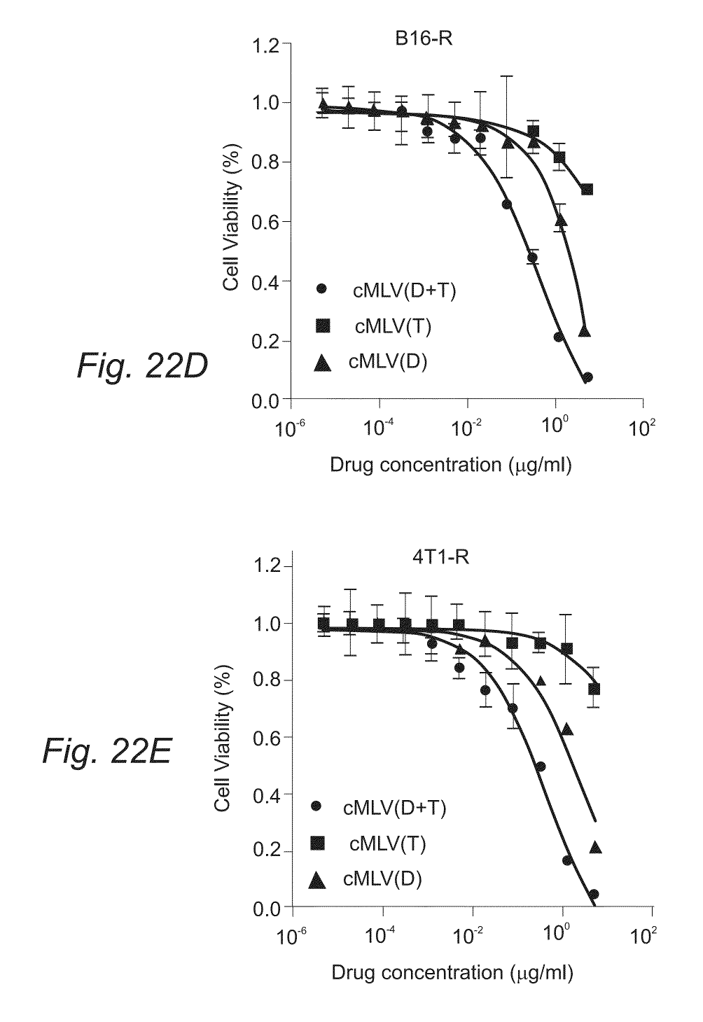

To achieve combination delivery of doxorubicin (Dox) and paclitaxel (PTX), a previously developed crosslinked multilamellar liposomal vesicle (cMLV) was used to incorporate PTX in the lipid membrane and encapsulate Dox in the aqueous core at a 1:1 ratio to form cMLV(Dox+PTX). It has been reported that drug combinations can overcome drug resistance that would otherwise limit the potential application of various monotherapeutics. To determine whether codelivery of Dox and PTX could overcome drug resistance, an in vitro cytotoxicity assay was performed at a wide range of concentrations of single drug-loaded or dual drug-loaded cMLVs. As shown in FIGS. 22A and 22B (left panel), both B16 cells and 4T1 cells developed drug resistance to single drug-loaded cMLVs, but this resistance was inhibited by applying the combined formulation, cMLV(Dox+PTX). The maximal cytotoxicity of single drug-loaded cMLV observed in these two tumor cells was between 60%-80%, while cells treated with dual drug-loaded cMLV(Dox+PTX) showed significantly more growth inhibition.

To further confirm the efficiency of dual drug-loaded cMLVs in overcoming drug resistance, drug-resistant cell lines B16-R and 4T1-R were generated by continuously treating parental B16 or 4T1 with a high concentration of paclitaxel (5 μg/ml). Various concentrations of single drug-loaded cMLV and dual drug-loaded cMLV(Dox+PTX) were incubated with these two drug-resistant cell lines for 48 h, and the cytotoxicity was measured by a standard XTT assay. As shown in FIGS. 22D and 22E, both B16-R and 4T1-R cells showed a high tolerance when treated with cMLV(PTX) or cMLV(Dox), indicating that multidrug resistance had been developed in these cells. In contrast, cMLV(Dox+PTX) triggered significantly more cell death (90-100%) compared to that of single drug-loaded cMLVs, confirming that a codelivery system could overcome drug resistance induced by a high concentration of single drug. Furthermore, in vitro cytotoxicity studies demonstrated therapeutic efficacy of cMLV(Dox+PTX) in JC cells, a model drug-resistant tumor cell line, corroborating the weaker potency of single drug-loaded cMLVs compared to the dual drug-loaded cMLVs. As shown in FIG. 22C (left panel), the maximal cytotoxicity of cMLV(Dox) and cMLV(PTX) was in the range of 60-70%, while peak cMLV(Dox+PTX) cytotoxicity was about 90% in JC cells.

IC₅₀, which indicates drug concentration that causes 50% inhibitory effect on cell proliferation, can provide information on the efficacy of drugs. However, it has also been reported that slope m, a parameter mathematically analogous to the Hill coefficient, may also have a significant effect on cytotoxicity. Therefore, a new model has been developed to evaluate drug activity by incorporating three parameters (IC₅₀, drug concentration, and m) from the median effect model into a single-value IIP (potential inhibition) with an intuitive meaning, i.e., the log reduction in inhibitory effect. Accordingly, to increase the trustworthiness of our experiment, IIP was used to evaluate the efficiency of dual drug-loaded cMLVs on cell viability. As shown in FIG. 22A to 22C (middle and right panels), Dox and PTX in the dual drug-loaded cMLVs displayed a significantly larger IIP_{Cmax} value in the cell lines studied compared to that of the single drug-loaded cMLVs, indicating that combinatorial cMLVs were more potent in cancer treatment than single drug-loaded cMLVs.

Cellular Uptake Study of Doxorubicin and Paclitaxel.

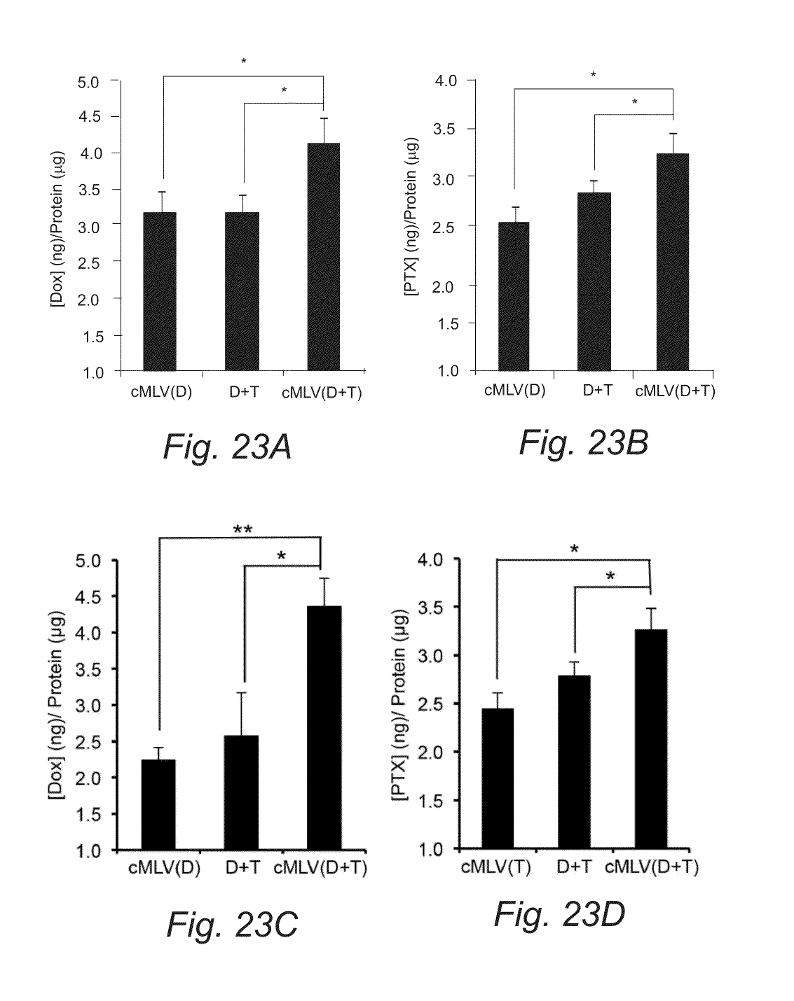

To investigate the mechanism of enhanced cytotoxicity observed with cMLV combination therapy, we evaluated the effect of dual drug-loaded cMLVs on rates of drug influx/efflux in cells. The intracellular accumulation of Dox and PTX was examined by HPLC in 4T1 cells following exposure to Dox (1 μg/ml) and PTX (1 μg/ml) in cMLVs, both individually and in combination, and in JC cells with higher dose of Dox and PTX (5 μg/ml). After 3 h incubation, the extracellular medium was discarded, and intracellular drug (Dox or PTX) accumulation was quantitatively determined by drug concentration in the cell lysates, normalized by total cellular protein content of the cells. As seen in FIGS. 23A and 23B, cMLV(Dox+PTX) significantly increased both Dox and PTX accumulation in 4T1 cells compared to that of single drug-loaded cMLVs (p<0.05), suggesting that combination treatments may overcome drug resistance. In addition, compared to the administration of drug in solution, cMLV combination treatment resulted in higher cellular accumulation of Dox and PTX, an outcome most likely resulting from the internalization of cMLVs by cells through endocytosis (Joo et al., 2013) and, consequently, effectively bypassing the P-gp efflux pumps. The enhanced cellular accumulation of drugs in dual drug-loaded cMLVs was also observed in drug-resistant JC cells (FIGS. 23C and 23D) compared to single drug-loaded cMLVs and drug combination in solution. These data suggest that cMLV(Dox+PTX) significantly enhanced the intracellular accumulation of anticancer drugs through mechanisms involving both combination treatment and nanoparticle delivery.

Effect of Codelivered Nanoparticles on P-Gp Expression.

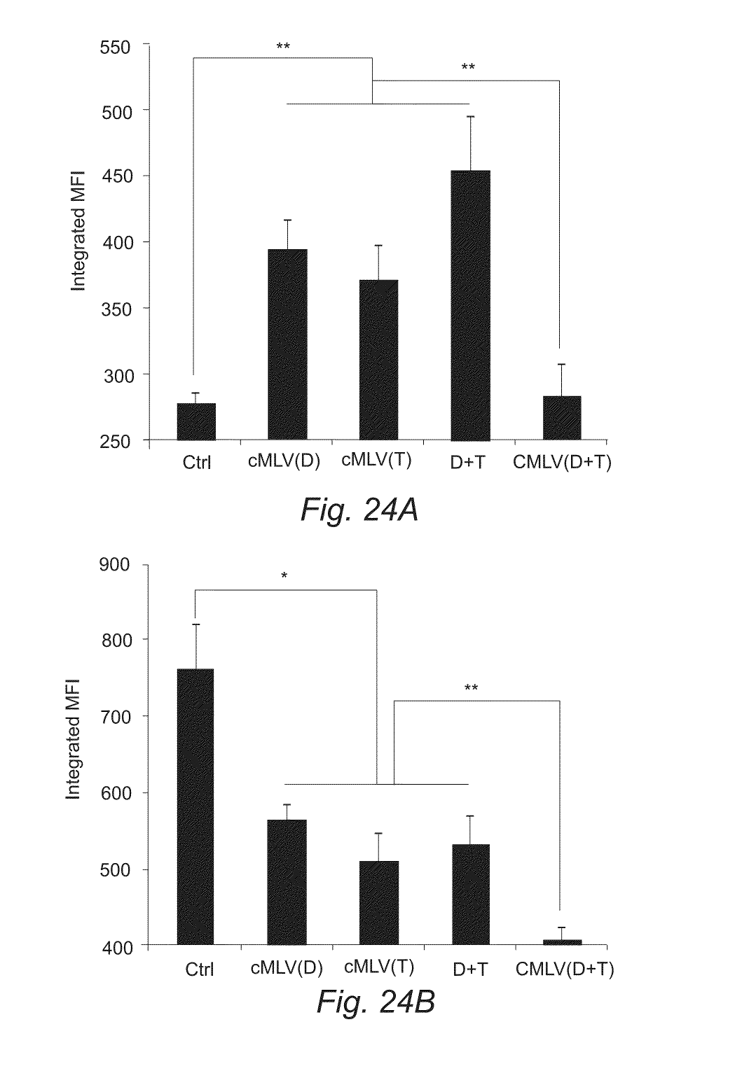

Having shown that dual drug-loaded cMLVs enhance cellular accumulation of drugs, we next sought to verify that this did, indeed, result from the modulation of membrane pumps, which are responsible for multidrug resistance. We first measured the expression of P-gp by flow cytometry in 4T1 cells treated with various nanoparticle formulations for 48 h to test if these cMLV formulations were responsible for altering P-gp involvement in multidrug resistance, along with decreased drug accumulation, in cells. As shown in FIG. 24A, with the single drug-loaded cMLV treatment, the expression of P-gp (in terms of integrated mean fluorescence intensity) increased significantly in 4T1 cells (p<0.01), possibly leading, in turn, to the development of drug resistance in 4T1 cells. However, dual drug-loaded cMLVs significantly inhibited expression of P-gp when compared to that of the single drug-loaded cMLVs and drug combination in solution (p<0.01), suggesting that the combinatorial delivery of Dox and PTX via cMLVs could efficiently suppress P-gp expression, thereby overcoming MDR. We next investigated whether cMLV(Dox+PTX) could inhibit multidrug resistance in JC cells, which exhibit drug-resistant phenotype by overexpression of P-gp. As shown in FIG. 24B, the expression of P-gp decreased after 48 h of incubation with JC cells (p<0.05) when treated with single drug-loaded cMLV, indicating that the nanoparticle drug delivery system could, at least partially, suppress MDR. However, the codelivery formulation of cMLV(Dox+PTX) significantly inhibited P-gp expression compared to that of single drug-loaded cMLVs and drug combination in solution (p<0.01). Taken together, these results indicated that the codelivery of Dox and PTX via cMLVs could inhibit the expression of P-gp and increase cellular accumulation of drugs, leading to enhanced drug action in cells, including drug-resistant cells.

Efficacy of Dual Drug-Loaded cMLVs Against a Murine Breast Cancer Model.

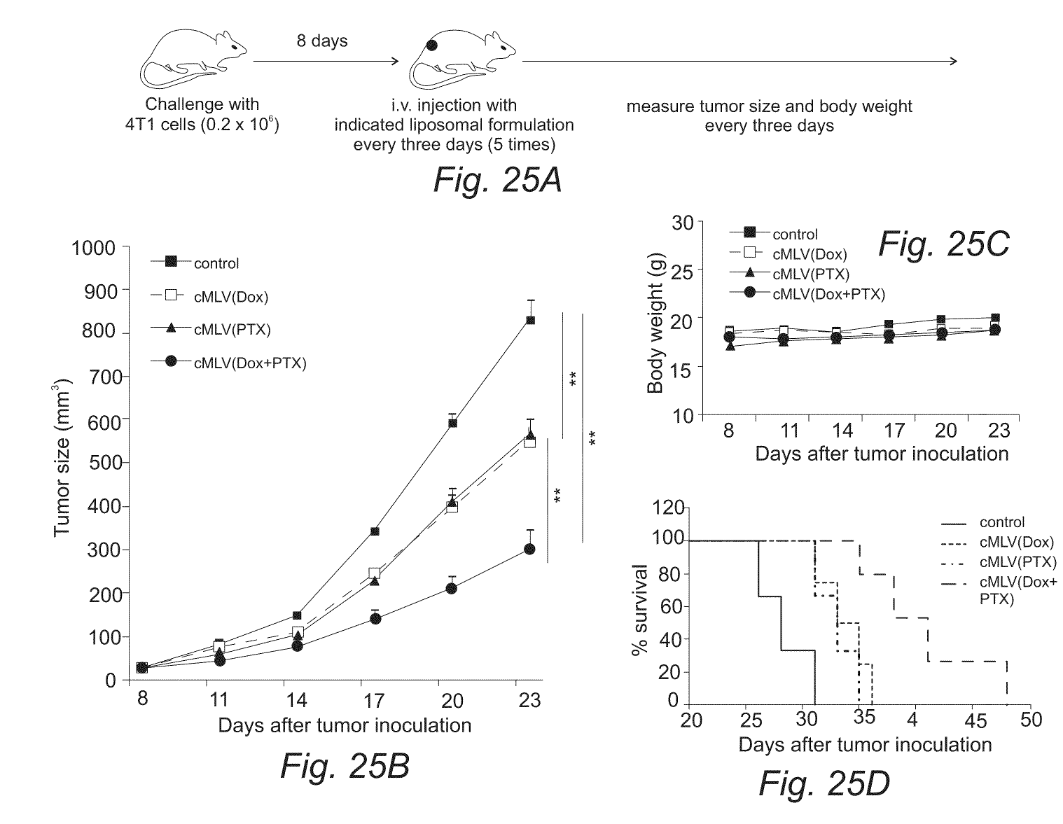

It has been demonstrated that codelivery of Dox and PTX via cMLVs is able to overcome drug resistance in vitro. However, since the in vivo environment is considerably more complicated, it remains unknown if this effect could be translated to an animal cancer model. Therefore, in this experiment, a mouse breast tumor model was used to evaluate the therapeutic efficacy of dual drug-loaded cMLVs compared with that of single-drug liposomal formulations. At day 0, BALB/c mice were inoculated subcutaneously with 4T1 breast tumor cells. On day 8, mice bearing tumors were randomly sorted into six groups, and each group was treated with one of the following: PBS (control), cMLV(2 mg/kg Dox), cMLV(2 mg/kg PTX), or cMLV(2 mg/kg Dox+2 mg/kg PTX) every three days. Tumor growth and body weights were monitored until the end of the experiment (FIG. 25A).

As shown in FIG. 25B, mice in groups receiving cMLV(Dox) or cMLV (PTX) exhibited tumor inhibition compared to those in the control group (p<0.01). Even more significantly, cMLV(Dox+PTX) treatment induced a greater inhibition than that of cMLV encapsulating a single drug (p<0.01). No weight loss was seen over the duration of the experiment (FIG. 25C), indicating the absence of any obvious systemic toxicity from this codelivery system. The in vivo efficacy of dual drug-loaded cMLVs against the 4T1 tumor model was further confirmed by a survival test. As shown in FIG. 25D, the groups treated with cMLV(Dox) or cMLV(PTX) had a prolonged lifespan compared to the control group, while the mice in the group treated with cMLV(Dox+PTX) had a significantly increased lifespan compared to the groups treated with single drug-loaded cMLVs (p<0.01).

Histology Study.

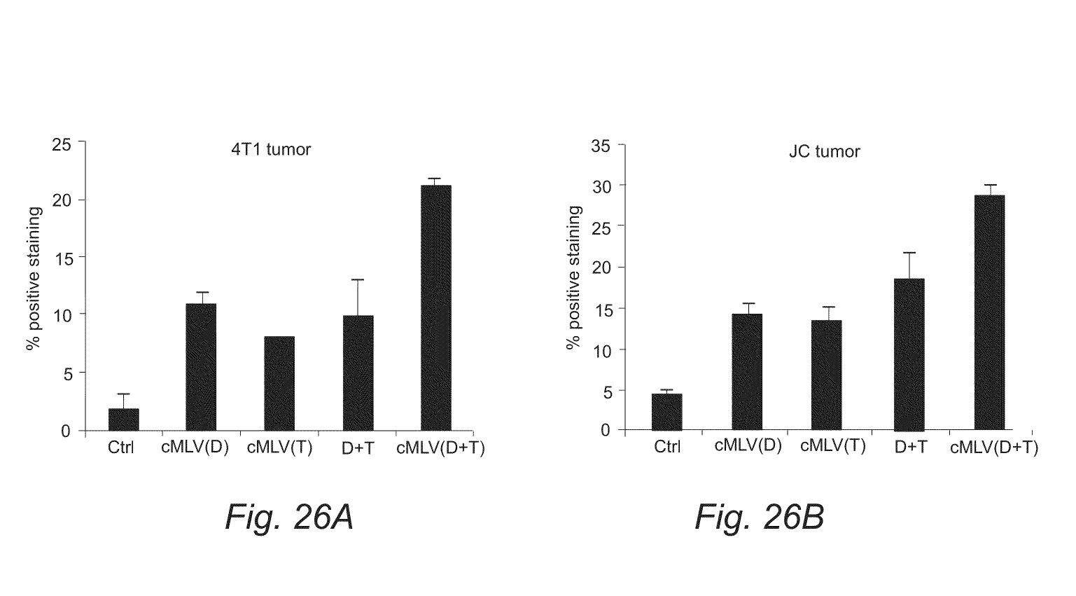

To study the antitumor mechanism in vivo, a TUNEL assay was carried out to detect tumor cell apoptosis in tumors treated with Dox (5 mg/kg) and/or PTX (5 mg/kg) in various formulations for 3 days. 4T1 tumors treated with cMLV(Dox), cMLV(PTX), and Dox+PTX in solution showed significantly more apoptotic cells compared with controls (p<0.01) (FIG. 26 A). The apoptosis index was also significantly higher in the cMLV(Dox+PTX)-treated group as compared with other groups (p<0.05). Thus, the efficacy of cMLV(Dox+PTX) as an antitumor treatment could be explained by data suggesting increased tumor cell apoptosis. To further confirm the induction of cell apoptosis in treated groups, the TUNEL assay was performed in drug-resistant JC tumors treated with various formulations for 3 days (FIG. 26 B). cMLV(Dox), cMLV(PTX), and Dox+PTX induced more apoptotic cells compared to control JC tumors (p<0.01). Dual drug-loaded cMLV-treated JC tumors showed a remarkably higher apoptosis index compared with other groups (p<0.01), again confirming the enhanced antitumor activity of cMLV(Dox+PTX).

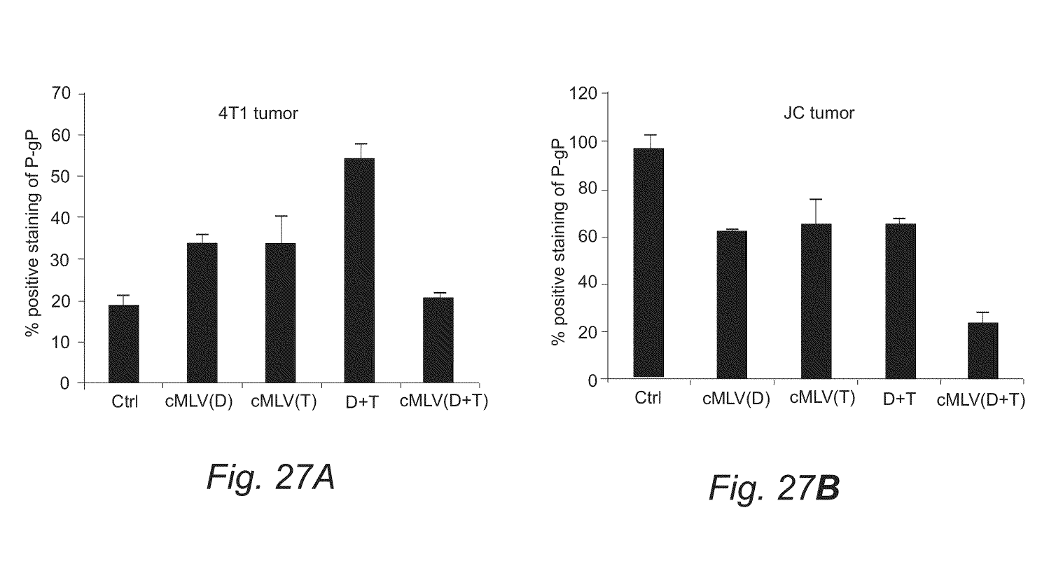

To further investigate the innate characteristics of treated tumors, both 4T1 and JC tumor sections from each treatment group were analyzed for the expression of P-gp protein. As shown in FIG. 27A, P-gp expression level was moderate in the control group. There appeared to be a significant enhancement of P-gp expression in the cMLV(Dox) and cMLV(PTX) groups, with an even more significant enhancement in Dox+PTX group compared to controls. However, a marked decrease was observed in the cMLV(Dox+PTX)-treated group when compared to the cMLV(Dox), cMLV(PTX), and Dox+PTX groups, as further confirmed by the quantification data in FIG. 27A (p<0.01). Interestingly, P-gp was very high in the JC tumor control group. However, a significant decrease appeared in the cMLV(Dox), cMLV(PTX), and Dox+PTX groups, as further confirmed by the quantification data in FIG. 27C (p<0.05). An even more significant decrease of P-gp expression was seen in the cMLV(Dox+PTX) group (p<0.01), indicating that dual drug-loaded cMLVs might be able to alter the innate characteristics of the multidrug-resistant tumor cells such as JC cells. Taken together, these data show that drug-loaded nanoparticles can partially bypass the P-gp efflux pumps to increase cellular uptake of Dox and PTX, sufficiently inducing cytotoxicity in cancer cells.

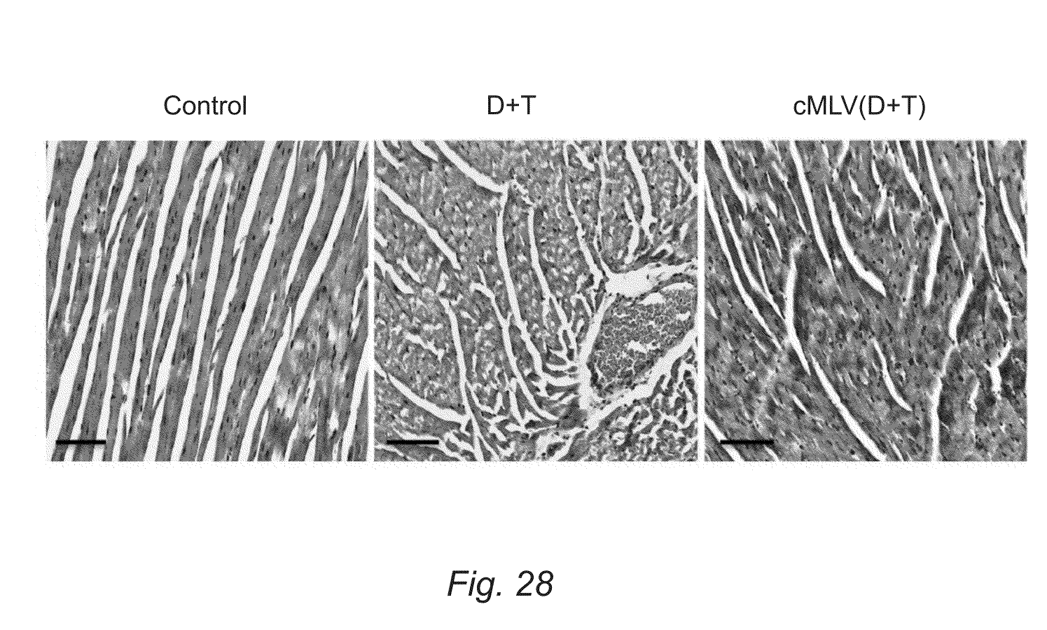

It has been reported that Dox treatment results in severe irreversible cardiotoxicity, leading to myocyte apoptosis. In addition, cardiac toxicity, an unexpected clinical outcome of combinatorial Dox and PTX treatment, has been reported. Therefore, systemic toxicity of free Dox+PTX and cMLV(Dox+PTX) was evaluated to determine whether codelived cMLVs could decrease this side effect of combination drug treatment. To accomplish this, a single intravenous dose of either Dox+PTX in solution or cMLV(Dox+PTX) was administered to mice bearing 4T1 tumors. Next, hematoxylin and eosin-stained cardiac tissue sections from each treatment group were examined (FIG. 28). Treatment with free Dox (5 mg/kg) and PTX (5 mg/kg) in solution did cause cardiac toxicity, as indicated by myofibril loss, disarray, and cytoplasmic vacuolization. However, when cMLV(5 mg/kg Dox+5 mg/kg PTX) was administered under the same experimental conditions via cMLVs, no visible loss of myocardial tissue was observed.

### Discussion

Chemotherapeutics are crucial to combating a variety of cancers; however, clinical outcomes are always poor, as cancer cells develop a multidrug resistance (MDR) phenotype after several rounds of exposure to the chemotherapeutics. Many efforts have been made to develop a therapeutic strategy to overcome tumor MDR through the use of combined therapeutics to enhance the efficiency of systemic drug delivered to the tumor site and lower the apoptotic threshold. In this study, we have examined augmentation of therapeutic efficacy upon co-administration of Dox and PTX using a crosslinked multilamellar liposomal vesicle (cMLV) in breast cancer cells and drug-resistant JC cells. We demonstrated that combination therapy of Dox and PTX, especially when codelivered in cMLV formulations, was effective in enhancing the cytotoxicity in both wild-type and drug-resistant cells by elevating the cellular accumulation and retention of the drugs. We also showed that the dual therapeutic strategy efficiently suppressed tumor growth by enhancing apoptotic response.

P-glycoprotein (P-gp), a membrane-bound active drug efflux pump, is considered one of the most important mechanisms involved in MDR. As a result, growing interest has been shown in the development of nanoparticle drug delivery systems to overcome MDR. With their unique properties, nanoparticles are able to passively target the tumor mass through the enhanced permeability and retention (EPR) effect, enhancing the accumulation of chemotherapeutics at target sites. In addition, nanoparticles can enter cells through the endocytosis pathway, which is thought to be independent of the P-gp pathway, thus increasing the cellular uptake and retention of therapeutics in resistant cancer cells. Previously, we demonstrated the advantage of cMLVs in cancer therapy over conventional liposomal formulations based on their sustained drug release, enhanced vesicle stability and improved drug release, resulting in improved therapeutic activity with reduced systemic toxicity. Moreover, cMLVs are internalized by tumor cells through clathrin-mediated endocytosis, suggesting that cMLVs could be an efficient drug carrier to overcome MDR. In this study, our in vitro and in vivo results demonstrated that the co-administration of Dox and PTX via cMLVs efficiently suppressed P-gp expression in both wild-type and drug-resistant cancer cells.

In addition to nanodelivery, another potential strategy to overcome MDR has resulted from combining multiple drugs. For example, the combination of Dox and PTX in a cocktail is a standard anthracycline-taxane treatment regimen and was found to be efficacious in treating a variety of tumors by reducing the individual drug concentration that would otherwise be required to achieve cytotoxicity, thus overcoming drug resistance However, its clinical outcome was limited by the un-coordinated biodistribution of combined drugs and increase in cardiac cytotoxicity. In this study, the pharmacokinetics of Dox and PTX was unified through the encapsulation of both drugs into a single cMLV particle, resulting in dual drug-loaded cMLVs which successfully reduced P-gp expression, increased the cellular accumulation of drugs, and enhanced cytotoxicity in cancer cells, including drug-resistant cells, as compared to single drug-loaded cMLVs. Moreover, combination therapy of Dox and PTX administered in cMLV formulations showed increased efficacy over cMLV monotherapy in the suppression of tumor growth by promoting apoptotic response in vivo.

5. Multilamellar Liposomes Encapsulating siRNAs

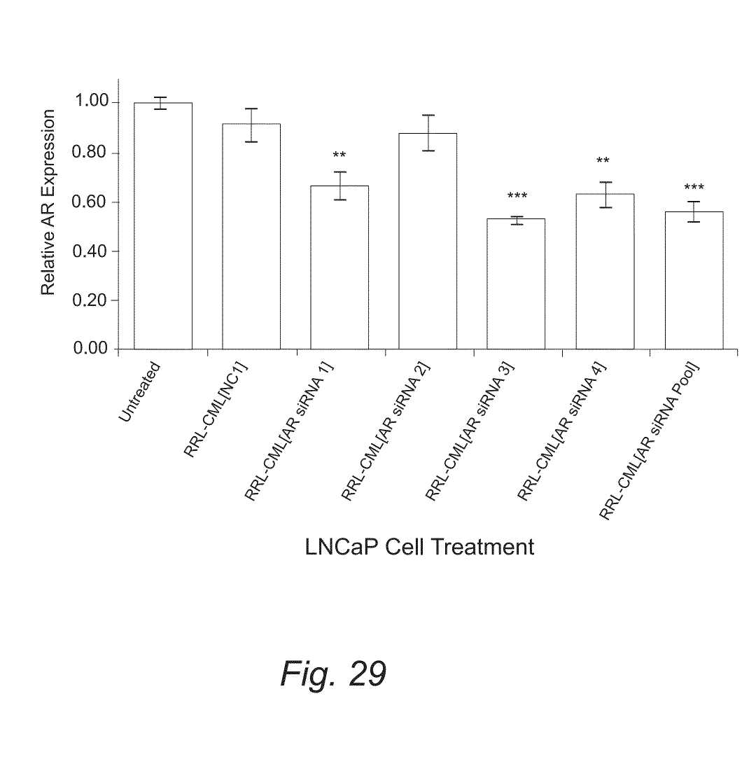

To initially visualize that RRL-CML nanoparticles are capable of meeting the siRNA encapsulation and delivery criteria, DiD-labelled siRNAs were encapsulated into CML nanoparticles and incubated with human cancer cells. Within one hour, encapsulated siRNAs had been delivered to the cytoplasm of the tumor cells. To demonstrate that multiple functional siRNAs can be delivered specifically to prostate cancer cells by RRL-CML nanoparticles, four different siRNAs targeting the AR were designed. These were encapsulated into RRL-CML nanoparticles both individually and together as a pool and then incubated with human LNCaP prostate cancer cells in standard tissue culture conditions. In the case of the NC1, the siRNA was encapsulated at 5 micromolar in 200 microliters total volume. In all other cases, siRNAs (i.e. 1-4 and the pool) were encapsulated at a total of 10 micromolar in 200 microliters total volumeTreated cells were harvested after 48 hours, and AR expression assessed by real-time quantitative PCR (FIG. 29). As negative controls, both untreated LNCaP cells and LNCaP cells incubated with RRL-CML nanoparticles encapsulating a universal negative control siRNA (NC1, Integrated DNA Technologies, Coralville, Iowa) were used. All but one of the individual anti-AR RRL-CML[siRNA] nanoparticles mediated strongly statistically significant androgen receptor knockdown, as did the nanoparticle encapsulating the anti-AR siRNA pool. No significant knockdown was mediated by the RRL-CML[NC1] negative control nanoparticle compared to untreated LNCaP cells. With optimization, we expect to be able to dramatically improve the knockdown efficiency mediated by RRL-CML[siRNA] nanoparticles demonstrated in this initial test. This data demonstrates unequivocally that RRL-CML nanoparticles can indeed successfully deliver multiple therapeutic siRNAs to human prostate cancer cells.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A composition comprising:
a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer; and
at least one anticancer compound within the liposome.

2. The composition of claim 1 wherein the multilamellar liposome includes at least one additional lipid bilayer covalently bonded to an adjacent lipid bilayer.

3. The composition of claim 1 wherein the at least one anticancer compound includes a hydrophobic anticancer compound disposed in lipid bilayers and a hydrophilic anticancer compound disposed within the central liposome cavity.

4. The composition of claim 1 wherein the first lipid bilayer and the second lipid bilayer are covalently bonded by thioether bonds.

5. The composition of claim 1 wherein the first lipid bilayer and the second lipid bilayer each independently include lipids with maleimide-headgroups.

6. The composition of claim 5 wherein the first lipid bilayer and the second lipid bilayer each independently include a maleimide-containing diacylglycerol lipid.

7. The composition of claim 5 wherein the first lipid bilayer and the second lipid bilayer each independently include a sodium salt of a compound elected from the group consisting of 1,2-dioleoyl-sn-glycero-3-phosphoethanolamine-N-[4-(p-maleimidomethyl)cyclohexane-carboxamide], 1,2-dioleoyl-sn-glycero-3-phosphoethanolamine-N-[4-(p-maleimidophenyl)butyramide], 1,2-dipalmitoyl-sn-glycero-3-phosphoethanolamine-N-[4-(p-maleimidomethyl)cyclohexane-carboxamide], and 1,2-dipalmitoyl-sn-glycero-3-phosphoethanolamine-N-[4-(p-maleimidophenyl)butyramide], and combinations thereof.

8. The composition of claim 5 wherein the first lipid bilayer and the second lipid bilayer each independently include phospholipids that are different than the lipids with maleimide-headgroups.

9. The composition of claim 5 wherein the first lipid bilayer and the second lipid bilayer each independently include a phospholipid that is a fatty acid di-ester of phosphatidylcholine, ethylphosphatidylcholine, phosphatidylglycerol, phosphatidic acid, phosphatidylethanolamine, phosphatidylserine or sphingomyelin.

10. The composition of claim 1 wherein the at least one anticancer compound includes a component selected from the group consisting of a DNA alkylating agent, oxidant, topoisomerase inhibitor, and combinations thereof.

11. The composition of claim 1 wherein the at least one anticancer compound includes a component selected from the group consisting of methyl methanesulfonate, cyclophosphamide, etoposide, doxorubicin, Taxol (paclitaxel), menadione, taxotere (docetaxel), rapamycin, carboplatinum, cisplatinum, gemcitabine, doxorubicin, siRNAs, and combinations thereof.

12. The composition of claim 1 wherein the at least one anticancer compound includes a component selected from the group consisting of doxorubicin and taxol.

13. The composition of claim 1 wherein the at least one anticancer compound includes at least one siRNA.

14. The composition of claim 13, wherein the at least one siRNA is directed to an androgen receptor.

15. The composition of claim 1 wherein a poly(ethylene glycol) group is covalently bonded to the exterior surface of the liposome.

16. A composition comprising:
a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer;
a targeting peptide covalently bonded to the exterior surface of the multilamellar liposome; and
at least one anticancer compound disposed within the liposome.

17. The composition of claim 16 wherein the multilamellar liposome includes at least one additional lipid bilayer.

18. The composition of claim 16 wherein the first lipid bilayer and the second lipid bilayer are covalently bonded by thioether bonds.

19. The composition of claim 16 wherein the first lipid bilayer and the second lipid bilayer each independently include lipids with maleimide-headgroups.

20. The composition of claim 19 wherein the first lipid bilayer and the second lipid bilayer are each independently include a maleimide-containing diacylglycerol lipid.

21. The composition of claim 16 wherein the targeting peptide is circular.

22. The composition of claim 16 wherein the targeting peptide includes a peptide sequence selected from the group consisting of RRL, RGD, CGGRRLGGC, and CRGDKGPDC.

23. The composition of claim 16 wherein the first lipid bilayer and the second lipid bilayer each independently include a phospholipid that is a fatty acid di-ester of phosphatidylcholine, ethylphosphatidylcholine, phosphatidylglycerol, phosphatidic acid, phosphatidylethanolamine, phosphatidylserine or sphingomyelin.

24. The composition of claim 16 wherein the at least one anticancer compound includes a component selected from the group consisting of a DNA alkylating agent, oxidant, topoisomerase inhibitor, and combinations thereof.

25. The composition of claim 16 wherein the at least one anticancer compound includes a component selected from the group consisting of methyl methanesulfonate, cyclophosphamide, etoposide, doxorubicin, Taxol (paclitaxel), menadione, taxotere (docetaxel), rapamycin, carboplatinum, cisplatinum, gemcitabine, doxorubicin, siRNAs, and combinations thereof.

26. The composition of claim 16 wherein the at least one anticancer compound includes a component selected from the group consisting of doxorubicin and taxol.

27. A method for treating cancer, the method comprising:
identifying a subject having cancer; and
administering a therapeutically effective amount of a composition comprising:
a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer; and
at least one anticancer compound disposed within the liposome.

28. The method of claim 27 further comprising a targeting peptide covalently bonded to the exterior surface of the multilamellar liposome.

29. The method of claim 28 wherein the targeting peptide is within a ring structure.

30. The method of claim 29 wherein the targeting peptide includes a peptide sequence selected from the group consisting of RRL, RGD, CGGRRLGGC, and CRGDKGPDC.

31. The method of claim 29 wherein the targeting peptide includes a peptide sequence having formula 1: 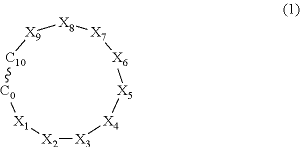
wherein C₀ and C₁₀ are each independently cysteine, a wavy line is a disulfide bond, and X₁-X₉ are absent or an amino acid residues with the proviso that at least one sequence in X₁-X₉ is RRL, RGD, GGRRLGG, or RGDKGPD.

32. The method of claim 29 wherein the targeting peptide includes a peptide sequence having formula 2: 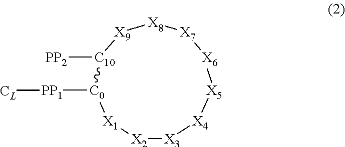
wherein C_{L} is a cysteine that bonds to the liposomes set forth above via a thioether bond, C₀ and C₁₀ are each independently cysteine, PP₁ and PP₂ are each independently absent or an arbitrary polypeptide having from 1 to 10 amino acid residues; and X₁-X₉ are each independently absent or an amino acid residues with the proviso that at least one sequence in X₁-X₉ is RRL, RGD, GGRRLGG, or RGDKGPD.

33. The method of claim 27 wherein the at least one anticancer compound includes a component selected from the group consisting of methyl methanesulfonate, cyclophosphamide, etoposide, doxorubicin, Taxol (paclitaxel), menadione, taxotere (docetaxel), rapamycin, carboplatinum, cisplatinum, gemcitabine, doxorubicin, siRNAs, and combinations thereof.

34. The method of claim 27 wherein the at least one anticancer compound includes a hydrophilic compound and a hydrophobic compound.

35. The method of claim 27 wherein the weight ratio of the hydrophilic compound to the hydrophobic compound is from about 1:5 to 5:1.

36. The method of claim 27 wherein the weight ratio of the hydrophilic compound to the hydrophobic compound is from about 3:3 to 5:1.

37. The method of claim 27 wherein the hydrophilic compound is doxorubicin and the hydrophobic compound is paclitaxel.

38. A method for treating cancer, the method comprising:
identifying a subject having multidrug resistant cancer; and
administering a therapeutically effective amount of a composition comprising:
a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer; and
at least one anticancer compound disposed within the liposome.

39. The method of claim 38 wherein multidrug resistant cancer is identified by determining the expression of P-glycoprotein (P-gp) in cancer cells.

40. The method of claim 38 further comprising a targeting peptide covalently bonded to the exterior surface of the multilamellar liposome.

41. The method of claim 40 wherein the targeting peptide is within a ring structure.

42. The method of claim 41 wherein the targeting peptide includes a peptide sequence selected from the group consisting of RRL, RGD, CGGRRLGGC, and CRGDKGPDC.

43. The method of claim 38 wherein the at least one anticancer compound includes a component selected from the group consisting of methyl methanesulfonate, cyclophosphamide, etoposide, doxorubicin, Taxol (paclitaxel), menadione, taxotere (docetaxel), rapamycin, carboplatinum, cisplatinum, gemcitabine, doxorubicin, siRNAs, and combinations thereof.

44. The method of claim 38 wherein the at least one anticancer compound includes a hydrophilic compound and a hydrophobic compound.

45. The method of claim 38 wherein the weight ratio of the hydrophilic compound to the hydrophobic compound is from about 1:5 to 5:1.

46. The method of claim 38 wherein the weight ratio of the hydrophilic compound to the hydrophobic compound is from about 3:3 to 5:1.

47. The method of claim 46 wherein the hydrophilic compound is doxorubicin and the hydrophobic compound is paclitaxel.


SEQUENCE LISTING (13 sequences)

SEQ ID NO: 1 - protein, 9 aa, Artificial Sequence
  General synthetic targeting peptide
XXXXXXXXX

SEQ ID NO: 2 - protein, 11 aa, Artificial Sequence
  General synthetid targeting peptide
CXXXXXXXXXC

SEQ ID NO: 3 - DNA, 21 bp, Artificial Sequence
  synthetic siRNA forward sequence
cuacaacuuuccacuggcutt

SEQ ID NO: 4 - DNA, 21 bp, Artificial Sequence
  synthetic siRNA reverse sequence to sequnece 3
agccaguggaaaguuguagtt

SEQ ID NO: 5 - DNA, 21 bp, Artificial Sequence
  synthetic siRNA forward sequence
gcugcaaggucuucuucaatt

SEQ ID NO: 6 - DNA, 21 bp, Artificial Sequence
  synthetic siRNA reverse sequence to sequence 5
uugaagaagaccuugcagctt

SEQ ID NO: 7 - DNA, 21 bp, Artificial Sequence
  synthetic siRNA forward sequence
gcagaaaugauugcacuautt

SEQ ID NO: 8 - DNA, 21 bp, Artificial Sequence
  synthetic siRNA reverse sequence to sequence 7
gcagaaaugauugcacuautt

SEQ ID NO: 9 - DNA, 21 bp, Artificial Sequence
  synthetic siRNA forward sequence
auuaccaaguuucuucagctt

SEQ ID NO: 10 - DNA, 21 bp, Artificial Sequence
  synthetic siRNA reverse sequence to sequence 10
auuaccaaguuucuucagctt

SEQ ID NO: 11 - DNA, 144 bp, Homo sapiens
gaccatgttttgcccattgactattactttccaccccagaagacctgcctgatctgtggagatgaagcttctgggtgtcactatggagctctcacatgtggaagctgcaaggtcttcttcaaaagagccgctgaagggaaacag

SEQ ID NO: 12 - DNA, 123 bp, Homo sapiens
aagtacctgtgcgccagcagaaatgattgcactattgataaattccgaaggaaaaattgtccatcttgtcgtcttcggaaatgttatgaagcagggatgactctgggagcccggaagctgaag

SEQ ID NO: 13 - DNA, 291 bp, Homo sapiens
aaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacac